(19) United States
(12) Patent Application Publication
(10) Pub. No.: US 2010/0113513 A1
(43) Pub. Date: May 6, 2010

(54) QUINOLINE DERIVATIVES AS FUNGICIDES

(75) Inventors: Fiona Murphy Kessabi, Stein (CH); Renaud Beaudegnies, Stein (CH); Laura Quaranta, Stein (CH); Hans-Georg Brunner, Stein (CH); Fredrik Cederbaum, Stein (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/531,198

(22) PCT Filed: Mar. 12, 2008
(86) PCT No.: PCT/EP08/01974

§ 371 Date: Sep. 14, 2009

(30) Foreign Application Priority Data
Mar. 14, 2007 (GB) 0704906.7

(51) Int. Cl. A01N 43/42; C07D 215/20; A01P 3/00

(52) U.S. Cl. 514/311; 546/175

(57) ABSTRACT

Compounds of the general formula (I) wherein the substituents are as defined in claim 1, are useful as fungicides.

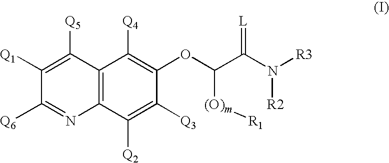

This invention relates to novel quinolinyloxyalkanoic acid amides, processes for preparing them, to compositions containing them and to methods of using them to combat fungi, especially fungal infections of plants.

Certain quinolinyloxyalkanoic acid amide derivatives and their use as agricultural and horticultural bactericides are disclosed, for example, in WO 04/047538 and JP 2001-89453.

The present invention is concerned with the provision of particular substituted quinoline-6-yloxyalkanoic acid amides for use mainly as plant fungicides.

Thus according to the present invention there is provided a compound of the general formula I

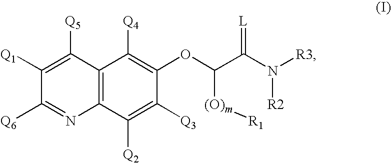

wherein
Q¹, Q², Q³, Q⁴, Q⁵ and Q⁶ independently of each other, are hydrogen, halogen, cyano, nitro, azido, optionally substituted C₁₋₆ alkyl, optionally substituted C₃₋₆ cycloalkyl, optionally substituted C₃₋₆ cycloalkyl(C₁₋₄)alkyl, optionally substituted C₂₋₆ alkenyl, optionally substituted C₂₋₆ alkynyl, optionally substituted C₁₋₆ alkoxy, optionally substituted C₂₋₆ alkenyloxy, optionally substituted C₂₋₆ alkynyloxy, optionally substituted aryl, optionally substituted aryloxy, optionally substituted aryl(C₁₋₆)alkyl, optionally substituted aryl(C₁₋₆)alkoxy, optionally substituted heteroaryl, optionally substituted heteroaryloxy, optionally substituted heteroaryl(C₁₋₆)alkyl, optionally substituted heteroaryl(C₁₋₆)alkoxy, —SF₅ or —S(O)ᵤ(C₁₋₆)alkyl, wherein u is 0, 1 or 2 and the alkyl group is optionally substituted with halogen, or
Q¹, Q², Q³, Q⁴, Q⁵ and Q⁶, independently of each other, are —OSO₂(C₁₋₄)alkyl, wherein the alkyl group is optionally substituted with halogen, or
Q¹, Q², Q³, Q⁴, Q⁵ and Q⁶, independently of each other, are —CONR^{u}R^{v}, —COR^{u}, —CO₂R^{u}, —CR^{u}═NR^{v}, —NR^{u}R^{v}, —NR^{u}COR^{v}, —NR^{u}CO₂R^{v}, —SO₂NR^{u}R^{v} or —NR^{u}SO₂R^{w}, wherein R^{w} is optionally substituted C₁₋₆ alkyl and R^{u} and R^{v} independently of each other, are hydrogen or C₁₋₆ alkyl optionally substituted with halogen, or, in the case of —CONR^{u}R^{v} or —SO₂NR^{u}R^{v}, R^{u}R^{v} may join to form a 5- or 6-membered carbocyclic or heterocyclic ring containing a heteroatom selected from sulfur, oxygen and NR^{o}, wherein R^{o} is hydrogen or optionally substituted C₁₋₆alkyl, or, in the case of —CR^{u}═NR^{v}, R^{v} is hydrogen, hydroxyl, or C₁₋₆alkoxy,
R¹ is C₁₋₄ alkyl, C₃₋₅ cycloalkyl, C₂₋₄ alkenyl or C₂₋₄ alkynyl in which the alkyl, alkenyl and alkynyl groups are optionally substituted on their terminal carbon atom with one, two or three halogen atoms, with a cyano group, with a C₁₋₄ alkylcarbonyl group, with a C₁₋₄ alkoxycarbonyl group or with a hydroxy group, or R₁ is alkoxyalkyl, alkylthioalkyl, alkylsulphinylalkyl or alkylsulphonylalkyl in which the total number of carbon atoms is 2 or 3, or R₁ is a straight-chain C₁₋₄ alkoxy group;
R² is hydrogen, C₁₋₈ alkyl, C₃₋₄ cycloalkyl, C₂₋₈ alkenyl, cyano(C₁₋₄)alkyl, C₁₋₄ alkoxy(C₁₋₄)-alkyl, C₁₋₄ alkoxy(C₁₋₄)alkoxy(C₁₋₄)alkyl or benzyloxy(C₁₋₄)alkyl, wherein the phenyl ring is optionally substituted with C₁₋₄ alkoxy,
R³ is —(CR^{a}R^{b})ₚ(CR^{c}R^{d})_{q}(X)ᵣ(CR^{e}R^{f})ₛR⁴, wherein
R^{a}, R^{b}, R^{c}, R^{d}, R^{e} and R^{f}, independently of each other, are hydrogen, C₁₋₄ alkyl, halogen, cyano, hydroxy, C₁₋₄ alkoxy or C₁₋₄ alkoxycarbonyl, or
R^{a}R^{b}, R^{c}R^{d} or R^{e}R^{f} may join to form a 3 to 8 membered carbocyclic or heterocyclic ring containing a heteroatom selected from sulfur, oxygen and NR^{o}, wherein R^{o} is hydrogen or optionally substituted C₁₋₆alkyl,
X is (CO), (CO)O, O(CO), O, S(O)ₜ, wherein t is 0, 1 or 2, or X is NH or N(C₁₋₆)alkyl,
p, r and s, independently of each other, are 0 or 1,
q is 0, 1 or 2,
R⁴ is optionally substituted C₁₋₆ alkyl, optionally substituted C₂₋₆ alkenyl or when at least one of p, q, r and s is 1, R⁴ is —CH₂—C≡C—R⁵, wherein
R⁵ is hydrogen, C₁₋₈ alkyl optionally substituted with halogen, hydroxy, C₁₋₆ alkoxy, C₁₋₃ alkoxy(C₁₋₃)alkoxy, cyano, C₁₋₄ alkylcarbonyloxy, aminocarbonyloxy, mono- or di(C₁₋₄)-alkylaminocarbonyloxy, tri(C₁₋₄)alkylsilyloxy or —S(O)_{g}(C₁₋₆)alkyl, wherein g is 0, 1 or 2, or
R⁵ is C₃₋₆ cycloalkyl optionally substituted with halogen, hydroxy, C₁₋₆ alkoxy, C₁₋₃ alkoxy-(C₁₋₃)alkoxy, cyano, C₁₋₄ alkylcarbonyloxy, aminocarbonyloxy, mono- or di(C₁₋₄)alkyl-aminocarbonyloxy, tri(C₁₋₄)alkylsilyloxy or —S(O)_{g}(C₁₋₆)alkyl, wherein g is 0, 1 or 2, or
R⁵ is C₃₋₆ cycloalkyl(C₁₋₄)alkyl, wherein the alkyl and/or cycloalkyl moiety is optionally substituted with halogen, hydroxy, C₁₋₆ alkoxy, C₁₋₃ alkoxy(C₁₋₃)alkoxy, cyano, C₁₋₄ alkylcarbonyloxy, aminocarbonyloxy, mono- or di(C₁₋₄)alkylaminocarbonyloxy, tri(C₁₋₄)alkylsilyloxy or —S(O)_{g}(C₁₋₆)alkyl, wherein g is 0, 1 or 2, or
R⁵ is optionally substituted aryl, optionally substituted aryl(C₁₋₄)alkyl, optionally substituted aryloxy(C₁₋₄)alkyl, optionally substituted heteroaryl or optionally substituted heteroaryl(C₁₋₄)alkyl or optionally substituted heteroaryloxy(C₁₋₄)alkyl, or
R⁴ is optionally substituted C₃₋₆ cycloalkyl, optionally substituted C₅₋₆ cycloalkenyl, optionally substituted aryl, optionally substituted heteroaryl or an optionally substituted 5- to 8-membered ring optionally containing a heteroatom selected from sulfur, oxygen or NR⁰, wherein R^{o} is hydrogen or optionally substituted C₁₋₆alkyl, or
R² and R³ may join to form a 5- or 6-membered ring optionally substituted with halogen, C₁₋₄ alkyl, mono- or di-(C₁₋₄)alkylaminocarbonyl, and optionally containing a heteroatom selected from sulphur, oxygen and NR⁰⁰, wherein R⁰⁰ is C₁₋₄ alkyl optionally substituted with halogen, C₁₋₆ alkoxy or cyano, or R⁰⁰ is phenyl optionally substituted with nitro, C₁₋₄ alkyl, halo(C₁₋₄)alkyl, C₁₋₄ alkylcarbonyl or heteroaryl, or R² and R³ may join to form an optionally substituted 6,6-membered bicycle,
L is sulfur or oxygen, and
m is 0 or 1; and
salts and N-oxides of the compounds of the formula I.

The compounds of the invention contain at least one asymmetric carbon atom and may exist as enantiomers (or as pairs of diastereoisomers) or as mixtures of such. Further, when n is 1, the compounds of the invention are sulphoxides, which can exists in two enantiomeric forms, and the adjacent carbon can also exists in two enantiomeric forms. Compounds of general formula (I) can therefore exist as racemates, diastereoisomers, or single enantiomers, and the invention includes all possible isomers or isomer mixtures in all proportions. It is to be expected that for any given compound, one isomer may be more fungicidally active than another.

N-oxides of the compounds of the formula I preferably denote the N-oxides formed by the quinoline moiety.

The salts which the compounds of the formula I can form are preferably those formed by interaction of these compounds with acids. The term “acid” comprises mineral acids such as hydrogen halides, sulphuric acid, phosphoric acid etc. as well as organic acids, preferably the commonly used alkanoic acids, for example formic acid, acetic acid and propionic acid.

Except where otherwise stated, alkyl groups and alkyl moieties of alkoxy, alkylthio, etc., suitably contain from 1 to 6, typically from 1 to 4, carbon atoms in the form of straight or branched chains. Examples are methyl, ethyl, n- and iso-propyl and n-, sec-, iso- and tert-butyl. Where alkyl moieties contain 5 or 6 carbon atoms, examples are n-pentyl and n-hexyl. Examples of suitable optional substituents of alkyl groups and moieties include halo, hydroxy, C₁₋₄ alkoxy and C₁₋₄ alkoxy(C₁₋₄)alkoxy, cyano, optionally substituted aryl and optionally substituted heteroaryl. Where the optional substituent is halo, the haloalkyl group or moiety is typically monochloromethyl, monofluoromethyl, dichloromethyl, difluoromethyl, trichloromethyl or trifluoromethyl.

Except where otherwise stated, alkenyl and alkynyl moieties also suitably contain from 2 to 6, typically from 2 to 4, carbon atoms in the form of straight or branched chains. Examples are allyl, ethynyl and propargyl. Optional substituents include halo, alkoxy, optionally substituted aryl and optionally substituted heteroaryl.

Halo includes fluoro, chloro, bromo and iodo.

Aryl is usually phenyl but also includes naphthyl, anthryl and phenanthryl.

Heteroaryl is typically a 5- or 6-membered aromatic ring containing one or more sulphur, oxygen or NR moieties as heteroatoms, which may be fused to one or more other aromatic or heteroaromatic rings, such as a benzene ring. Examples are thienyl, furyl, pyrrolyl, isoxazolyl, oxazolyl, thiazolyl, oxadiazolyl, pyrazolyl, imidazolyl, triazolyl, isothiazolyl, tetrazolyl, thiadiazolyl, pyridyl, pyrimidinyl, pyrazinyl, pyridazinyl, triazinyl, benzofuranyl, benzothienyl, dibenzofuranyl, dibenzothienyl, benzothiazolyl, benzoxazolyl, benzimidazolyl, indolyl, quinolyl, isoquinolyl, quinazolinyl and quinoxalinyl groups and, where appropriate, N-oxides and salts thereof. Any of the aryl or heteroaryl values are optionally substituted. Except where otherwise stated, substituents which may be present include one or more of the following: halo, hydroxy, mercapto, C₁₋₆ alkyl (especially methyl and ethyl), C₂₋₆ alkenyl (especially allyl), C₂₋₆ alkynyl (especially propargyl), C₁₋₆ alkoxy (especially methoxy), C₂₋₆ alkenyloxy (especially allyloxy), C₂₋₆ alkynyloxy (especially propargyloxy), halo(C₁₋₆)alkyl (especially trifluoromethyl), halo(C₁₋₆)alkoxy (especially trifluoromethoxy), —S(O)ₘ(C₁₋₆)alkyl wherein m is 0, 1 or 2 and the alkyl is optionally substituted with halo, hydroxy(C₁₋₆)alkyl, C₁₋₄ alkoxy(C₁₋₄)alkyl, C₁₋₄alkoxy(C₁₋₄)alkoxy, C₃₋₆ cycloalkyl, C₃₋₆ cycloalkyl(C₁₋₄)alkyl, optionally substituted aryl (especially optionally substituted phenyl), optionally substituted heteroaryl (especially optionally substituted pyridyl or pyrimidinyl), optionally substituted aryloxy (especially optionally substituted phenoxy), optionally substituted heteroaryloxy (especially optionally substituted pyridyloxy or pyrimidinyloxy), optionally substituted —S(O)ₘaryl wherein m is 0, 1 or 2 (especially optionally substituted phenylthio), optionally substituted —S(O)ₘheteroaryl wherein m is 0, 1 or 2 (especially optionally substituted pyridylthio or pyrimidinylthio), optionally substituted aryl(C₁₋₄)alkyl (especially optionally substituted benzyl, optionally substituted phenethyl and optionally substituted phenyl n-propyl) in which the alkyl moiety is optionally substituted with hydroxy, optionally substituted heteroaryl(C₁₋₄)alkyl (especially optionally substituted pyridyl- or pyrimidinyl-(C₁₋₄)alkyl), optionally substituted aryl(C₂₋₄)alkenyl (especially optionally substituted to phenylethenyl), optionally substituted heteroaryl(C₂₋₄)alkenyl (especially optionally substituted pyridylethenyl or pyrimidinylethenyl), optionally substituted aryl(C₁₋₄)alkoxy (especially optionally substituted benzyloxy and phenethyloxy), optionally substituted heteroaryl(C₁₋₄)alkoxy (especially optionally substituted pyridyl(C₁₋₄)alkoxy or pyrimidinyl(C₁₋₄)alkoxy), optionally substituted aryloxy(C₁₋₄)alkyl (especially phenoxymethyl), optionally substituted heteroaryloxy-(C₁₋₄)alkyl (especially optionally substituted pyridyloxy or pyrimidinyloxy(C₁₋₄)alkyl), optionally substituted —S(O)ₘ(C₁₋₄)alkylaryl wherein m is 0, 1 or 2 (especially optionally substituted benzylthio and phenethylthio), optionally substituted —S(O)ₘ(C₁₋₄)alkylheteroaryl wherein m is 0, 1 or 2 (especially optionally substituted pyridyl(C₁₋₄)alkylthio or pyrimidinyl(C₁₋₄)alkylthio), optionally substituted —(C₁₋₄)alkylS(O)ₘaryl wherein m is 0, 1 or 2 (especially phenylthiomethyl), optionally substituted —(C₁₋₄)alkyl S(O)ₘheteroaryl wherein m is 0, 1 or 2 (especially optionally substituted pyridylthio(C₁₋₄)alkyl or pyrimidinylthio(C₁₋₄)alkyl), acyloxy, including C₁₋₄ alkanoyloxy (especially acetyloxy) and benzoyloxy, cyano, isocyano, thiocyanato, isothiocyanato, nitro, NR^{g}R^{h}, —NHCOR^{g}, —NHCONR^{g}R^{h}, —CONR^{g}R^{h}, —CO₂R^{g}, —SO₂Rⁱ, —OSO₂Rⁱ, —COR^{g}, —CR^{g}═NR^{h} or —N═CR^{g}R^{h} in which Rⁱ is C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄ alkyl or C₁₋₄ alkoxy and R^{g} and R^{h} are independently hydrogen, C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄ alkyl or C₁₋₄ alkoxy.

Of particular interest are those compounds of the formula (I), wherein Q² is hydrogen, C₁₋₄ alkyl or halogen, Q¹, Q³, Q⁴, Q⁵ and Q⁶ are as defined above. Preferably, Q² is methyl or ethyl.

Of particular interest are those compounds of the formula (I), wherein Q¹ is halogen, aryl or heteroaryl, Q² is hydrogen, C₁₋₄ alkyl or halogen and Q³, Q⁴, Q⁵ and Q⁶ are as defined above. Preferably, Q² is methyl or ethyl.

Of particular interest are those compounds of the formula (I), wherein Q¹ is aryl, Q² is hydrogen, C₁₋₄ alkyl or halogen and Q³, Q⁴, Q⁵ and Q⁶ is as defined above. Preferably, Q² is methyl or ethyl.

Of particular interest are those compounds of the formula (I), wherein Q¹ is heteroaryl, Q² is hydrogen, C₁₋₄ alkyl or halogen and Q³, Q⁴, Q⁵ and Q⁶ is as defined above. Preferably, Q² is methyl or ethyl.

Of particular interest are those compounds of the formula (I), wherein Q¹ and Q³, independently of each other, are hydrogen or halogen and Q², Q⁴, Q⁵ and Q⁶ are hydrogen. Preferably, Q¹ and Q³, independently of each other, are fluoro, chloro, bromo, or iodo. More preferably, Q¹ is chloro, bromo or iodo and Q³ is fluoro or chloro.

Of particular interest are those compounds of the formula (I), wherein Q¹ is aryl or heteroaryl, Q², Q⁴, Q⁵ and Q⁶ are hydrogen and Q³ is hydrogen or halogen. Preferably, Q¹ is thiophen-2-yl, thiophen-3-yl, halo, or halo or alkoxy substituted phenyl or halo or alkoxy substituted pyridyl. It is also preferred that Q³ is hydrogen, fluoro or chloro.

Of particular interest are those compounds of the formula (I), wherein Q¹, Q², Q⁴, Q⁵ and Q⁶ are hydrogen and Q³ is hydrogen, halogen or optionally substituted alkyl. Preferably, Q³ is hydrogen, fluoro or chloro.

Of particular interest are those compounds of the formula (I), wherein Q¹ is halogen, Q², Q⁴, Q⁵ and Q⁶ are hydrogen and Q³ is hydrogen or optionally substituted alkyl. Preferably, Q¹ is chloro, bromo or iodo. It is also preferred that Q³ is methyl.

Of particular interest are those compounds of the formula (I), wherein Q¹ and Q² are halogen and Q³ is hydrogen or optionally substituted alkyl and Q⁴, Q⁵ and Q⁶ are hydrogen. Preferably, Q¹ is chloro, bromo or iodo. It is also preferred that Q³ is methyl.

Of particular interest are those compounds of the formula (I), wherein Q¹ is bromo and Q², Q³, Q⁴, Q⁵ and Q⁶, independently of each other, are hydrogen, C₁₋₄ alkyl or halogen. Preferably, Q² is halogen and Q³, Q⁴, Q⁵ and Q⁶ are hydrogen. It is also preferred that Q² is methyl or ethyl and Q³, Q⁴, Q⁵ and Q⁶ are hydrogen. Further, it is preferred that Q³ is fluoro or chloro and Q², Q⁴, Q⁵ and Q⁶ are hydrogen.

Of particular interest are those compounds of the formula (I), wherein Q¹ is iodo and Q², Q³, Q⁴, Q⁵ and Q⁶, independently of each other, are hydrogen, C₁₋₄ alkyl or halogen. Preferably, Q² is halogen and Q³, Q⁴, Q⁵ and Q⁶ are hydrogen. It is also preferred that Q² is methyl or ethyl and Q³, Q⁴, Q⁵ and Q⁶ are hydrogen. Further, it is preferred that Q³ is fluoro or chloro and Q², Q⁴, Q⁵ and Q⁶ are hydrogen.

Of particular interest are those compounds of the formula (I), wherein Q¹ is chloro and Q², Q³, Q⁴, Q⁵ and Q⁶, independently of each other, are hydrogen, C₁₋₄ alkyl or halogen. Preferably, Q² is halogen and Q³, Q⁴, Q⁵ and Q⁶ are hydrogen. It is also preferred that Q² is methyl or ethyl and Q³, Q⁴, Q⁵ and Q⁶ are hydrogen. Further, it is preferred that Q³ is fluoro or chloro and Q², Q⁴, Q⁵ and Q⁶ are hydrogen.

Of particular interest are those compounds of the formula (I), wherein Q¹ is chloro, bromo or iodo.

Of particular interest are those compounds of the formula (I), wherein Q¹ is fluoro.

Of particular interest are those compounds of the formula (I), wherein Q³ is hydrogen or halogen. Preferably, Q³ is hydrogen, fluoro or chloro. More preferably, Q³ is fluoro.

Of particular interest are those compounds of the formula (I), wherein Q¹ is bromo, Q², Q⁴, Q⁵ and Q⁶ are hydrogen and Q³ is hydrogen, fluoro or chloro. Preferably, Q³ is fluoro. It is also preferred that Q³ is chloro. Further, it is preferred that Q³ is hydrogen.

Of particular interest are those compounds of the formula (I), wherein Q¹ is iodo, Q², Q⁴, Q⁵ and Q⁶ are hydrogen and Q³ is hydrogen, fluoro or chloro. Preferably, Q¹ is iodo, Q², Q⁴, Q⁵ and Q⁶ are hydrogen and Q³ is fluoro. It is also preferred that Q¹ is iodo, Q², Q⁴, Q⁵ and Q⁶ are hydrogen and Q³ is chloro. Further, it is preferred that Q³ is hydrogen.

Of particular interest are those compounds of the formula (I), wherein Q¹ is hydrogen, halogen, optionally substituted C₂₋₆ alkenyl, optionally substituted C₂₋₆ alkynyl, optionally substituted aryl or optionally substituted heteroaryl.

Of particular interest are those compounds of the formula (I), wherein R¹ is C₁₋₄ alkyl. Preferably, R¹ is methyl or ethyl. More preferably, R¹ is methyl. It is also preferred that R¹ is ethyl.

Of particular interest are those compounds of the formula (I), wherein R¹ is methyl or ethyl, Q¹ is hydrogen or halogen, Q² is hydrogen, C₁₋₄ alkyl or halogen and Q³ is hydrogen or halogen. Preferably, Q¹ is chloro, bromo or iodo, Q² is hydrogen, methyl, ethyl, chloro or bromo, and Q³ is fluoro or bromo.

Of particular interest are those compounds of the formula (I), wherein R² is hydrogen or methyl. Preferably, R² is hydrogen.

Of particular interest are those compounds of the formula (I), wherein R¹ is methyl or ethyl, R² is hydrogen, Q¹ is hydrogen or halogen, Q² is hydrogen, C₁₋₄ alkyl or halogen and Q³ is hydrogen or halogen. Preferably, Q¹ is chloro, bromo or iodo, Q² is hydrogen, methyl, ethyl, chloro or bromo, and Q³ is hydrogen, fluoro or bromo.

Of particular interest are those compounds of the formula (I), wherein R³ is tert-butyl, 1-halo-2-methylprop-2-yl, 1,1-dihalo-2-methylprop-2-yl, 1,1,1-trihalo-2-methylprop-2-yl, 1-alkoxy-2-methylprop-2-yl, 1-alkenyloxy-2-methylprop-2-yl, 1-alkynyloxy-2-methylprop-2-yl, 1-cyano-2-methyl-prop-2-yl, 1-alkoxyalkoxy-2-methyl-prop-2-yl, 1-halo-3-methylbut-3-yl, 1-alkoxy-3-methylbut-3-yl, 1-alkenyloxy-3-methylbut-3-yl, 1-alkynyloxy-3-methylbut-3-yl, 1-cyano-3-methylbut-3-yl, 2-cyanoprop-2-yl, 2-(C₁₋₆)alkoxycarbonylprop-2-yl, 2-methoxycarbonylprop-2-yl or 2-methylaminocarbonylprop-2-yl, 1-alkylthio-2-methylprop-2-yl, 2-cyano-1-alkoxyprop-2-yl, 1-alkoxy-prop-2-yl, 1-halo-prop-2-yl, 1-cyanoalkyl-3-methylbut-3-yl, 1-haloalkyl-3-methylbut-3-yl, and R¹, R², Q¹, Q² and Q³ are as defined above. Preferably, R¹ is methyl or ethyl, R² is hydrogen, Q¹ is hydrogen or halogen, Q² is hydrogen, C₁₋₄ alkyl or halogen and Q³ is hydrogen or halogen. More preferably, Q¹ is chloro, bromo or iodo, Q² is hydrogen, methyl, ethyl, chloro or bromo, and Q³ is hydrogen, fluoro, chloro or bromo. It is also preferred that R³ is tert-butyl, 1-halo-2-methylprop-2-yl, 1-methoxy-2-methylprop-2-yl, 1-ethoxy-2-methylprop-2-yl, 1-allyloxy-2-methylprop-2-yl, 1-(prop-2-ynyloxy)-2-methylprop-2-yl or 2-cyano-1-methoxyprop-2-yl, and R¹, R², Q¹, Q² and Q³ are as defined above.

Of particular interest are those compounds of the formula (I), wherein R⁴ is C₁₋₆ alkyl optionally substituted with C₁₋₄alkoxy-(C₁₋₄)alkoxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with benzyloxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with C₂₋₆ alkenyloxy, C₂₋₆ alkynyloxy or —S(O)ₓ(C₁₋₆)alkyl, wherein x is 0, 1 or 2 and the alkyl group is optionally substituted with halo, mono- or di-(C₁₋₆)alkylamino, R⁴ is —CH₂—C≡C—R⁵, wherein R⁵ is hydrogen, C₁₋₈ alkyl optionally substituted with halogen, hydroxy, C₁₋₆ alkoxy, C₁₋₃ alkoxy(C₁₋₃)alkoxy, cyano and R¹, R², Q¹, Q² and Q³ are as defined above. Preferably, R¹ is methyl or ethyl, R² is hydrogen, Q¹ is hydrogen or halogen, Q² is hydrogen, C₁₋₄ alkyl or halogen and Q³ is hydrogen or halogen. More preferably, Q¹ is chloro, bromo or iodo, Q² is hydrogen, methyl, ethyl, chloro or bromo, and Q³ is hydrogen, fluoro, chloro or bromo.

Of particular interest are those compounds of the formula (I), wherein the optionally substituted aryl and optionally substituted heteroaryl rings or moieties of the R₅ values are optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)alkenyloxy, C₂₋₆ alkynyloxy, halo(C₂₋₆)alkynyloxy, aryl, aryloxy, aryl(C₁₋₆)alkyl, aryl(C₁₋₆)alkoxy, heteroaryl, heteroaryloxy, heteroaryl(C₁₋₆)alkyl, heteroaryl(C₁₋₆)alkoxy, —SF₅, —S(O)_{g}(C₁₋₄)alkyl wherein g is 0, 1 or 2 and the alkyl is optionally substituted with halo, or R⁵ is optionally substituted with —OSO₂(C₁₋₄)alkyl, wherein the alkyl group is optionally substituted with halo, or R⁵ is optionally substituted with —CONR^{g}R^{h}, —COR^{g}, —CO₂R^{g}, —R^{gg}═NR^{h}, —NR^{g}R^{h}, —NR^{g}COR^{h}, —NR^{g}CO₂R^{h}, —SO₂NR^{g}R^{h} or —NR^{g}SO₂Rⁱ, wherein Rⁱ is C₁₋₆ alkyl optionally substituted with halogen and R^{gg} is C₁₋₆ alkylene, R^{g} and R^{h}, independently of each other, are hydrogen or C₁₋₆ alkyl optionally substituted with halogen, or, in the case of —CONR^{g}R^{h} or —SO₂NR^{g}R^{h}, R^{g}R^{h} may join to form a 5- or 6-membered carbocyclic or heterocyclic ring containing a heteroatom selected from sulphur, oxygen or NR⁰, wherein R⁰ is hydrogen or optionally substituted C₁₋₆alkyl and R¹, R², Q¹, Q² and Q³ are as defined above. Preferably, R¹ is methyl or ethyl, R² is hydrogen, Q¹ is hydrogen or halogen, Q² is hydrogen, C₁₋₄ alkyl or halogen and Q³ is hydrogen or halogen. More preferably, Q¹ is chloro, bromo or iodo, Q² is hydrogen, methyl, ethyl, chloro or bromo, and Q³ is hydrogen, fluoro, chloro or bromo.

Of particular interest are those compounds of the formula (I), wherein the optionally substituted aryl, optionally substituted heteroaryl or optionally substituted 5- to 8-membered ring R⁴ is optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)-alkenyloxy, C₂₋₆ alkynyloxy, halo(C₂₋₆)alkynyloxy, —SF₅, —S(O)ₓ(C₁₋₆)alkyl, wherein x is 0, 1 or 2 and the alkyl group is optionally substituted with halo, or R⁴ is optionally substituted with —OSO₂(C₁₋₄)alkyl, wherein the alkyl group is optionally substituted with halogen, —CONR^{x}R^{y}, —CON(OR^{x})R^{y}, —COR^{x}, —CO₂R^{x}, —CR^{x}═NR^{y}, —NR^{x}R^{y}, —NR^{x}COR^{y}, —NR^{x}CO₂R^{y}, —SO₂NR^{x}R^{y} or —NR^{x}SO₂R^{z}, wherein R^{z} is C₁₋₆ alkyl optionally substituted with halogen and R^{x} and R^{y}, independently of each other, are hydrogen or C₁₋₆ alkyl optionally substituted with halogen and R¹, R², Q¹, Q² and Q³ are as defined above. Preferably, R¹ is methyl or ethyl, R² is hydrogen, Q¹ is hydrogen or halogen, Q² is hydrogen, C₁₋₄ alkyl or halogen and Q³ is hydrogen or halogen. More preferably, Q¹ is chloro, bromo or iodo, Q² is hydrogen, methyl, ethyl, chloro or bromo, and Q³ is hydrogen, fluoro, chloro or bromo.

Of particular interest are those compounds of the formula (I), wherein L is oxygen.

Of particular interest are those compounds of the formula (I), wherein m is 0.

Of particular interest are those compounds of the formula (I), wherein m is 1.

Compounds that form part of the invention are illustrated in Tables 1 to 160 below.

### Table 1

The compounds in Table 1 are of the general formula (I) where Q1, Q2, Q3, Q4, Q5 and Q6 are hydrogen, m is 0, L is O, R¹ is methyl, and R² and R³ have the values given in the Table.

| Compound No. | R² | R³ |
|---|---|---|
| 1 | H | CH₃ |
| 2 | CH₃ | CH₃ |
| 3 | H | C₂H₅ |
| 4 | C₂H₅ | C₂H₅ |
| 5 | H | prop-2-yl |
| 6 | CH₃ | prop-2-yl |
| 7 | prop-2-yl | prop-2-yl |
| 8 | CH₃ | n-butyl |
| 9 | H | but-2-yl |
| 10 | H | 2-methyl-prop-1-yl |
| 11 | 2-methyl-prop-1-yl | 2-methyl-prop-1-yl |
| 12 | H | tert-C₄H₉ |
| 13 | CH₃ | tert-C₄H₉ |
| 14 | H | pent-2-yl |
| 15 | H | pent-3-yl |
| 16 | H | 2-methyl-but-2-yl |
| 17 | H | 3-methyl-but-1-yl |
| 18 | H | 3-methyl-pent-2-yl |
| 19 | H | 4-methyl-pent-2-yl |
| 20 | H | 3,3-dimethyl-but-2-yl |
| 21 | H | 2-methyl-hex-2-yl |
| 22 | H | 2,4-dimethyl-pent-2-yl |
| 23 | H | 2,4,4-trimethyl-but-2-yl |
| 24 | H | 2,4,4-trimethyl-pent-2-yl |
| 25 | H | Cl-n-C₃H₆— |
| 26 | H | Cl—CH₂(CH₃)₂C— |
| 27 | H | F₃C(CH₃)₂C— |
| 28 | H | NC—CH₂— |
| 29 | CH₃ | NC—CH₂— |
| 30 | NC—CH₂— | NC—CH₂— |
| 31 | H | (NC)₂CH— |
| 32 | H | NC—C₂H₄— |
| 33 | CH₃ | NC—C₂H₄— |
| 34 | NC—C₂H₄— | NC—C₂H₄— |
| 35 | H | (CH₃)₂C(CN)— |
| 36 | H | C₂H₅(CH₃)C(CN)— |
| 37 | H | (C₂H₅)₂C(CN)— |
| 38 | H | (CH₃)₂CH(CH₃)C(CN)— |
| 39 | H | HO—CH₂(CH₃)₂C— |
| 40 | H | HO—C₂H₄(CH₃)₂C— |
| 41 | H | 1-hydroxy-2-(hydroxymethyl)-prop-2- |
| | | yl |
| 42 | H | 1-hydroxy-2-(methoxymethyl)prop-2- |
| | | yl |
| 43 | H | 1-methoxy-2-(methoxymethyl)prop- |
| | | 2-yl |
| 44 | H | 1-hydroxy-2-(hydroxymethyl)-but-2-yl |
| 45 | C₂H₅OC₂H₄— | C₂H₅OC₂H₄— |
| 46 | CH₃ | (CH₃O)₂CHCH₂— |
| 47 | H | CH₃O—CH₂(CH₃)₂C— |
| 48 | H | CH₃O—C₂H₄(CH₃)₂C— |
| 49 | H | C₂H₅O—C₂H₄(CH₃)₂C— |
| 50 | H | CH₃S—CH₂(CH₃)₂C— |
| 51 | H | NC—(CH₃O)CH— |
| 52 | H | CH₃OCH₂(CH₃)C(CN)— |
| 53 | H | CH₃SCH₂(CH₃)C(CN)— |
| 54 | H | CH₃(CO)(CH₃)₂C— |
| 55 | H | CH₃CHBr(CO)(CH₃)₂C— |
| 56 | H | CH₃(CO)(OH)CH(CH₃)₂C— |
| 57 | H | CH₃OC₂H₄(CO)(CH₃)₂C— |
| 58 | H | CH₃(CO)CH₂(CH₃)₂C— |
| 59 | H | CH₃O(CO)(CH₃)CH— |
| 60 | H | CH₃O(CO)(CH₃)₂C— |
| 61 | H | C₂H₅O(CO)C₂H₄— |
| 62 | H | CH₃NH(CO)(CH₃)₂C— |
| 63 | H | (CH₃)₂N(CO)(CH₃)₂C— |
| 64 | H | (CH₃)₃SiCH₂— |
| 65 | H | tert-C₄H₉(CH₃)₂SiO—CH₂(CH₃)₂C— |
| 66 | H | tert-C₄H₉(CH₃)₂SiO—C₂H₄(CH₃)₂C— |
| 67 | H | 4-FPhCH₂OCH₂(CH₃)₂C— |
| 68 | H | C₂H₅OCH₂(CH₃)₂C— |
| 69 | H | CH₃OCH₂CH₂OCH₂(CH₃)₂C— |
| 70 | H | CH₂═CHCH₂— |
| 71 | CH₂═CHCH₂— | CH₂═CHCH₂— |
| 72 | H | CH₂═C(CH₃)CH₂— |
| 73 | H | CH₂═CH(CH₃)CH— |
| 74 | H | CH₂═CH(CH₃)₂C— |
| 75 | H | CH₃(CO)CH═CH— |
| 76 | CH₃ | CH₃(CO)CH═CH— |
| 77 | H | pent-3-en-2-yl |
| 78 | H | 2-methyl-hex-3-en-2-yl (E) |
| 79 | H | 2-methyl-hex-3-en-2-yl (Z) |
| 80 | H | 2-methyl-pent-4-en-3-on-2-yl |
| 81 | H | CH₃O(CO)CH═(Cl)C(CH₃)₂C— |
| 82 | H | C₆H₅—C(CH₃)═CH(CH₃)₂C— |
| 83 | H | HC≡CCH₂CH₂— |
| 84 | H | HC≡CCH₂CH(CH₃)— |
| 85 | H | HC≡CCH₂C(CH₃)₂— |
| 86 | H | cyclopropyl |
| 87 | NC—C₂H₄— | cyclopropyl |
| 88 | cycloprop-1-yl | cyclopropyl |
| 89 | H | 1-cyano-cycloprop-1-yl |
| 90 | H | 2-cyano-cycloprop-1-yl |
| 91 | H | 1-methoxycarbonyl-cycloprop-1-yl |
| 92 | H | 1-[N,N-dimethylaminocarbonyl]- |
| | | cycloprop-1-yl |
| 93 | H | 1-[N-methyl-N-methoxy- |
| | | aminocarbonyl]-cycloprop-1-yl |
| 94 | H | 1-cyano-1-cyclopropyl-eth-1-yl |
| 95 | H | cyclopentyl |
| 96 | H | 1-cyano-cyclopent-1-yl |
| 97 | H | cyclohexyl |
| 98 | CH₂═CHCH₂— | cyclohexyl |
| 99 | H | 4-cyano-cyclohex-1-yl |
| 100 | H | 1-cyano-4-methyl-cyclohex-1-yl |
| 101 | H | 4-tert-butyl-1-cyano-cyclohex-1-yl |
| 102 | H | 2-methyl-3-cyanotetrahydro- |
| | | furan-3-yl |
| 103 | H | 5-methyl-1,3-dioxolan-5-yl |
| 104 | H | 5-ethyl-1,3-dioxolan-5-yl |
| 105 | H | 3,5-dimethyl-1,3-dioxolan-5-yl |
| 106 | H | N-ethoxycarbonyl-piperid-4-yl |
| 107 | H | morpholino |
| 108 | H | cyclohex-1-yl-methyl |
| 109 | H | 4-cyano-cyclopenten-3-yl |
| 110 | H | 5-tert-butyl-2H-1,3,4-thiadiazin-2-yl |
| 111 | H | 2-(cyclohexen-1-yl)-eth-1-yl |
| 112 | H | fur-2-yl |
| 113 | H | 5-methoxycarbonyl-fur-2-yl |
| 114 | H | thien-2-yl |
| 115 | H | 2-methoxycarbonyl-thien-3-yl |
| 116 | H | 4-methoxycarbonyl-thien-3-yl |
| 117 | H | oxazol-2-yl |
| 118 | H | 5-methyl-isoxazol-3-yl |
| 119 | H | 4-cyano-3-methyl-isoxazol-5-yl |
| 120 | H | thiazol-2-yl |
| 121 | H | 5-ethylthio-1,3,4-thiadiazol-2-yl |
| 122 | H | fur-2-ylmethyl |
| 123 | H | cyanofur-1-ylmethyl |
| 124 | H | thien-2-ylmethyl |
| 150 | H | C₆H₅CH₂— |
| 151 | CH₃ | C₆H₅CH₂— |
| 152 | H | 2-F—C₆H₄CH₂— |
| 153 | H | 2-Cl—C₆H₄CH₂— |
| 154 | CH₃ | 2-Cl—C₆H₄CH₂— |
| 155 | H | 2-NO₂—C₆H₄CH₂— |
| 156 | H | 2-CH₃—C₆H₄CH₂— |
| 157 | H | 2-CH₃O—C₆H₄CH₂— |
| 158 | H | 2-CHF₂O—C₆H₄CH₂— |
| 159 | H | 2-CH₃S—C₆H₄CH₂— |
| 160 | H | 2-CF₃S—C₆H₄CH₂— |
| 161 | H | 3-Cl—C₆H₄CH₂— |
| 162 | H | 3-I—C₆H₄CH₂— |
| 163 | H | 3-CH₃—C₆H₄CH₂— |
| 164 | H | 3-CH₃O—C₆H₄CH₂— |
| 165 | H | 4-F—C₆H₄CH₂— |
| 166 | H | 4-Cl—C₆H₄CH₂— |
| 167 | H | 4-CH₃—C₆H₄CH₂— |
| 168 | H | 4-CF₃—C₆H₄CH₂— |
| 169 | H | 4-CH₃O—C₆H₄CH₂— |
| 170 | H | 4-CF₃O—C₆H₄CH₂— |
| 171 | H | 2,6-di-F—C₆H₃CH₂— |
| 172 | 3-methyl-but-2-en-1-yl | 2,5-di-F—C₆H₃CH₂— |
| 173 | H | 2-F-4-Cl—C₆H₃CH₂— |
| 174 | H | 2-F-6-Cl—C₆H₃CH₂— |
| 175 | H | 2,6-di-Cl—C₆H₃CH₂— |
| 176 | 4-methyl-pent-2-en-1-yl | 3,4-di-Cl—C₆H₃CH₂— |
| 177 | H | 2-F-6-CH₃O—C₆H₃CH₂— |
| 178 | H | 2,4,5-tri-F—C₆H₂CH₂— |
| 179 | H | 2,4-di-Cl-6-CH₃—C₆H₂CH₂— |
| 180 | H | 3,4,5-tri-CH₃O—C₆H₂CH₂— |
| 181 | H | C₆H₅—CH(CH₃)— |
| 182 | H | 4-F—C₆H₄—CH(CH₃)— |
| 183 | H | 4-NO₂—C₆H₄—CH(CH₃)— |
| 184 | H | 4-n-pentyl-C₆H₄—CH(CH₃)— |
| 185 | H | 4-CH₃SO₂—C₆H₄—CH(CH₃)— |
| 186 | H | C₆H₅(CO)CH₂— |
| 187 | H | C₆H₅—CH(CN)— |
| 188 | H | C₆H₅—(CH₃O)CH— |
| 189 | H | C₆H₅—(CH₃)₂C— |
| 190 | H | m-Cl—C₆H₅—(CH₃)₂C— |
| 191 | H | 3,5-di-Cl—C₆H₃—(CH₃)₂C— |
| 192 | H | C₆H₅—(C₂H₅O(CO))CH— |
| 193 | H | phenethyl |
| 194 | H | 3-methoxy-4-propargyloxy-phenethyl |
| 195 | H | 3-methoxy-4-(pent-2-yn-1-yloxy)- |
| | | phenethyl |
| 196 | H | 2-methyl-3-phenyl-prop-2-yl |
| 197 | H | C₆H₅O—C₂H₄— |
| 198 | H | 4-F—C₆H₄—CH₂OCH₂(CH₃)₂C— |
| 199 | H | C₆H₅—CH₂O(CO)C₂H₄— |
| 200 | H | naphth-2-yl-(CH₃)CH— |
| 201 | NC—C₂H₄— | pyrid-3-ylmethyl |
| 202 | CH₃ | 2-pyrid-2-yleth-1-yl |
| 203 | H | 2-(3-chloro-5-trifluoromethyl-pyrid-2- |
| | | yl)oxyeth-1-yl |
| 204 | H | 2-methyl-4-pyrazin-2-yl-but-3-on-2-yl |
| 205 | —(CH₂)₄— | |
| 206 | —(CH₂)₅— | |
| 207 | —(CH₂)₄CH(C₂H₅)— | |
| 208 | —C₃H₆CH[(CO)N(C₂H₅)₂]CH₂— | |
| 209 | —CH(CH₃)CH═CHCH(CH₃)— | |
| 210 | 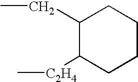 | |
| 211 | —C₂H₄OC₂H₄— | |
| 212 | —CH₂CH(CH₃)OCH(CH₃)CH₂— | |
| 213 | —C₂H₄SCH₂— | |
| 214 | —C₂H₄SC₂H₄— | |
| 215 | —(CH₂)₂NH(CH₂)₂— | |
| 216 | —(CH₂)₂N(p-NO₂—C₆H₄(CH₂)₂— | |
| 217 | —(CH₂)₂N(m-CF₃—C₆H₄(CH₂)₂— | |
| 218 | —(CH₂)₂N(p-CH₃CO—C₆H₄)(CH₂)₂— | |
| 219 | —(CH₂)₂N(pyrid-2-yl)(CH₂)₂— | |
| 220 | H | (H₂C═CHCH₂OCH₂)(CH₃)₂C— |
| 221 | H | (HCCHCH₂OCH₂)(CH₃)₂C— |
| 222 | H | (CH₃CH₂OCH₂)(CH₃)₂C— |
| 223 | H | ((CH₃)₂CHOCH₂)(CH₃)₂C— |
| 224 | H | C₆H₅CH₂OCH₂(CH₃)₂C— |
| 225 | H | (CH₃CH₂OCH₂)(CH₃)₂C— |
| 226 | H | 4-F—C₆H₄—CH₂(CH₃)C(CN)— |
| 227 | H | 4-Cl—C₆H₄—CH₂(CH₃)C(CN)— |
| 228 | H | 4-CH₃O—C₆H₄—CH₂CH₂(CH₃)C(CN)— |
| 229 | H | 2-Cl—C₆H₄—CH₂(CH₃)C(CN)— |
| 230 | H | (CH₃)₂CH—CH₂(CH₃)C(CN)— |
| 231 | H | 1-methoxymethyl-cycloprop-1-yl |
| 232 | H | 1-benzyloxymethyl-cycloprop-1-yl |
| 233 | H | 1-methoxymethoxy-2-methyl-prop-2- |
| | | yl |
| 235 | H | 1-cyclopropyl-eth-1-yl |
| 236 | H | 2-fluoro-eth-1-yl |
| 237 | H | 2,2,2-trifluoro-1-methyl-eth-1-yl |
| 261 | H | (H₂C═CHCH₂OCH₂)(CH₃)CH— |
| 262 | H | (HC≡CHCH₂OCH₂)(CH₃)CH— |
| 263 | H | (CH₃CH₂OCH₂)(CH₃)CH— |
| 264 | H | (CH₃OCH₂)(CH₃)CH— |
| 265 | H | ((CH₃)₂CHOCH₂)(CH₃)CH— |
| 266 | H | C₆H₅CH₂OCH₂(CH₃)CH— |
| 267 | H | (CH₃CH₂OCH₂)(CH₃)CH— |
| 268 | H | (cC₄H₇)CH2— |
| 269 | H | (cC₄H₇)CH₃CH— |
| 270 | H | FCH₂(CH₃)CH— |
| 271 | H | ClCH₂(CH₃)CH— |
| 272 | H | FCH₂CH₂(CH₃)CH— |
| 273 | H | ClCH₂CH₂(CH₃)CH— |
| 274 | H | FCH₂(CH₃)₂C— |
| 275 | H | FCH₂CH₂(CH₃)₂C— |
| 276 | H | ClCH₂CH₂(CH₃)₂C— |
| 278 | H | tetrahydro-furan-2-ylmethyl |
| 279 | H | 1-(tetrahydro-furan-2-yl)ethyl |
| 280 | H | 1-methyl-1-(tetrahydro-furan-2-yl)ethyl |
| 281 | H | 2-[1,3]dioxolan-2-yl-ethyl |
| 282 | H | 2-[1,3]dioxolan-2-yl-1-methyl-ethyl |
| 283 | H | 2-[1,3]dioxolan-2-yl-1,1-dimethyl-ethyl |
| 284 | H | prop-1-yl |
| 285 | CH₃ | prop-1-yl |
| 286 | H | thiophen-3-ylmethyl |
| 287 | H | 1-(thiophen-3-yl)-eth-1-yl |
| 289 | H | 1-methyl-1-(thiophen-3-yl)-eth-1-yl |
| 290 | H | cyclobutyl |
| 291 | H | 1-methyl-cyclobut-1-yl |
| 292 | H | 3-F—C₆H₄—CH(CH₃)— |
| 293 | H | 3-F—C₆H₄—C(CH₃)₂— |
| 294 | H | 1-cyano-cyclobut-1-yl |
| 295 | H | (O)HC(CH₃)₂C— |
| 296 | H | (MeO)₂C(CH₃)₂C— |
| 297 | H | (EtO)₂C(CH₃)₂C— |
| 298 | H | (O(CH₂)₂O)C(CH₃)₂C— |
| 299 | H | (O(CH₂)₃O)C(CH₃)₂C— |

### Table 2

The compounds in Table 2 are of the general formula (I) where Q1, Q2, Q3, Q4, Q5 & Q6 are hydrogen, m is 0, L is O, R¹ is ethyl, and R² and R³ have the values given in the table. Thus, compound 1 of Table 2 is the same as compound 1 of Table 1 except that in compound 1 of Table 2, R¹ is ethyl. Similarly, compounds 2 to 299 of Table 2 are the same as compounds 2 to 299 of Table 1, respectively, except that in the compounds of Table 2, R¹ is ethyl.

### Table 3

The compounds in Table 3 are of the general formula (I) where Q1, Q2, Q3, Q4, Q5 & Q6 are hydrogen, m is 1, L is O, R¹ is methyl, and R² and R³ have the values given in the table. Thus, compound 1 of Table 3 is the same as compound 1 of Table 1 except that in compound 1 of Table 3, m is 1. Similarly, compounds 2 to 299 of Table 3 are the same as compounds 2 to 299 of Table 1, respectively, except that in the compounds of Table 3, m is 1.

### Table 4

The compounds in Table 4 are of the general formula (I) where Q1, Q2, Q3, Q4, Q5 & Q6 are hydrogen, m is 1, L is O, R¹ is ethyl, and R² and R³ have the values given in the table. Thus, compound 1 of Table 4 is the same as compound 1 of Table 1 except that in compound 1 of Table 4, m is 1, R¹ is ethyl. Similarly, compounds 2 to 299 of Table 4 are the same as compounds 2 to 299 of Table 1, respectively, except that in the compounds of Table 4, m is 1, R¹ is ethyl.

### Table 5

The compounds in Table 5 are of the general formula (I) where Q1, Q3, Q4, Q5 & Q6 are hydrogen, Q2 is methyl, m is 0, L is O, R¹ is methyl, and R² and R³ have the values given in the table. Thus, compound 1 of Table 5 is the same as compound 1 of Table 1 except that in compound 1 of Table 5, Q2 is methyl. Similarly, compounds 2 to 299 of Table 5 are the same as compounds 2 to 299 of Table 1, respectively, except that in the compounds of Table 4, Q2 is methyl.

### Table 6

The compounds in Table 6 are of the general formula (I) where Q1, Q3, Q4, Q5 & Q6 are hydrogen, Q2 is methyl, m is 0, L is O, R¹ is ethyl, and R² and R³ have the values given in the table. Thus, compound 1 of Table 6 is the same as compound 1 of Table 1 except that in compound 1 of Table 6, Q2 is methyl, R¹ is ethyl. Similarly, compounds 2 to 299 of Table 6 are the same as compounds 2 to 299 of Table 1, respectively, except that in the compounds of Table 6, Q2 is methyl, R¹ is ethyl.

### Table 7

The compounds in Table 7 are of the general formula (I) where Q1, Q3, Q4, Q5 & Q6 are hydrogen, Q2 is methyl, m is 1, L is O, R¹ is methyl, and R² and R³ have the values given in the table. Thus, compound 1 of Table 7 is the same as compound 1 of Table 1 except that in compound 1 of Table 7, Q2 is methyl, m is 1. Similarly, compounds 2 to 299 of Table 7 are the same as compounds 2 to 299 of Table 1, respectively, except that in the compounds of Table 7, Q2 is methyl, m is 1.

### Table 8

The compounds in Table 8 are of the general formula (I) where Q1, Q3, Q4, Q5 & Q6 are hydrogen, Q2 is methyl, m is 1, L is O, R¹ is ethyl, and R² and R³ have the values given in the table. Thus, compound 1 of Table 8 is the same as compound 1 of Table 1 except that in compound 1 of Table 8, Q2 is methyl, m is 1, R¹ is ethyl. Similarly, compounds 2 to 299 of Table 8 are the same as compounds 2 to 299 of Table 1, respectively, except that in the compounds of Table 8, Q2 is methyl, m is 1, R¹ is ethyl.

### Table 9

The compounds in Table 9 are of the general formula (I) where Q1, Q3, Q4, Q5 & Q6 are hydrogen, Q2 is chloro, m is 0, L is O, R¹ is methyl, and R² and R³ have the values given in the table. Thus, compound 1 of Table 9 is the same as compound 1 of Table 1 except that in compound 1 of Table 9, Q2 is chloro. Similarly, compounds 2 to 299 of Table 9 are the same as compounds 2 to 299 of Table 1, respectively, except that in the compounds of Table 9, Q2 is chloro.

### Table 10

The compounds in Table 10 are of the general formula (I) where Q1, Q3, Q4, Q5 & Q6 are hydrogen, Q2 is chloro, m is 0, L is O, R¹ is ethyl, and R² and R³ have the values given in the table. Thus, compound 1 of Table 10 is the same as compound 1 of Table 1 except that in compound 1 of Table 10, Q2 is chloro, R¹ is ethyl. Similarly, compounds 2 to 299 of Table 10 are the same as compounds 2 to 299 of Table 1, respectively, except that in the compounds of Table 10, Q2 is chloro, R¹ is ethyl.

### Table 11

The compounds in Table 11 are of the general formula (I) where Q1, Q3, Q4, Q5 & Q6 are hydrogen, Q2 is chloro, m is 1, L is O, R¹ is methyl, and R² and R³ have the values given in the table. Thus, compound 1 of Table 11 is the same as compound 1 of Table 1 except that in compound 1 of Table 11, Q2 is chloro, m is 1. Similarly, compounds 2 to 299 of Table 11 are the same as compounds 2 to 299 of Table 1, respectively, except that in the compounds of Table 11, Q2 is chloro, m is 1.

### Table 12

The compounds in Table 12 are of the general formula (I) where Q1, Q3, Q4, Q5 & Q6 are hydrogen, Q2 is chloro, m is 1, L is O, R¹ is ethyl, and R² and R³ have the values given in the table. Thus, compound 1 of Table 12 is the same as compound 1 of Table 1 except that in compound 1 of Table 12, Q2 is chloro, m is 1, R¹ is ethyl. Similarly, compounds 2 to 299 of Table 12 are the same as compounds 2 to 299 of Table 1, respectively, except that in the compounds of Table 12, Q2 is chloro, m is 1, R¹ is ethyl.

### Table 13

The compounds in Table 13 are of the general formula (I) where Q1, Q3, Q4, Q5 & Q6 are hydrogen, Q2 is bromine, m is 0, L is O, R¹ is methyl, and R² and R³ have the values given in the table. Thus, compound 1 of Table 13 is the same as compound 1 of Table 1 except that in compound 1 of Table 13, Q2 is bromine. Similarly, compounds 2 to 299 of Table 13 are the same as compounds 2 to 299 of Table 1, respectively, except that in the compounds of Table 13, Q2 is bromine.

### Table 14

The compounds in Table 14 are of the general formula (I) where Q1, Q3, Q4, Q5 & Q6 are hydrogen, Q2 is bromine, m is 0, L is O, R¹ is ethyl, and R² and R³ have the values given in the table. Thus, compound 1 of Table 14 is the same as compound 1 of Table 1 except that in compound 1 of Table 14, Q2 is bromine, R¹ is ethyl. Similarly, compounds 2 to 299 of Table 14 are the same as compounds 2 to 299 of Table 1, respectively, except that in the compounds of Table 14, Q2 is bromine, R¹ is ethyl.

### Table 15

The compounds in Table 15 are of the general formula (I) where Q1, Q3, Q4, Q5 & Q6 are hydrogen, Q2 is bromine, m is 1, L is O, R¹ is methyl, and R² and R³ have the values given in the table. Thus, compound 1 of Table 15 is the same as compound 1 of Table 1 except that in compound 1 of Table 15, Q2 is bromine, m is 1. Similarly, compounds 2 to 299 of Table 15 are the same as compounds 2 to 299 of Table 1, respectively, except that in the compounds of Table 15, Q2 is bromine, m is 1.

### Table 16

The compounds in Table 16 are of the general formula (I) where Q1, Q3, Q4, Q5 & Q6 are hydrogen, Q2 is bromine, m is 1, L is O, R¹ is ethyl, and R² and R³ have the values given in the table. Thus, compound 1 of Table 16 is the same as compound 1 of Table 1 except that in compound 1 of Table 16, Q2 is bromine, m is 1, R¹ is ethyl. Similarly, compounds 2 to 299 of Table 16 are the same as compounds 2 to 299 of Table 1, respectively, except that in the compounds of Table 16, Q2 is bromine, m is 1, R¹ is ethyl.

### Table 17

The compounds in Table 17 are of the general formula (I) where Q1, Q2, Q4, Q5 & Q6 are hydrogen, Q3 is fluoro, m is 0, L is O, R¹ is methyl, and R² and R³ have the values given in the table. Thus, compound 1 of Table 17 is the same as compound 1 of Table 1 except that in compound 1 of Table 17, Q3 is fluoro. Similarly, compounds 2 to 299 of Table 17 are the same as compounds 2 to 299 of Table 1, respectively, except that in the compounds of Table 17, Q3 is fluoro.

### Table 18

The compounds in Table 18 are of the general formula (I) where Q1, Q2, Q4, Q5 & Q6 are hydrogen, Q3 is fluoro, m is 0, L is O, R¹ is ethyl, and R² and R³ have the values given in the table. Thus, compound 1 of Table 18 is the same as compound 1 of Table 1 except that in compound 1 of Table 18, Q3 is fluoro, R¹ is ethyl. Similarly, compounds 2 to 299 of Table 18 are the same as compounds 2 to 299 of Table 1, respectively, except that in the compounds of Table 18, Q3 is fluoro, R¹ is ethyl.

### Table 19

The compounds in Table 19 are of the general formula (I) where Q1, Q2, Q4, Q5 & Q6 are hydrogen, Q3 is fluoro, m is 1, L is O, R¹ is methyl, and R² and R³ have the values given in the table. Thus, compound 1 of Table 19 is the same as compound 1 of Table 1 except that in compound 1 of Table 19, Q3 is fluoro, m is 1. Similarly, compounds 2 to 299 of Table 19 are the same as compounds 2 to 299 of Table 1, respectively, except that in the compounds of Table 19, Q3 is fluoro, m is 1.

### Table 20

The compounds in Table 20 are of the general formula (I) where Q1, Q2, Q4, Q5 & Q6 are hydrogen, Q3 is fluoro, m is 1, L is O, R¹ is ethyl, and R² and R³ have the values given in the table. Thus, compound 1 of Table 20 is the same as compound 1 of Table 1 except that in compound 1 of Table 20, Q3 is fluoro, m is 1, R¹ is ethyl. Similarly, compounds 2 to 299 of Table 20 are the same as compounds 2 to 299 of Table 1, respectively, except that in the compounds of Table 20, Q3 is fluoro, m is 1, R¹ is ethyl.

### Table 21

The compounds in Table 21 are of the general formula (I) where Q1, Q2, Q4, Q5 & Q6 are hydrogen, Q3 is chloro, m is 0, L is O, R¹ is methyl, and R² and R³ have the values given in the table. Thus, compound 1 of Table 21 is the same as compound 1 of Table 1 except that in compound 1 of Table 21, Q3 is chloro. Similarly, compounds 2 to 299 of Table 21 are the same as compounds 2 to 299 of Table 1, respectively, except that in the compounds of Table 21, Q3 is chloro.

### Table 22

The compounds in Table 22 are of the general formula (I) where Q1, Q2, Q4, Q5 & Q6 are hydrogen, Q3 is chloro, m is 0, L is O, R¹ is ethyl, and R² and R³ have the values given in the table. Thus, compound 1 of Table 22 is the same as compound 1 of Table 1 except that in compound 1 of Table 22, Q3 is chloro, R¹ is ethyl. Similarly, compounds 2 to 299 of Table 22 are the same as compounds 2 to 299 of Table 1, respectively, except that in the compounds of Table 22, Q3 is chloro, R¹ is ethyl.

### Table 23

The compounds in Table 23 are of the general formula (I) where Q1, Q2, Q4, Q5 & Q6 are hydrogen, Q3 is chloro, m is 1, L is O, R¹ is methyl, and R² and R³ have the values given in the table. Thus, compound 1 of Table 23 is the same as compound 1 of Table 1 except that in compound 1 of Table 23, Q3 is chloro, m is 1. Similarly, compounds 2 to 299 of Table 23 are the same as compounds 2 to 299 of Table 1, respectively, except that in the compounds of Table 23, Q3 is chloro, m is 1.

### Table 24

The compounds in Table 24 are of the general formula (I) where Q1, Q2, Q4, Q5 & Q6 are hydrogen, Q3 is chloro, m is 1, L is O, R¹ is ethyl, and R² and R³ have the values given in the table. Thus, compound 1 of Table 24 is the same as compound 1 of Table 1 except that in compound 1 of Table 24, Q3 is chloro, m is 1, R¹ is ethyl. Similarly, compounds 2 to 299 of Table 24 are the same as compounds 2 to 299 of Table 1, respectively, except that in the compounds of Table 24, Q3 is chloro, m is 1, R¹ is ethyl.

### Table 25

The compounds in Table 25 are of the general formula (I) where Q1, Q2, Q4, Q5 & Q6 are hydrogen, Q3 is bromine, m is 0, L is O, R¹ is methyl, and R² and R³ have the values given in the table. Thus, compound 1 of Table 25 is the same as compound 1 of Table 1 except that in compound 1 of Table 25, Q3 is bromine. Similarly, compounds 2 to 299 of Table 25 are the same as compounds 2 to 299 of Table 1, respectively, except that in the compounds of Table 25, Q3 is bromine.

### Table 26

The compounds in Table 26 are of the general formula (I) where Q1, Q2, Q4, Q5 & Q6 are hydrogen, Q3 is bromine, m is 0, L is O, R¹ is ethyl, and R² and R³ have the values given in the table. Thus, compound 1 of Table 26 is the same as compound 1 of Table 1 except that in compound 1 of Table 26, Q3 is bromine, R¹ is ethyl. Similarly, compounds 2 to 299 of Table 26 are the same as compounds 2 to 299 of Table 1, respectively, except that in the compounds of Table 26, Q3 is bromine, R¹ is ethyl.

### Table 27

The compounds in Table 27 are of the general formula (I) where Q1, Q2, Q4, Q5 & Q6 are hydrogen, Q3 is bromine, m is 1, L is O, R¹ is methyl, and R² and R³ have the values given in the table. Thus, compound 1 of Table 27 is the same as compound 1 of Table 1 except that in compound 1 of Table 27, Q3 is bromine, m is 1. Similarly, compounds 2 to 299 of Table 27 are the same as compounds 2 to 299 of Table 1, respectively, except that in the compounds of Table 27, Q3 is bromine, m is 1.

### Table 28

The compounds in Table 28 are of the general formula (I) where Q1, Q2, Q4, Q5 & Q6 are hydrogen, Q3 is bromine, m is 1, L is O, R¹ is ethyl, and R² and R³ have the values given in the table. Thus, compound 1 of Table 28 is the same as compound 1 of Table 1 except that in compound 1 of Table 28, Q3 is bromine, m is 1, R¹ is ethyl. Similarly, compounds 2 to 299 of Table 28 are the same as compounds 2 to 299 of Table 1, respectively, except that in the compounds of Table 28, Q3 is bromine, m is 1, R¹ is ethyl.

### Table 29

The compounds in Table 29 are of the general formula (I) where Q1, Q2, Q4, Q5 & Q6 are hydrogen, Q3 is methyl, m is 0, L is O, R¹ is methyl, and R² and R³ have the values given in the table. Thus, compound 1 of Table 29 is the same as compound 1 of Table 1 except that in compound 1 of Table 29, Q3 is methyl. Similarly, compounds 2 to 299 of Table 29 are the same as compounds 2 to 299 of Table 1, respectively, except that in the compounds of Table 29, Q3 is methyl.

### Table 30

The compounds in Table 30 are of the general formula (I) where Q1, Q2, Q4, Q5 & Q6 are hydrogen, Q3 is methyl, m is 0, L is O, R¹ is ethyl, and R² and R³ have the values given in the table. Thus, compound 1 of Table 30 is the same as compound 1 of Table 1 except that in compound 1 of Table 30, Q3 is methyl, R¹ is ethyl. Similarly, compounds 2 to 299 of Table 30 are the same as compounds 2 to 299 of Table 1, respectively, except that in the compounds of Table 30, Q3 is methyl, R¹ is ethyl.

### Table 31

The compounds in Table 31 are of the general formula (I) where Q1, Q2, Q4, Q5 & Q6 are hydrogen, Q3 is methyl, m is 1, L is O, R¹ is methyl, and R² and R³ have the values given in the table. Thus, compound 1 of Table 31 is the same as compound 1 of Table 1 except that in compound 1 of Table 31, Q3 is methyl, m is 1. Similarly, compounds 2 to 299 of Table 31 are the same as compounds 2 to 299 of Table 1, respectively, except that in the compounds of Table 31, Q3 is methyl, m is 1.

### Table 32

The compounds in Table 32 are of the general formula (I) where Q1, Q2, Q4, Q5 & Q6 are hydrogen, Q3 is methyl, m is 1, L is O, R¹ is ethyl, and R² and R³ have the values given in the table. Thus, compound 1 of Table 32 is the same as compound 1 of Table 1 except that in compound 1 of Table 32, Q3 is methyl, m is 1, R¹ is ethyl. Similarly, compounds 2 to 299 of Table 32 are the same as compounds 2 to 299 of Table 1, respectively, except that in the compounds of Table 32, Q3 is methyl, m is 1, R¹ is ethyl.

### Table 33

The compounds in Table 33 are of the general formula (I) where Q1 is chlorine, Q3, Q4, Q5 & Q6 are hydrogen, Q2 is methyl, m is 0, L is O, R¹ is methyl, and R² and R³ have the values given in the table. Thus, compound 1 of Table 33 is the same as compound 1 of Table 1 except that in compound 1 of Table 33, Q1 is chlorine, Q2 is methyl.

Similarly, compounds 2 to 299 of Table 33 are the same as compounds 2 to 299 of Table 1, respectively, except that in the compounds of Table 33, Q1 is chlorine, Q2 is methyl.

### Table 34

The compounds in Table 34 are of the general formula (I) where Q1 is chlorine, Q3, Q4, Q5 & Q6 are hydrogen, Q2 is methyl, m is 0, L is O, R¹ is ethyl, and R² and R³ have the values given in the table. Thus, compound 1 of Table 34 is the same as compound 1 of Table 1 except that in compound 1 of Table 34, Q1 is chlorine, Q2 is methyl, R¹ is ethyl. Similarly, compounds 2 to 299 of Table 34 are the same as compounds 2 to 299 of Table 1, respectively, except that in the compounds of Table 34, Q1 is chlorine, Q2 is methyl, R¹ is ethyl.

### Table 35

The compounds in Table 35 are of the general formula (I) where Q1 is chlorine, Q3, Q4, Q5 & Q6 are hydrogen, Q2 is methyl, m is 1, L is O, R¹ is methyl, and R² and R³ have the values given in the table. Thus, compound 1 of Table 35 is the same as compound 1 of Table 1 except that in compound 1 of Table 35, Q1 is chlorine, Q2 is methyl, m is 1. Similarly, compounds 2 to 299 of Table 35 are the same as compounds 2 to 299 of Table 1, respectively, except that in the compounds of Table 35, Q1 is chlorine, Q2 is methyl, m is 1.

### Table 36

The compounds in Table 36 are of the general formula (I) where Q1 is chlorine, Q3, Q4, Q5 & Q6 are hydrogen, Q2 is methyl, m is 1, L is O, R¹ is ethyl, and R² and R³ have the values given in the table. Thus, compound 1 of Table 36 is the same as compound 1 of Table 1 except that in compound 1 of Table 36, Q1 is chlorine, Q2 is methyl, m is 1, R¹ is ethyl. Similarly, compounds 2 to 299 of Table 36 are the same as compounds 2 to 299 of Table 1, respectively, except that in the compounds of Table 36, Q1 is chlorine, Q2 is methyl, m is 1, R¹ is ethyl.

### Table 37

The compounds in Table 37 are of the general formula (I) where Q1 is chlorine, Q3, Q4, Q5 & Q6 are hydrogen, Q2 is chlorine, m is 0, L is O, R¹ is methyl, and R² and R³ have the values given in the table. Thus, compound 1 of Table 37 is the same as compound 1 of Table 1 except that in compound 1 of Table 37, Q1 is chlorine, Q2 is chlorine. Similarly, compounds 2 to 299 of Table 37 are the same as compounds 2 to 299 of Table 1, respectively, except that in the compounds of Table 37, Q1 is chlorine, Q2 is chlorine.

### Table 38

The compounds in Table 38 are of the general formula (I) where Q1 is chlorine, Q3, Q4, Q5 & Q6 are hydrogen, Q2 is chlorine, m is 0, L is O, R¹ is ethyl, and R² and R³ have the values given in the table. Thus, compound 1 of Table 38 is the same as compound 1 of Table 1 except that in compound 1 of Table 38, Q1 is chlorine, Q2 is chlorine, R¹ is ethyl. Similarly, compounds 2 to 299 of Table 38 are the same as compounds 2 to 299 of Table 1, respectively, except that in the compounds of Table 38, Q1 is chlorine, Q2 is chlorine, R¹ is ethyl.

### Table 39

The compounds in Table 39 are of the general formula (I) where Q1 is chlorine, Q3, Q4, Q5 & Q6 are hydrogen, Q2 is chlorine, m is 1, L is O, R¹ is methyl, and R² and R³ have the values given in the table. Thus, compound 1 of Table 39 is the same as compound 1 of Table 1 except that in compound 1 of Table 39, Q1 is chlorine, Q2 is chlorine, m is 1. Similarly, compounds 2 to 299 of Table 39 are the same as compounds 2 to 299 of Table 1, respectively, except that in the compounds of Table 39, Q1 is chlorine, Q2 is chlorine, m is 1.

### Table 40

The compounds in Table 40 are of the general formula (I) where Q1 is chlorine, Q3, Q4, Q5 & Q6 are hydrogen, Q2 is chlorine, m is 1, L is O, R¹ is ethyl, and R² and R³ have the values given in the table. Thus, compound 1 of Table 40 is the same as compound 1 of Table 1 except that in compound 1 of Table 40, Q1 is chlorine, Q2 is chlorine, m is 1, R¹ is ethyl. Similarly, compounds 2 to 299 of Table 40 are the same as compounds 2 to 299 of Table 1, respectively, except that in the compounds of Table 40, Q1 is chlorine, Q2 is chlorine, m is 1, R¹ is ethyl.

### Table 41

The compounds in Table 41 are of the general formula (I) where Q1 is chlorine, Q3, Q4, Q5 & Q6 are hydrogen, Q2 is bromine, m is 0, L is O, R¹ is methyl, and R² and R³ have the values given in the table. Thus, compound 1 of Table 41 is the same as compound 1 of Table 1 except that in compound 1 of Table 41, Q1 is chlorine, Q2 is bromine. Similarly, compounds 2 to 299 of Table 41 are the same as compounds 2 to 299 of Table 1, respectively, except that in the compounds of Table 41, Q1 is chlorine, Q2 is bromine.

### Table 42

The compounds in Table 42 are of the general formula (I) where Q1 is chlorine, Q3, Q4, Q5 & Q6 are hydrogen, Q2 is bromine, m is 0, L is O, R¹ is ethyl, and R² and R³ have the values given in the table. Thus, compound 1 of Table 42 is the same as compound 1 of Table 1 except that in compound 1 of Table 42, Q1 is chlorine, Q2 is bromine, R¹ is ethyl. Similarly, compounds 2 to 299 of Table 42 are the same as compounds 2 to 299 of Table 1, respectively, except that in the compounds of Table 42, Q1 is chlorine, Q2 is bromine, R¹ is ethyl.

### Table 43

The compounds in Table 43 are of the general formula (I) where Q1 is chlorine, Q3, Q4, Q5 & Q6 are hydrogen, Q2 is bromine, m is 1, L is O, R¹ is methyl, and R² and R³ have the values given in the table. Thus, compound 1 of Table 43 is the same as compound 1 of Table 1 except that in compound 1 of Table 43, Q1 is chlorine, Q2 is bromine, m is 1. Similarly, compounds 2 to 299 of Table 43 are the same as compounds 2 to 299 of Table 1, respectively, except that in the compounds of Table 43, Q1 is chlorine, Q2 is bromine, m is 1.

### Table 44

The compounds in Table 44 are of the general formula (I) where Q1 is chlorine, Q3, Q4, Q5 & Q6 are hydrogen, Q2 is bromine, m is 1, L is O, R¹ is ethyl, and R² and R³ have the values given in the table. Thus, compound 1 of Table 44 is the same as compound 1 of Table 1 except that in compound 1 of Table 44, Q1 is chlorine, Q2 is bromine, m is 1, R¹ is ethyl. Similarly, compounds 2 to 299 of Table 44 are the same as compounds 2 to 299 of Table 1, respectively, except that in the compounds of Table 44, Q1 is chlorine, Q2 is bromine, m is 1, R¹ is ethyl.

### Table 45

The compounds in Table 45 are of the general formula (I) where Q1 is chlorine, Q2, Q4, Q5 & Q6 are hydrogen, Q3 is fluoro, m is 0, L is O, R¹ is methyl, and R² and R³ have the values given in the table. Thus, compound 1 of Table 45 is the same as compound 1 of Table 1 except that in compound 1 of Table 45, Q1 is chlorine, Q3 is fluoro. Similarly, compounds 2 to 299 of Table 45 are the same as compounds 2 to 299 of Table 1, respectively, except that in the compounds of Table 45, Q1 is chlorine, Q3 is fluoro.

### Table 46

The compounds in Table 46 are of the general formula (I) where Q1 is chlorine, Q2, Q4, Q5 & Q6 are hydrogen, Q3 is fluoro, m is 0, L is O, R¹ is ethyl, and R² and R³ have the values given in the table. Thus, compound 1 of Table 46 is the same as compound 1 of Table 1 except that in compound 1 of Table 46, Q1 is chlorine, Q3 is fluoro, R¹ is ethyl. Similarly, compounds 2 to 299 of Table 46 are the same as compounds 2 to 299 of Table 1, respectively, except that in the compounds of Table 46, Q1 is chlorine, Q3 is fluoro, R¹ is ethyl.

### Table 47

The compounds in Table 47 are of the general formula (I) where Q1 is chlorine, Q2, Q4, Q5 & Q6 are hydrogen, Q3 is fluoro, m is 1, L is O, R¹ is methyl, and R² and R³ have the values given in the table. Thus, compound 1 of Table 47 is the same as compound 1 of Table 1 except that in compound 1 of Table 47, Q1 is chlorine, Q3 is fluoro, m is 1. Similarly, compounds 2 to 299 of Table 47 are the same as compounds 2 to 299 of Table 1, respectively, except that in the compounds of Table 47, Q1 is chlorine, Q3 is fluoro, m is 1.

### Table 48

The compounds in Table 48 are of the general formula (I) where Q1 is chlorine, Q2, Q4, Q5 & Q6 are hydrogen, Q3 is fluoro, m is 1, L is O, R¹ is ethyl, and R² and R³ have the values given in the table. Thus, compound 1 of Table 48 is the same as compound 1 of Table 1 except that in compound 1 of Table 48, Q1 is chlorine, Q3 is fluoro, m is 1, R¹ is ethyl. Similarly, compounds 2 to 299 of Table 48 are the same as compounds 2 to 299 of Table 1, respectively, except that in the compounds of Table 48, Q1 is chlorine, Q3 is fluoro, m is 1, R¹ is ethyl.

### Table 49

The compounds in Table 49 are of the general formula (I) where Q1 is chlorine, Q2, Q4, Q5 & Q6 are hydrogen, Q3 is chlorine, m is 0, L is O, R¹ is methyl, and R² and R³ have the values given in the table. Thus, compound 1 of Table 49 is the same as compound 1 of Table 1 except that in compound 1 of Table 49, Q1 is chlorine, Q3 is chlorine. Similarly, compounds 2 to 299 of Table 49 are the same as compounds 2 to 299 of Table 1, respectively, except that in the compounds of Table 49, Q1 is chlorine, Q3 is chlorine.

### Table 50

The compounds in Table 50 are of the general formula (I) where Q1 is chlorine, Q2, Q4, Q5 & Q6 are hydrogen, Q3 is chlorine, m is 0, L is O, R¹ is ethyl, and R² and R³ have the values given in the table. Thus, compound 1 of Table 50 is the same as compound 1 of Table 1 except that in compound 1 of Table 50, Q1 is chlorine, Q3 is chlorine, R¹ is ethyl. Similarly, compounds 2 to 299 of Table 50 are the same as compounds 2 to 299 of Table 1, respectively, except that in the compounds of Table 50, Q1 is chlorine, Q3 is chlorine, R¹ is ethyl.

### Table 51

The compounds in Table 51 are of the general formula (I) where Q1 is chlorine, Q2, Q4, Q5 & Q6 are hydrogen, Q3 is chlorine, m is 1, L is O, R¹ is methyl, and R² and R³ have the values given in the table. Thus, compound 1 of Table 51 is the same as compound 1 of Table 1 except that in compound 1 of Table 51, Q1 is chlorine, Q3 is chlorine, m is 1. Similarly, compounds 2 to 299 of Table 51 are the same as compounds 2 to 299 of Table 1, respectively, except that in the compounds of Table 51, Q1 is chlorine, Q3 is chlorine, m is 1.

### Table 52

The compounds in Table 52 are of the general formula (I) where Q1 is chlorine, Q2, Q4, Q5 & Q6 are hydrogen, Q3 is chlorine, m is 1, L is O, R¹ is ethyl, and R² and R³ have the values given in the table. Thus, compound 1 of Table 52 is the same as compound 1 of Table 1 except that in compound 1 of Table 52, Q1 is chlorine, Q3 is chlorine, m is 1, R¹ is ethyl. Similarly, compounds 2 to 299 of Table 52 are the same as compounds 2 to 299 of Table 1, respectively, except that in the compounds of Table 52, Q1 is chlorine, Q3 is chlorine, m is 1, R¹ is ethyl.

### Table 53

The compounds in Table 53 are of the general formula (I) where Q1 is chlorine, Q2, Q4, Q5 & Q6 are hydrogen, Q3 is bromine, m is 0, L is O, R¹ is methyl, and R² and R³ have the values given in the table. Thus, compound 1 of Table 53 is the same as compound 1 of Table 1 except that in compound 1 of Table 53, Q1 is chlorine, Q3 is bromine. Similarly, compounds 2 to 299 of Table 53 are the same as compounds 2 to 299 of Table 1, respectively, except that in the compounds of Table 53, Q1 is chlorine, Q3 is bromine.

### Table 54

The compounds in Table 54 are of the general formula (I) where Q1 is chlorine, Q2, Q4, Q5 & Q6 are hydrogen, Q3 is bromine, m is 0, L is O, R¹ is ethyl, and R² and R³ have the values given in the table. Thus, compound 1 of Table 54 is the same as compound 1 of Table 1 except that in compound 1 of Table 54, Q1 is chlorine, Q3 is bromine, R¹ is ethyl. Similarly, compounds 2 to 299 of Table 54 are the same as compounds 2 to 299 of Table 1, respectively, except that in the compounds of Table 54, Q1 is chlorine, Q3 is bromine, R¹ is ethyl.

### Table 55

The compounds in Table 55 are of the general formula (I) where Q1 is chlorine, Q2, Q4, Q5 & Q6 are hydrogen, Q3 is bromine, m is 1, L is O, R¹ is methyl, and R² and R³ have the values given in the table. Thus, compound 1 of Table 55 is the same as compound 1 of Table 1 except that in compound 1 of Table 55, Q1 is chlorine, Q3 is bromine, m is 1. Similarly, compounds 2 to 299 of Table 55 are the same as compounds 2 to 299 of Table 1, respectively, except that in the compounds of Table 55, Q1 is chlorine, Q3 is bromine, m is 1.

### Table 56

The compounds in Table 56 are of the general formula (I) where Q1 is chlorine, Q2, Q4, Q5 & Q6 are hydrogen, Q3 is bromine, m is 1, L is O, R¹ is ethyl, and R² and R³ have the values given in the table. Thus, compound 1 of Table 56 is the same as compound 1 of Table 1 except that in compound 1 of Table 56, Q1 is chlorine, Q3 is bromine, m is 1, R¹ is ethyl. Similarly, compounds 2 to 299 of Table 56 are the same as compounds 2 to 299 of Table 1, respectively, except that in the compounds of Table 56, Q1 is chlorine, Q3 is bromine, m is 1, R¹ is ethyl.

### Table 57

The compounds in Table 57 are of the general formula (I) where Q1 is chlorine, Q2, Q4, Q5 & Q6 are hydrogen, Q3 is methyl, m is 0, L is O, R¹ is methyl, and R² and R³ have the values given in the table. Thus, compound 1 of Table 57 is the same as compound 1 of Table 1 except that in compound 1 of Table 57, Q1 is chlorine, Q3 is methyl. Similarly, compounds 2 to 299 of Table 57 are the same as compounds 2 to 299 of Table 1, respectively, except that in the compounds of Table 57, Q1 is chlorine, Q3 is methyl.

### Table 58

The compounds in Table 58 are of the general formula (I) where Q1 is chlorine, Q2, Q4, Q5 & Q6 are hydrogen, Q3 is methyl, m is 0, L is O, R¹ is ethyl, and R² and R³ have the values given in the table. Thus, compound 1 of Table 58 is the same as compound 1 of Table 1 except that in compound 1 of Table 58, Q1 is chlorine, Q3 is methyl, R¹ is ethyl. Similarly, compounds 2 to 299 of Table 58 are the same as compounds 2 to 299 of Table 1, respectively, except that in the compounds of Table 58, Q1 is chlorine, Q3 is methyl, R¹ is ethyl.

### Table 59

The compounds in Table 59 are of the general formula (I) where Q1 is chlorine, Q2, Q4, Q5 & Q6 are hydrogen, Q3 is methyl, m is 1, L is O, R¹ is methyl, and R² and R³ have the values given in the table. Thus, compound 1 of Table 59 is the same as compound 1 of Table 1 except that in compound 1 of Table 59, Q1 is chlorine, Q3 is methyl, m is 1. Similarly, compounds 2 to 299 of Table 59 are the same as compounds 2 to 299 of Table 1, respectively, except that in the compounds of Table 59, Q1 is chlorine, Q3 is methyl, m is 1.

### Table 60

The compounds in Table 60 are of the general formula (I) where Q1 is chlorine, Q2, Q4, Q5 & Q6 are hydrogen, Q3 is methyl, m is 1, L is O, R¹ is ethyl, and R² and R³ have the values given in the table. Thus, compound 1 of Table 60 is the same as compound 1 of Table 1 except that in compound 1 of Table 60, Q1 is chlorine, Q3 is methyl, m is 1, R¹ is ethyl. Similarly, compounds 2 to 299 of Table 60 are the same as compounds 2 to 299 of Table 1, respectively, except that in the compounds of Table 60, Q1 is chlorine, Q3 is methyl, m is 1, R¹ is ethyl.

### Table 61

The compounds in Table 61 are of the general formula (I) where Q1 is fluorine, Q3, Q4, Q5 & Q6 are hydrogen, Q2 is methyl, m is 0, L is O, R¹ is methyl, and R² and R³ have the values given in the table. Thus, compound 1 of Table 61 is the same as compound 1 of Table 1 except that in compound 1 of Table 61, Q1 is fluorine, Q2 is methyl. Similarly, compounds 2 to 299 of Table 61 are the same as compounds 2 to 299 of Table 1, respectively, except that in the compounds of Table 61, Q1 is fluorine, Q2 is methyl.

### Table 62

The compounds in Table 62 are of the general formula (I) where Q1 is fluorine, Q3, Q4, Q5 & Q6 are hydrogen, Q2 is methyl, m is 0, L is O, R¹ is ethyl, and R² and R³ have the values given in the table. Thus, compound 1 of Table 62 is the same as compound 1 of Table 1 except that in compound 1 of Table 62, Q1 is fluorine, Q2 is methyl, R¹ is ethyl. Similarly, compounds 2 to 299 of Table 62 are the same as compounds 2 to 299 of Table 1, respectively, except that in the compounds of Table 62, Q1 is fluorine, Q2 is methyl, R¹ is ethyl.

### Table 63

The compounds in Table 63 are of the general formula (I) where Q1 is fluorine, Q3, Q4, Q5 & Q6 are hydrogen, Q2 is methyl, m is 1, L is O, R¹ is methyl, and R² and R³ have the values given in the table. Thus, compound 1 of Table 63 is the same as compound 1 of Table 1 except that in compound 1 of Table 63, Q1 is fluorine, Q2 is methyl, m is 1. Similarly, compounds 2 to 299 of Table 63 are the same as compounds 2 to 299 of Table 1, respectively, except that in the compounds of Table 63, Q1 is fluorine, Q2 is methyl, m is 1.

### Table 64

The compounds in Table 64 are of the general formula (I) where Q1 is fluorine, Q3, Q4, Q5 & Q6 are hydrogen, Q2 is methyl, m is 1, L is O, R¹ is ethyl, and R² and R³ have the values given in the table. Thus, compound 1 of Table 64 is the same as compound 1 of Table 1 except that in compound 1 of Table 64, Q1 is fluorine, Q2 is methyl, m is 1, R¹ is ethyl. Similarly, compounds 2 to 299 of Table 64 are the same as compounds 2 to 299 of Table 1, respectively, except that in the compounds of Table 64, Q1 is fluorine, Q2 is methyl, m is 1, R¹ is ethyl.

### Table 65

The compounds in Table 65 are of the general formula (I) where Q1 is fluorine, Q3, Q4, Q5 & Q6 are hydrogen, Q2 is chlorine, m is 0, L is O, R¹ is methyl, and R² and R³ have the values given in the table. Thus, compound 1 of Table 65 is the same as compound 1 of Table 1 except that in compound 1 of Table 65, Q1 is fluorine, Q2 is chlorine. Similarly, compounds 2 to 299 of Table 65 are the same as compounds 2 to 299 of Table 1, respectively, except that in the compounds of Table 65, Q1 is fluorine, Q2 is chlorine.

### Table 66

The compounds in Table 66 are of the general formula (I) where Q1 is fluorine, Q3, Q4, Q5 & Q6 are hydrogen, Q2 is chlorine, m is 0, L is O, R¹ is ethyl, and R² and R³ have the values given in the table. Thus, compound 1 of Table 66 is the same as compound 1 of Table 1 except that in compound 1 of Table 66, Q1 is fluorine, Q2 is chlorine, R¹ is ethyl. Similarly, compounds 2 to 299 of Table 66 are the same as compounds 2 to 299 of Table 1, respectively, except that in the compounds of Table 66, Q1 is fluorine, Q2 is chlorine, R¹ is ethyl.

### Table 67

The compounds in Table 67 are of the general formula (I) where Q1 is fluorine, Q3, Q4, Q5 & Q6 are hydrogen, Q2 is chlorine, m is 1, L is O, R¹ is methyl, and R² and R³ have the values given in the table. Thus, compound 1 of Table 67 is the same as compound 1 of Table 1 except that in compound 1 of Table 67, Q1 is fluorine, Q2 is chlorine, m is 1. Similarly, compounds 2 to 299 of Table 67 are the same as compounds 2 to 299 of Table 1, respectively, except that in the compounds of Table 67, Q1 is fluorine, Q2 is chlorine, m is 1.

### Table 68

The compounds in Table 68 are of the general formula (I) where Q1 is fluorine, Q3, Q4, Q5 & Q6 are hydrogen, Q2 is chlorine, m is 1, L is O, R¹ is ethyl, and R² and R³ have the values given in the table. Thus, compound 1 of Table 68 is the same as compound 1 of Table 1 except that in compound 1 of Table 68, Q1 is fluorine, Q2 is chlorine, m is 1, R¹ is ethyl. Similarly, compounds 2 to 299 of Table 68 are the same as compounds 2 to 299 of Table 1, respectively, except that in the compounds of Table 68, Q1 is fluorine, Q2 is chlorine, m is 1, R¹ is ethyl.

### Table 69

The compounds in Table 69 are of the general formula (I) where Q1 is fluorine, Q3, Q4, Q5 & Q6 are hydrogen, Q2 is bromine, m is 0, L is O, R¹ is methyl, and R² and R³ have the values given in the table. Thus, compound 1 of Table 69 is the same as compound 1 of Table 1 except that in compound 1 of Table 69, Q1 is fluorine, Q2 is bromine. Similarly, compounds 2 to 299 of Table 69 are the same as compounds 2 to 299 of Table 1, respectively, except that in the compounds of Table 69, Q1 is fluorine, Q2 is bromine.

### Table 70

The compounds in Table 70 are of the general formula (I) where Q1 is fluorine, Q3, Q4, Q5 & Q6 are hydrogen, Q2 is bromine, m is 0, L is O, R¹ is ethyl, and R² and R³ have the values given in the table. Thus, compound 1 of Table 70 is the same as compound 1 of Table 1 except that in compound 1 of Table 70, Q1 is fluorine, Q2 is bromine, R¹ is ethyl. Similarly, compounds 2 to 299 of Table 70 are the same as compounds 2 to 299 of Table 1, respectively, except that in the compounds of Table 70, Q1 is fluorine, Q2 is bromine, R¹ is ethyl.

### Table 71

The compounds in Table 71 are of the general formula (I) where Q1 is fluorine, Q3, Q4, Q5 & Q6 are hydrogen, Q2 is bromine, m is 1, L is O, R¹ is methyl, and R² and R³ have the values given in the table. Thus, compound 1 of Table 71 is the same as compound 1 of Table 1 except that in compound 1 of Table 71, Q1 is fluorine, Q2 is bromine, m is 1. Similarly, compounds 2 to 299 of Table 71 are the same as compounds 2 to 299 of Table 1, respectively, except that in the compounds of Table 71, Q1 is fluorine, Q2 is bromine, m is 1.

### Table 72

The compounds in Table 72 are of the general formula (I) where Q1 is fluorine, Q3, Q4, Q5 & Q6 are hydrogen, Q2 is bromine, m is 1, L is O, R¹ is ethyl, and R² and R³ have the values given in the table. Thus, compound 1 of Table 72 is the same as compound 1 of Table 1 except that in compound 1 of Table 72, Q1 is fluorine, Q2 is bromine, m is 1, R¹ is ethyl. Similarly, compounds 2 to 299 of Table 72 are the same as compounds 2 to 299 of Table 1, respectively, except that in the compounds of Table 72, Q1 is fluorine, Q2 is bromine, m is 1, R¹ is ethyl.

### Table 73

The compounds in Table 73 are of the general formula (I) where Q1 is fluorine, Q2, Q4, Q5 & Q6 are hydrogen, Q3 is fluoro, m is 0, L is O, R¹ is methyl, and R² and R³ have the values given in the table. Thus, compound 1 of Table 73 is the same as compound 1 of Table 1 except that in compound 1 of Table 73, Q1 is fluorine, Q3 is fluoro. Similarly, compounds 2 to 299 of Table 73 are the same as compounds 2 to 299 of Table 1, respectively, except that in the compounds of Table 73, Q1 is fluorine, Q3 is fluoro.

### Table 74

The compounds in Table 74 are of the general formula (I) where Q1 is fluorine, Q2, Q4, Q5 & Q6 are hydrogen, Q3 is fluoro, m is 0, L is O, R¹ is ethyl, and R² and R³ have the values given in the table. Thus, compound 1 of Table 74 is the same as compound 1 of Table 1 except that in compound 1 of Table 74, Q1 is fluorine, Q3 is fluoro, R¹ is ethyl. Similarly, compounds 2 to 299 of Table 74 are the same as compounds 2 to 299 of Table 1, respectively, except that in the compounds of Table 74, Q1 is fluorine, Q3 is fluoro, R¹ is ethyl.

### Table 75

The compounds in Table 75 are of the general formula (I) where Q1 is fluorine, Q2, Q4, Q5 & Q6 are hydrogen, Q3 is fluoro, m is 1, L is O, R¹ is methyl, and R² and R³ have the values given in the table. Thus, compound 1 of Table 75 is the same as compound 1 of Table 1 except that in compound 1 of Table 75, Q1 is fluorine, Q3 is fluoro, m is 1. Similarly, compounds 2 to 299 of Table 75 are the same as compounds 2 to 299 of Table 1, respectively, except that in the compounds of Table 75, Q1 is fluorine, Q3 is fluoro, m is 1.

### Table 76

The compounds in Table 76 are of the general formula (I) where Q1 is fluorine, Q2, Q4, Q5 & Q6 are hydrogen, Q3 is fluoro, m is 1, L is O, R¹ is ethyl, and R² and R³ have the values given in the table. Thus, compound 1 of Table 76 is the same as compound 1 of Table 1 except that in compound 1 of Table 76, Q1 is fluorine, Q3 is fluoro, m is 1, R¹ is ethyl. Similarly, compounds 2 to 299 of Table 76 are the same as compounds 2 to 299 of Table 1, respectively, except that in the compounds of Table 76, Q1 is fluorine, Q3 is fluoro, m is 1, R¹ is ethyl.

### Table 77

The compounds in Table 77 are of the general formula (I) where Q1 is fluorine, Q2, Q4, Q5 & Q6 are hydrogen, Q3 is chlorine, m is 0, L is O, R¹ is methyl, and R² and R³ have the values given in the table. Thus, compound 1 of Table 77 is the same as compound 1 of Table 1 except that in compound 1 of Table 77, Q1 is fluorine, Q3 is chlorine. Similarly, compounds 2 to 299 of Table 77 are the same as compounds 2 to 299 of Table 1, respectively, except that in the compounds of Table 77, Q1 is fluorine, Q3 is chlorine.

### Table 78

The compounds in Table 78 are of the general formula (I) where Q1 is fluorine, Q2, Q4, Q5 & Q6 are hydrogen, Q3 is chlorine, m is 0, L is O, R¹ is ethyl, and R² and R³ have the values given in the table. Thus, compound 1 of Table 78 is the same as compound 1 of Table 1 except that in compound 1 of Table 78, Q1 is fluorine, Q3 is chlorine, R¹ is ethyl. Similarly, compounds 2 to 299 of Table 78 are the same as compounds 2 to 299 of Table 1, respectively, except that in the compounds of Table 78, Q1 is fluorine, Q3 is chlorine, R¹ is ethyl.

### Table 79

The compounds in Table 79 are of the general formula (I) where Q1 is fluorine, Q2, Q4, Q5 & Q6 are hydrogen, Q3 is chlorine, m is 1, L is O, R¹ is methyl, and R² and R³ have the values given in the table. Thus, compound 1 of Table 79 is the same as compound 1 of Table 1 except that in compound 1 of Table 79, Q1 is fluorine, Q3 is chlorine, m is 1. Similarly, compounds 2 to 299 of Table 79 are the same as compounds 2 to 299 of Table 1, respectively, except that in the compounds of Table 79, Q1 is fluorine, Q3 is chlorine, m is 1.

### Table 80

The compounds in Table 80 are of the general formula (I) where Q1 is fluorine, Q2, Q4, Q5 & Q6 are hydrogen, Q3 is chlorine, m is 1, L is O, R¹ is ethyl, and R² and R³ have the values given in the table. Thus, compound 1 of Table 80 is the same as compound 1 of Table 1 except that in compound 1 of Table 80, Q1 is fluorine, Q3 is chlorine, m is 1, R¹ is ethyl. Similarly, compounds 2 to 299 of Table 80 are the same as compounds 2 to 299 of Table 1, respectively, except that in the compounds of Table 80, Q1 is fluorine, Q3 is chlorine, m is 1, R¹ is ethyl.

### Table 81

The compounds in Table 81 are of the general formula (I) where Q1 is fluorine, Q2, Q4, Q5 & Q6 are hydrogen, Q3 is bromine, m is 0, L is O, R¹ is methyl, and R² and R³ have the values given in the table. Thus, compound 1 of Table 81 is the same as compound 1 of Table 1 except that in compound 1 of Table 81, Q1 is fluorine, Q3 is bromine. Similarly, compounds 2 to 299 of Table 81 are the same as compounds 2 to 299 of Table 1, respectively, except that in the compounds of Table 81, Q1 is fluorine, Q3 is bromine.

### Table 82

The compounds in Table 82 are of the general formula (I) where Q1 is fluorine, Q2, Q4, Q5 & Q6 are hydrogen, Q3 is bromine, m is 0, L is O, R¹ is ethyl, and R² and R³ have the values given in the table. Thus, compound 1 of Table 82 is the same as compound 1 of Table 1 except that in compound 1 of Table 82, Q1 is fluorine, Q3 is bromine, R¹ is ethyl. Similarly, compounds 2 to 299 of Table 82 are the same as compounds 2 to 299 of Table 1, respectively, except that in the compounds of Table 82, Q1 is fluorine, Q3 is bromine, R¹ is ethyl.

### Table 83

The compounds in Table 83 are of the general formula (I) where Q1 is fluorine, Q2, Q4, Q5 & Q6 are hydrogen, Q3 is bromine, m is 1, L is O, R¹ is methyl, and R² and R³ have the values given in the table. Thus, compound 1 of Table 83 is the same as compound 1 of Table 1 except that in compound 1 of Table 83, Q1 is fluorine, Q3 is bromine, m is 1. Similarly, compounds 2 to 299 of Table 83 are the same as compounds 2 to 299 of Table 1, respectively, except that in the compounds of Table 83, Q1 is fluorine, Q3 is bromine, m is 1.

### Table 84

The compounds in Table 84 are of the general formula (I) where Q1 is fluorine, Q2, Q4, Q5 & Q6 are hydrogen, Q3 is bromine, m is 1, L is O, R¹ is ethyl, and R² and R³ have the values given in the table. Thus, compound 1 of Table 84 is the same as compound 1 of Table 1 except that in compound 1 of Table 84, Q1 is fluorine, Q3 is bromine, m is 1, R¹ is ethyl. Similarly, compounds 2 to 299 of Table 84 are the same as compounds 2 to 299 of Table 1, respectively, except that in the compounds of Table 84, Q1 is fluorine, Q3 is bromine, m is 1, R¹ is ethyl.

### Table 85

The compounds in Table 85 are of the general formula (I) where Q1 is fluorine, Q2, Q4, Q5 & Q6 are hydrogen, Q3 is methyl, m is 0, L is O, R¹ is methyl, and R² and R³ have the values given in the table. Thus, compound 1 of Table 85 is the same as compound 1 of Table 1 except that in compound 1 of Table 85, Q1 is fluorine, Q3 is methyl. Similarly, compounds 2 to 299 of Table 85 are the same as compounds 2 to 299 of Table 1, respectively, except that in the compounds of Table 85, Q1 is fluorine, Q3 is methyl.

### Table 86

The compounds in Table 86 are of the general formula (I) where Q1 is fluorine, Q2, Q4, Q5 & Q6 are hydrogen, Q3 is methyl, m is 0, L is O, R¹ is ethyl, and R² and R³ have the values given in the table. Thus, compound 1 of Table 86 is the same as compound 1 of Table 1 except that in compound 1 of Table 86, Q1 is fluorine, Q3 is methyl, R¹ is ethyl. Similarly, compounds 2 to 299 of Table 86 are the same as compounds 2 to 299 of Table 1, respectively, except that in the compounds of Table 86, Q1 is fluorine, Q3 is methyl, R¹ is ethyl.

### Table 87

The compounds in Table 87 are of the general formula (I) where Q1 is fluorine, Q2, Q4, Q5 & Q6 are hydrogen, Q3 is methyl, m is 1, L is O, R¹ is methyl, and R² and R³ have the values given in the table. Thus, compound 1 of Table 87 is the same as compound 1 of Table 1 except that in compound 1 of Table 87, Q1 is fluorine, Q3 is methyl, m is 1. Similarly, compounds 2 to 299 of Table 87 are the same as compounds 2 to 299 of Table 1, respectively, except that in the compounds of Table 87, Q1 is fluorine, Q3 is methyl, m is 1.

### Table 88

The compounds in Table 88 are of the general formula (I) where Q1 is fluorine, Q2, Q4, Q5 & Q6 are hydrogen, Q3 is methyl, m is 1, L is O, R¹ is ethyl, and R² and R³ have the values given in the table. Thus, compound 1 of Table 88 is the same as compound 1 of Table 1 except that in compound 1 of Table 88, Q1 is fluorine, Q3 is methyl, m is 1, R¹ is ethyl. Similarly, compounds 2 to 299 of Table 88 are the same as compounds 2 to 299 of Table 1, respectively, except that in the compounds of Table 88, Q1 is fluorine, Q3 is methyl, m is 1, R¹ is ethyl.

### Table 89

The compounds in Table 89 are of the general formula (I) where Q1 is bromine, Q3, Q4, Q5 & Q6 are hydrogen, Q2 is methyl, m is 0, L is O, R¹ is methyl, and R² and R³ have the values given in the table. Thus, compound 1 of Table 89 is the same as compound 1 of Table 1 except that in compound 1 of Table 89, Q1 is bromine, Q2 is methyl. Similarly, compounds 2 to 299 of Table 89 are the same as compounds 2 to 299 of Table 1, respectively, except that in the compounds of Table 89, Q1 is bromine, Q2 is methyl.

### Table 90

The compounds in Table 90 are of the general formula (I) where Q1 is bromine, Q3, Q4, Q5 & Q6 are hydrogen, Q2 is methyl, m is 0, L is O, R¹ is ethyl, and R² and R³ have the values given in the table. Thus, compound 1 of Table 90 is the same as compound 1 of Table 1 except that in compound 1 of Table 90, Q1 is bromine, Q2 is methyl, R¹ is ethyl. Similarly, compounds 2 to 299 of Table 90 are the same as compounds 2 to 299 of Table 1, respectively, except that in the compounds of Table 90, Q1 is bromine, Q2 is methyl, R¹ is ethyl.

### Table 91

The compounds in Table 91 are of the general formula (I) where Q1 is bromine, Q3, Q4, Q5 & Q6 are hydrogen, Q2 is methyl, m is 1, L is O, R¹ is methyl, and R² and R³ have the values given in the table. Thus, compound 1 of Table 91 is the same as compound 1 of Table 1 except that in compound 1 of Table 91, Q1 is bromine, Q2 is methyl, m is 1. Similarly, compounds 2 to 299 of Table 91 are the same as compounds 2 to 299 of Table 1, respectively, except that in the compounds of Table 91, Q1 is bromine, Q2 is methyl, m is 1.

### Table 92

The compounds in Table 92 are of the general formula (I) where Q1 is bromine, Q3, Q4, Q5 & Q6 are hydrogen, Q2 is methyl, m is 1, L is O, R¹ is ethyl, and R² and R³ have the values given in the table. Thus, compound 1 of Table 92 is the same as compound 1 of Table 1 except that in compound 1 of Table 92, Q1 is bromine, Q2 is methyl, m is 1, R¹ is ethyl. Similarly, compounds 2 to 299 of Table 92 are the same as compounds 2 to 299 of Table 1, respectively, except that in the compounds of Table 92, Q1 is bromine, Q2 is methyl, m is 1, R¹ is ethyl.

### Table 93

The compounds in Table 93 are of the general formula (I) where Q1 is bromine, Q3, Q4, Q5 & Q6 are hydrogen, Q2 is chlorine, m is 0, L is O, R¹ is methyl, and R² and R³ have the values given in the table. Thus, compound 1 of Table 93 is the same as compound 1 of Table 1 except that in compound 1 of Table 93, Q1 is bromine, Q2 is chlorine. Similarly, compounds 2 to 299 of Table 93 are the same as compounds 2 to 299 of Table 1, respectively, except that in the compounds of Table 93, Q1 is bromine, Q2 is chlorine.

### Table 94

The compounds in Table 94 are of the general formula (I) where Q1 is bromine, Q3, Q4, Q5 & Q6 are hydrogen, Q2 is chlorine, m is 0, L is O, R¹ is ethyl, and R² and R³ have the values given in the table. Thus, compound 1 of Table 94 is the same as compound 1 of Table 1 except that in compound 1 of Table 94, Q1 is bromine, Q2 is chlorine, R¹ is ethyl. Similarly, compounds 2 to 299 of Table 94 are the same as compounds 2 to 299 of Table 1, respectively, except that in the compounds of Table 94, Q1 is bromine, Q2 is chlorine, R¹ is ethyl.

### Table 95

The compounds in Table 95 are of the general formula (I) where Q1 is bromine, Q3, Q4, Q5 & Q6 are hydrogen, Q2 is chlorine, m is 1, L is O, R¹ is methyl, and R² and R³ have the values given in the table. Thus, compound 1 of Table 95 is the same as compound 1 of Table 1 except that in compound 1 of Table 95, Q1 is bromine, Q2 is chlorine, m is 1. Similarly, compounds 2 to 299 of Table 95 are the same as compounds 2 to 299 of Table 1, respectively, except that in the compounds of Table 95, Q1 is bromine, Q2 is chlorine, m is 1.

### Table 96

The compounds in Table 96 are of the general formula (I) where Q1 is bromine, Q3, Q4, Q5 & Q6 are hydrogen, Q2 is chlorine, m is 1, L is O, R¹ is ethyl, and R² and R³ have the values given in the table. Thus, compound 1 of Table 96 is the same as compound 1 of Table 1 except that in compound 1 of Table 96, Q1 is bromine, Q2 is chlorine, m is 1, R¹ is ethyl. Similarly, compounds 2 to 299 of Table 96 are the same as compounds 2 to 299 of Table 1, respectively, except that in the compounds of Table 96, Q1 is bromine, Q2 is chlorine, m is 1, R¹ is ethyl.

### Table 97

The compounds in Table 97 are of the general formula (I) where Q1 is bromine, Q3, Q4, Q5 & Q6 are hydrogen, Q2 is bromine, m is 0, L is O, R¹ is methyl, and R² and R³ have the values given in the table. Thus, compound 1 of Table 97 is the same as compound 1 of Table 1 except that in compound 1 of Table 97, Q1 is bromine, Q2 is bromine. Similarly, compounds 2 to 299 of Table 97 are the same as compounds 2 to 299 of Table 1, respectively, except that in the compounds of Table 97, Q1 is bromine, Q2 is bromine.

### Table 98

The compounds in Table 98 are of the general formula (I) where Q1 is bromine, Q3, Q4, Q5 & Q6 are hydrogen, Q2 is bromine, m is 0, L is O, R¹ is ethyl, and R² and R³ have the values given in the table. Thus, compound 1 of Table 98 is the same as compound 1 of Table 1 except that in compound 1 of Table 98, Q1 is bromine, Q2 is bromine, R¹ is ethyl. Similarly, compounds 2 to 299 of Table 98 are the same as compounds 2 to 299 of Table 1, respectively, except that in the compounds of Table 98, Q1 is bromine, Q2 is bromine, R¹ is ethyl.

### Table 99

The compounds in Table 99 are of the general formula (I) where Q1 is bromine, Q3, Q4, Q5 & Q6 are hydrogen, Q2 is bromine, m is 1, L is O, R¹ is methyl, and R² and R³ have the values given in the table. Thus, compound 1 of Table 99 is the same as compound 1 of Table 1 except that in compound 1 of Table 99, Q1 is bromine, Q2 is bromine, m is 1. Similarly, compounds 2 to 299 of Table 99 are the same as compounds 2 to 299 of Table 1, respectively, except that in the compounds of Table 99, Q1 is bromine, Q2 is bromine, m is 1.

### Table 100

The compounds in Table 100 are of the general formula (I) where Q1 is bromine, Q3, Q4, Q5 & Q6 are hydrogen, Q2 is bromine, m is 1, L is O, R¹ is ethyl, and R² and R³ have the values given in the table. Thus, compound 1 of Table 100 is the same as compound 1 of Table 1 except that in compound 1 of Table 100, Q1 is bromine, Q2 is bromine, m is 1, R¹ is ethyl. Similarly, compounds 2 to 299 of Table 100 are the same as compounds 2 to 299 of Table 1, respectively, except that in the compounds of Table 100, Q1 is bromine, Q2 is bromine, m is 1, R¹ is ethyl.

### Table 101

The compounds in Table 101 are of the general formula (I) where Q1 is bromine, Q2, Q4, Q5 & Q6 are hydrogen, Q3 is fluoro, m is 0, L is O, R¹ is methyl, and R² and R³ have the values given in the table. Thus, compound 1 of Table 101 is the same as compound 1 of Table 1 except that in compound 1 of Table 101, Q1 is bromine, Q3 is fluoro. Similarly, compounds 2 to 299 of Table 101 are the same as compounds 2 to 299 of Table 1, respectively, except that in the compounds of Table 101, Q1 is bromine, Q3 is fluoro.

### Table 102

The compounds in Table 102 are of the general formula (I) where Q1 is bromine, Q2, Q4, Q5 & Q6 are hydrogen, Q3 is fluoro, m is 0, L is O, R¹ is ethyl, and R² and R³ have the values given in the table. Thus, compound 1 of Table 102 is the same as compound 1 of Table 1 except that in compound 1 of Table 102, Q1 is bromine, Q3 is fluoro, R¹ is ethyl. Similarly, compounds 2 to 299 of Table 102 are the same as compounds 2 to 299 of Table 1, respectively, except that in the compounds of Table 102, Q1 is bromine, Q3 is fluoro, R¹ is ethyl.

### Table 103

The compounds in Table 103 are of the general formula (I) where Q1 is bromine, Q2, Q4, Q5 & Q6 are hydrogen, Q3 is fluoro, m is 1, L is O, R¹ is methyl, and R² and R³ have the values given in the table. Thus, compound 1 of Table 103 is the same as compound 1 of Table 1 except that in compound 1 of Table 103, Q1 is bromine, Q3 is fluoro, m is 1. Similarly, compounds 2 to 299 of Table 103 are the same as compounds 2 to 299 of Table 1, respectively, except that in the compounds of Table 103, Q1 is bromine, Q3 is fluoro, m is 1.

### Table 104

The compounds in Table 104 are of the general formula (I) where Q1 is bromine, Q2, Q4, Q5 & Q6 are hydrogen, Q3 is fluoro, m is 1, L is O, R¹ is ethyl, and R² and R³ have the values given in the table. Thus, compound 1 of Table 104 is the same as compound 1 of Table 1 except that in compound 1 of Table 104, Q1 is bromine, Q3 is fluoro, m is 1, R¹ is ethyl. Similarly, compounds 2 to 299 of Table 104 are the same as compounds 2 to 299 of Table 1, respectively, except that in the compounds of Table 104, Q1 is bromine, Q3 is fluoro, m is 1, R¹ is ethyl.

### Table 105

The compounds in Table 105 are of the general formula (I) where Q1 is bromine, Q2, Q4, Q5 & Q6 are hydrogen, Q3 is chlorine, m is 0, L is O, R¹ is methyl, and R² and R³ have the values given in the table. Thus, compound 1 of Table 105 is the same as compound 1 of Table 1 except that in compound 1 of Table 105, Q1 is bromine, Q3 is chlorine. Similarly, compounds 2 to 299 of Table 105 are the same as compounds 2 to 299 of Table 1, respectively, except that in the compounds of Table 105, Q1 is bromine, Q3 is chlorine.

### Table 106

The compounds in Table 106 are of the general formula (I) where Q1 is bromine, Q2, Q4, Q5 & Q6 are hydrogen, Q3 is chlorine, m is 0, L is O, R¹ is ethyl, and R² and R³ have the values given in the table. Thus, compound 1 of Table 106 is the same as compound 1 of Table 1 except that in compound 1 of Table 106, Q1 is bromine, Q3 is chlorine, R¹ is ethyl. Similarly, compounds 2 to 299 of Table 106 are the same as compounds 2 to 299 of Table 1, respectively, except that in the compounds of Table 106, Q1 is bromine, Q3 is chlorine, R¹ is ethyl.

### Table 107

The compounds in Table 107 are of the general formula (I) where Q1 is bromine, Q2, Q4, Q5 & Q6 are hydrogen, Q3 is chlorine, m is 1, L is O, R¹ is methyl, and R² and R³ have the values given in the table. Thus, compound 1 of Table 107 is the same as compound 1 of Table 1 except that in compound 1 of Table 107, Q1 is bromine, Q3 is chlorine, m is 1. Similarly, compounds 2 to 299 of Table 107 are the same as compounds 2 to 299 of Table 1, respectively, except that in the compounds of Table 107, Q1 is bromine, Q3 is chlorine, m is 1.

### Table 108

The compounds in Table 108 are of the general formula (I) where Q1 is bromine, Q2, Q4, Q5 & Q6 are hydrogen, Q3 is chlorine, m is 1, L is O, R¹ is ethyl, and R² and R³ have the values given in the table. Thus, compound 1 of Table 108 is the same as compound 1 of Table 1 except that in compound 1 of Table 108, Q1 is bromine, Q3 is chlorine, m is 1, R¹ is ethyl. Similarly, compounds 2 to 299 of Table 108 are the same as compounds 2 to 299 of Table 1, respectively, except that in the compounds of Table 108, Q1 is bromine, Q3 is chlorine, m is 1, R¹ is ethyl.

### Table 109

The compounds in Table 109 are of the general formula (I) where Q1 is bromine, Q2, Q4, Q5 & Q6 are hydrogen, Q3 is bromine, m is 0, L is O, R¹ is methyl, and R² and R³ have the values given in the table. Thus, compound 1 of Table 109 is the same as compound 1 of Table 1 except that in compound 1 of Table 109, Q1 is bromine, Q3 is bromine. Similarly, compounds 2 to 299 of Table 109 are the same as compounds 2 to 299 of Table 1, respectively, except that in the compounds of Table 109, Q1 is bromine, Q3 is bromine.

### Table 110

The compounds in Table 110 are of the general formula (I) where Q1 is bromine, Q2, Q4, Q5 & Q6 are hydrogen, Q3 is bromine, m is 0, L is O, R¹ is ethyl, and R² and R³ have the values given in the table. Thus, compound 1 of Table 110 is the same as compound 1 of Table 1 except that in compound 1 of Table 110, Q1 is bromine, Q3 is bromine, R¹ is ethyl. Similarly, compounds 2 to 299 of Table 110 are the same as compounds 2 to 299 of Table 1, respectively, except that in the compounds of Table 110, Q1 is bromine, Q3 is bromine, R¹ is ethyl.

### Table 111

The compounds in Table 111 are of the general formula (I) where Q1 is bromine, Q2, Q4, Q5 & Q6 are hydrogen, Q3 is bromine, m is 1, L is O, R¹ is methyl, and R² and R³ have the values given in the table. Thus, compound 1 of Table 111 is the same as compound 1 of Table 1 except that in compound 1 of Table 111, Q1 is bromine, Q3 is bromine, m is 1. Similarly, compounds 2 to 299 of Table 111 are the same as compounds 2 to 299 of Table 1, respectively, except that in the compounds of Table 111, Q1 is bromine, Q3 is bromine, m is 1.

### Table 112

The compounds in Table 112 are of the general formula (I) where Q1 is bromine, Q2, Q4, Q5 & Q6 are hydrogen, Q3 is bromine, m is 1, L is O, R¹ is ethyl, and R² and R³ have the values given in the table. Thus, compound 1 of Table 112 is the same as compound 1 of Table 1 except that in compound 1 of Table 112, Q1 is bromine, Q3 is bromine, m is 1, R¹ is ethyl. Similarly, compounds 2 to 299 of Table 112 are the same as compounds 2 to 299 of Table 1, respectively, except that in the compounds of Table 112, Q1 is bromine, Q3 is bromine, m is 1, R¹ is ethyl.

### Table 113

The compounds in Table 113 are of the general formula (I) where Q1 is bromine, Q2, Q4, Q5 & Q6 are hydrogen, Q3 is methyl, m is 0, L is O, R¹ is methyl, and R² and R³ have the values given in the table. Thus, compound 1 of Table 113 is the same as compound 1 of Table 1 except that in compound 1 of Table 113, Q1 is bromine, Q3 is methyl. Similarly, compounds 2 to 299 of Table 113 are the same as compounds 2 to 299 of Table 1, respectively, except that in the compounds of Table 113, Q1 is bromine, Q3 is methyl.

### Table 114

The compounds in Table 114 are of the general formula (I) where Q1 is bromine, Q2, Q4, Q5 & Q6 are hydrogen, Q3 is methyl, m is 0, L is O, R¹ is ethyl, and R² and R³ have the values given in the table. Thus, compound 1 of Table 114 is the same as compound 1 of Table 1 except that in compound 1 of Table 114, Q1 is bromine, Q3 is methyl, R¹ is ethyl. Similarly, compounds 2 to 299 of Table 114 are the same as compounds 2 to 299 of Table 1, respectively, except that in the compounds of Table 114, Q1 is bromine, Q3 is methyl, R¹ is ethyl.

### Table 115

The compounds in Table 115 are of the general formula (I) where Q1 is bromine, Q2, Q4, Q5 & Q6 are hydrogen, Q3 is methyl, m is 1, L is O, R¹ is methyl, and R² and R³ have the values given in the table. Thus, compound 1 of Table 115 is the same as compound 1 of Table 1 except that in compound 1 of Table 115, Q1 is bromine, Q3 is methyl, m is 1. Similarly, compounds 2 to 299 of Table 115 are the same as compounds 2 to 299 of Table 1, respectively, except that in the compounds of Table 115, Q1 is bromine, Q3 is methyl, m is 1.

### Table 116

The compounds in Table 116 are of the general formula (I) where Q1 is bromine, Q2, Q4, Q5 & Q6 are hydrogen, Q3 is methyl, m is 1, L is O, R¹ is ethyl, and R² and R³ have the values given in the table. Thus, compound 1 of Table 116 is the same as compound 1 of Table 1 except that in compound 1 of Table 116, Q1 is bromine, Q3 is methyl, m is 1, R¹ is ethyl. Similarly, compounds 2 to 299 of Table 116 are the same as compounds 2 to 299 of Table 1, respectively, except that in the compounds of Table 116, Q1 is bromine, Q3 is methyl, m is 1, R¹ is ethyl.

### Table 117

The compounds in Table 117 are of the general formula (I) where Q1 is iodine, Q3, Q4, Q5 & Q6 are hydrogen, Q2 is methyl, m is 0, L is O, R¹ is methyl, and R² and R³ have the values given in the table. Thus, compound 1 of Table 117 is the same as compound 1 of Table 1 except that in compound 1 of Table 117, Q1 is iodine, Q2 is methyl. Similarly, compounds 2 to 299 of Table 117 are the same as compounds 2 to 299 of Table 1, respectively, except that in the compounds of Table 117, Q1 is iodine, Q2 is methyl.

### Table 118

The compounds in Table 118 are of the general formula (I) where Q1 is iodine, Q3, Q4, Q5 & Q6 are hydrogen, Q2 is methyl, m is 0, L is O, R¹ is ethyl, and R² and R³ have the values given in the table. Thus, compound 1 of Table 118 is the same as compound 1 of Table 1 except that in compound 1 of Table 118, Q1 is iodine, Q2 is methyl, R¹ is ethyl. Similarly, compounds 2 to 299 of Table 118 are the same as compounds 2 to 299 of Table 1, respectively, except that in the compounds of Table 118, Q1 is iodine, Q2 is methyl, R¹ is ethyl.

### Table 119

The compounds in Table 119 are of the general formula (I) where Q1 is iodine, Q3, Q4, Q5 & Q6 are hydrogen, Q2 is methyl, m is 1, L is O, R¹ is methyl, and R² and R³ have the values given in the table. Thus, compound 1 of Table 119 is the same as compound 1 of Table 1 except that in compound 1 of Table 119, Q1 is iodine, Q2 is methyl, m is 1. Similarly, compounds 2 to 299 of Table 119 are the same as compounds 2 to 299 of Table 1, respectively, except that in the compounds of Table 119, Q1 is iodine, Q2 is methyl, m is 1.

### Table 120

The compounds in Table 120 are of the general formula (I) where Q1 is iodine, Q3, Q4, Q5 & Q6 are hydrogen, Q2 is methyl, m is 1, L is O, R¹ is ethyl, and R² and R³ have the values given in the table. Thus, compound 1 of Table 120 is the same as compound 1 of Table 1 except that in compound 1 of Table 120, Q1 is iodine, Q2 is methyl, m is 1, R¹ is ethyl. Similarly, compounds 2 to 299 of Table 120 are the same as compounds 2 to 299 of Table 1, respectively, except that in the compounds of Table 120, Q1 is iodine, Q2 is methyl, m is 1, R¹ is ethyl.

### Table 121

The compounds in Table 121 are of the general formula (I) where Q1 is iodine, Q3, Q4, Q5 & Q6 are hydrogen, Q2 is chlorine, m is 0, L is O, R¹ is methyl, and R² and R³ have the values given in the table. Thus, compound 1 of Table 121 is the same as compound 1 of Table 1 except that in compound 1 of Table 121, Q1 is iodine, Q2 is chlorine. Similarly, compounds 2 to 299 of Table 121 are the same as compounds 2 to 299 of Table 1, respectively, except that in the compounds of Table 121, Q1 is iodine, Q2 is chlorine.

### Table 122

The compounds in Table 122 are of the general formula (I) where Q1 is iodine, Q3, Q4, Q5 & Q6 are hydrogen, Q2 is chlorine, m is 0, L is O, R¹ is ethyl, and R² and R³ have the values given in the table. Thus, compound 1 of Table 122 is the same as compound 1 of Table 1 except that in compound 1 of Table 122, Q1 is iodine, Q2 is chlorine, R¹ is ethyl. Similarly, compounds 2 to 299 of Table 122 are the same as compounds 2 to 299 of Table 1, respectively, except that in the compounds of Table 122, Q1 is iodine, Q2 is chlorine, R¹ is ethyl.

### Table 123

The compounds in Table 123 are of the general formula (I) where Q1 is iodine, Q3, Q4, Q5 & Q6 are hydrogen, Q2 is chlorine, m is 1, L is O, R¹ is methyl, and R² and R³ have the values given in the table. Thus, compound 1 of Table 123 is the same as compound 1 of Table 1 except that in compound 1 of Table 123, Q1 is iodine, Q2 is chlorine, m is 1. Similarly, compounds 2 to 299 of Table 123 are the same as compounds 2 to 299 of Table 1, respectively, except that in the compounds of Table 123, Q1 is iodine, Q2 is chlorine, m is 1.

### Table 124

The compounds in Table 124 are of the general formula (I) where Q1 is iodine, Q3, Q4, Q5 & Q6 are hydrogen, Q2 is chlorine, m is 1, L is O, R¹ is ethyl, and R² and R³ have the values given in the table. Thus, compound 1 of Table 124 is the same as compound 1 of Table 1 except that in compound 1 of Table 124, Q1 is iodine, Q2 is chlorine, m is 1, R¹ is ethyl. Similarly, compounds 2 to 299 of Table 124 are the same as compounds 2 to 299 of Table 1, respectively, except that in the compounds of Table 124, Q1 is iodine, Q2 is chlorine, m is 1, R¹ is ethyl.

### Table 125

The compounds in Table 125 are of the general formula (I) where Q1 is iodine, Q3, Q4, Q5 & Q6 are hydrogen, Q2 is bromine, m is 0, L is O, R¹ is methyl, and R² and R³ have the values given in the table. Thus, compound 1 of Table 125 is the same as compound 1 of Table 1 except that in compound 1 of Table 125, Q1 is iodine, Q2 is bromine. Similarly, compounds 2 to 299 of Table 125 are the same as compounds 2 to 299 of Table 1, respectively, except that in the compounds of Table 125, Q1 is iodine, Q2 is bromine.

### Table 126

The compounds in Table 126 are of the general formula (I) where Q1 is iodine, Q3, Q4, Q5 & Q6 are hydrogen, Q2 is bromine, m is 0, L is O, R¹ is ethyl, and R² and R³ have the values given in the table. Thus, compound 1 of Table 126 is the same as compound 1 of Table 1 except that in compound 1 of Table 126, Q1 is iodine, Q2 is bromine, R¹ is ethyl. Similarly, compounds 2 to 299 of Table 126 are the same as compounds 2 to 299 of Table 1, respectively, except that in the compounds of Table 126, Q1 is iodine, Q2 is bromine, R¹ is ethyl.

### Table 127

The compounds in Table 127 are of the general formula (I) where Q1 is iodine, Q3, Q4, Q5 & Q6 are hydrogen, Q2 is bromine, m is 1, L is O, R¹ is methyl, and R² and R³ have the values given in the table. Thus, compound 1 of Table 127 is the same as compound 1 of Table 1 except that in compound 1 of Table 127, Q1 is iodine, Q2 is bromine, m is 1. Similarly, compounds 2 to 299 of Table 127 are the same as compounds 2 to 299 of Table 1, respectively, except that in the compounds of Table 127, Q1 is iodine, Q2 is bromine, m is 1.

### Table 128

The compounds in Table 128 are of the general formula (I) where Q1 is iodine, Q3, Q4, Q5 & Q6 are hydrogen, Q2 is bromine, m is 1, L is O, R¹ is ethyl, and R² and R³ have the values given in the table. Thus, compound 1 of Table 128 is the same as compound 1 of Table 1 except that in compound 1 of Table 128, Q1 is iodine, Q2 is bromine, m is 1, R¹ is ethyl. Similarly, compounds 2 to 299 of Table 128 are the same as compounds 2 to 299 of Table 1, respectively, except that in the compounds of Table 128, Q1 is iodine, Q2 is bromine, m is 1, R¹ is ethyl.

### Table 129

The compounds in Table 129 are of the general formula (I) where Q1 is iodine, Q2, Q4, Q5 & Q6 are hydrogen, Q3 is fluoro, m is 0, L is O, R¹ is methyl, and R² and R³ have the values given in the table. Thus, compound 1 of Table 129 is the same as compound 1 of Table 1 except that in compound 1 of Table 129, Q1 is iodine, Q3 is fluoro. Similarly, compounds 2 to 299 of Table 129 are the same as compounds 2 to 299 of Table 1, respectively, except that in the compounds of Table 129, Q1 is iodine, Q3 is fluoro.

### Table 130

The compounds in Table 130 are of the general formula (I) where Q1 is iodine, Q2, Q4, Q5 & Q6 are hydrogen, Q3 is fluoro, m is 0, L is O, R¹ is ethyl, and R² and R³ have the values given in the table. Thus, compound 1 of Table 130 is the same as compound 1 of Table 1 except that in compound 1 of Table 130, Q1 is iodine, Q3 is fluoro, R¹ is ethyl. Similarly, compounds 2 to 299 of Table 130 are the same as compounds 2 to 299 of Table 1, respectively, except that in the compounds of Table 130, Q1 is iodine, Q3 is fluoro, R¹ is ethyl.

### Table 131

The compounds in Table 131 are of the general formula (I) where Q1 is iodine, Q2, Q4, Q5 & Q6 are hydrogen, Q3 is fluoro, m is 1, L is O, R¹ is methyl, and R² and R³ have the values given in the table. Thus, compound 1 of Table 131 is the same as compound 1 of Table 1 except that in compound 1 of Table 131, Q1 is iodine, Q3 is fluoro, m is 1. Similarly, compounds 2 to 299 of Table 131 are the same as compounds 2 to 299 of Table 1, respectively, except that in the compounds of Table 131, Q1 is iodine, Q3 is fluoro, m is 1.

### Table 132

The compounds in Table 132 are of the general formula (I) where Q1 is iodine, Q2, Q4, Q5 & Q6 are hydrogen, Q3 is fluoro, m is 1, L is O, R¹ is ethyl, and R² and R³ have the values given in the table. Thus, compound 1 of Table 132 is the same as compound 1 of Table 1 except that in compound 1 of Table 132, Q1 is iodine, Q3 is fluoro, m is 1, R¹ is ethyl. Similarly, compounds 2 to 299 of Table 132 are the same as compounds 2 to 299 of Table 1, respectively, except that in the compounds of Table 132, Q1 is iodine, Q3 is fluoro, m is 1, R¹ is ethyl.

### Table 133

The compounds in Table 133 are of the general formula (I) where Q1 is iodine, Q2, Q4, Q5 & Q6 are hydrogen, Q3 is chlorine, m is 0, L is O, R¹ is methyl, and R² and R³ have the values given in the table. Thus, compound 1 of Table 133 is the same as compound 1 of Table 1 except that in compound 1 of Table 133, Q1 is iodine, Q3 is chlorine. Similarly, compounds 2 to 299 of Table 133 are the same as compounds 2 to 299 of Table 1, respectively, except that in the compounds of Table 133, Q1 is iodine, Q3 is chlorine.

### Table 134

The compounds in Table 134 are of the general formula (I) where Q1 is iodine, Q2, Q4, Q5 & Q6 are hydrogen, Q3 is chlorine, m is 0, L is O, R¹ is ethyl, and R² and R³ have the values given in the table. Thus, compound 1 of Table 134 is the same as compound 1 of Table 1 except that in compound 1 of Table 134, Q1 is iodine, Q3 is chlorine, R¹ is ethyl. Similarly, compounds 2 to 299 of Table 134 are the same as compounds 2 to 299 of Table 1, respectively, except that in the compounds of Table 134, Q1 is iodine, Q3 is chlorine, R¹ is ethyl.

### Table 135

The compounds in Table 135 are of the general formula (I) where Q1 is iodine, Q2, Q4, Q5 & Q6 are hydrogen, Q3 is chlorine, m is 1, L is O, R¹ is methyl, and R² and R³ have the values given in the table. Thus, compound 1 of Table 135 is the same as compound 1 of Table 1 except that in compound 1 of Table 135, Q1 is iodine, Q3 is chlorine, m is 1. Similarly, compounds 2 to 299 of Table 135 are the same as compounds 2 to 299 of Table 1, respectively, except that in the compounds of Table 135, Q1 is iodine, Q3 is chlorine, m is 1.

### Table 136

The compounds in Table 136 are of the general formula (I) where Q1 is iodine, Q2, Q4, Q5 & Q6 are hydrogen, Q3 is chlorine, m is 1, L is O, R¹ is ethyl, and R² and R³ have the values given in the table. Thus, compound 1 of Table 136 is the same as compound 1 of Table 1 except that in compound 1 of Table 136, Q1 is iodine, Q3 is chlorine, m is 1, R¹ is ethyl. Similarly, compounds 2 to 299 of Table 136 are the same as compounds 2 to 299 of Table 1, respectively, except that in the compounds of Table 136, Q1 is iodine, Q3 is chlorine, m is 1, R¹ is ethyl.

### Table 137

The compounds in Table 137 are of the general formula (I) where Q1 is iodine, Q2, Q4, Q5 & Q6 are hydrogen, Q3 is bromine, m is 0, L is O, R¹ is methyl, and R² and R³ have the values given in the table. Thus, compound 1 of Table 137 is the same as compound 1 of Table 1 except that in compound 1 of Table 137, Q1 is iodine, Q3 is bromine. Similarly, compounds 2 to 299 of Table 137 are the same as compounds 2 to 299 of Table 1, respectively, except that in the compounds of Table 137, Q1 is iodine, Q3 is bromine.

### Table 138

The compounds in Table 138 are of the general formula (I) where Q1 is iodine, Q2, Q4, Q5 & Q6 are hydrogen, Q3 is bromine, m is 0, L is O, R¹ is ethyl, and R² and R³ have the values given in the table. Thus, compound 1 of Table 138 is the same as compound 1 of Table 1 except that in compound 1 of Table 138, Q1 is iodine, Q3 is bromine, R¹ is ethyl. Similarly, compounds 2 to 299 of Table 138 are the same as compounds 2 to 299 of Table 1, respectively, except that in the compounds of Table 138, Q1 is iodine, Q3 is bromine, R¹ is ethyl.

### Table 139

The compounds in Table 139 are of the general formula (I) where Q1 is iodine, Q2, Q4, Q5 & Q6 are hydrogen, Q3 is bromine, m is 1, L is O, R¹ is methyl, and R² and R³ have the values given in the table. Thus, compound 1 of Table 139 is the same as compound 1 of Table 1 except that in compound 1 of Table 139, Q1 is iodine, Q3 is bromine, m is 1. Similarly, compounds 2 to 299 of Table 139 are the same as compounds 2 to 299 of Table 1, respectively, except that in the compounds of Table 139, Q1 is iodine, Q3 is bromine, m is 1.

### Table 140

The compounds in Table 140 are of the general formula (I) where Q1 is iodine, Q2, Q4, Q5 & Q6 are hydrogen, Q3 is bromine, m is 1, L is O, R¹ is ethyl, and R² and R³ have the values given in the table. Thus, compound 1 of Table 140 is the same as compound 1 of Table 1 except that in compound 1 of Table 140, Q1 is iodine, Q3 is bromine, m is 1, R¹ is ethyl. Similarly, compounds 2 to 299 of Table 140 are the same as compounds 2 to 299 of Table 1, respectively, except that in the compounds of Table 140, Q1 is iodine, Q3 is bromine, m is 1, R¹ is ethyl.

### Table 141

The compounds in Table 141 are of the general formula (I) where Q1 is iodine, Q2, Q4, Q5 & Q6 are hydrogen, Q3 is methyl, m is 0, L is O, R¹ is methyl, and R² and R³ have the values given in the table. Thus, compound 1 of Table 141 is the same as compound 1 of Table 1 except that in compound 1 of Table 141, Q1 is iodine, Q3 is methyl. Similarly, compounds 2 to 299 of Table 141 are the same as compounds 2 to 299 of Table 1, respectively, except that in the compounds of Table 141, Q1 is iodine, Q3 is methyl.

### Table 142

The compounds in Table 142 are of the general formula (I) where Q1 is iodine, Q2, Q4, Q5 & Q6 are hydrogen, Q3 is methyl, m is 0, L is O, R¹ is ethyl, and R² and R³ have the values given in the table. Thus, compound 1 of Table 142 is the same as compound 1 of Table 1 except that in compound 1 of Table 142, Q1 is iodine, Q3 is methyl, R¹ is ethyl. Similarly, compounds 2 to 299 of Table 142 are the same as compounds 2 to 299 of Table 1, respectively, except that in the compounds of Table 142, Q1 is iodine, Q3 is methyl, R¹ is ethyl.

### Table 143

The compounds in Table 143 are of the general formula (I) where Q1 is iodine, Q2, Q4, Q5 & Q6 are hydrogen, Q3 is methyl, m is 1, L is O, R¹ is methyl, and R² and R³ have the values given in the table. Thus, compound 1 of Table 143 is the same as compound 1 of Table 1 except that in compound 1 of Table 143, Q1 is iodine, Q3 is methyl, m is 1. Similarly, compounds 2 to 299 of Table 143 are the same as compounds 2 to 299 of Table 1, respectively, except that in the compounds of Table 143, Q1 is iodine, Q3 is methyl, m is 1.

### Table 144

The compounds in Table 144 are of the general formula (I) where Q1 is iodine, Q2, Q4, Q5 & Q6 are hydrogen, Q3 is methyl, m is 1, L is O, R¹ is ethyl, and R² and R³ have the values given in the table. Thus, compound 1 of Table 144 is the same as compound 1 of Table 1 except that in compound 1 of Table 144, Q1 is iodine, Q3 is methyl, m is 1, R¹ is ethyl. Similarly, compounds 2 to 299 of Table 144 are the same as compounds 2 to 299 of Table 1, respectively, except that in the compounds of Table 144, Q1 is iodine, Q3 is methyl, m is 1, R¹ is ethyl.

### Table 145

The compounds in Table 145 are of the general formula (I) where Q1 is fluorine. Q2, Q3, Q4, Q5 & Q6 are hydrogen, m is 0, L is O, R¹ is methyl, and R² and R³ have the values given in the table. Thus, compound 1 of Table 145 is the same as compound 1 of Table 1 except that in compound 1 of Table 145, Q1 is fluorine. Similarly, compounds 2 to 299 of Table 145 are the same as compounds 2 to 299 of Table 1, respectively, except that in the compounds of Table 145, Q1 is fluorine.

### Table 146

The compounds in Table 146 are of the general formula (I) where Q1 is fluorine, Q2, Q3, Q4, Q5 & Q6 are hydrogen, m is 0, L is O, R¹ is ethyl, and R² and R³ have the values given in the table. Thus, compound 1 of Table 146 is the same as compound 1 of Table 1 except that in compound 1 of Table 146, Q1 is fluorine, R¹ is ethyl. Similarly, compounds 146 to 299 of Table 146 are the same as compounds 146 to 299 of Table 1, respectively, except that in the compounds of Table 146, Q1 is fluorine, R¹ is ethyl.

### Table 147

The compounds in Table 147 are of the general formula (I) where Q1 is fluorine, Q2, Q3, Q4, Q5 & Q6 are hydrogen, m is 1, L is O, R¹ is methyl, and R² and R³ have the values given in the table. Thus, compound 1 of Table 147 is the same as compound 1 of Table 1 except that in compound 1 of Table 147, Q1 is fluorine, m is 1. Similarly, compounds 2 to 299 of Table 147 are the same as compounds 2 to 299 of Table 1, respectively, except that in the compounds of Table 147, Q1 is fluorine, m is 1.

### Table 148

The compounds in Table 148 are of the general formula (I) where Q1 is fluorine, Q2, Q3, Q4, Q5 & Q6 are hydrogen, m is 1, L is O, R¹ is ethyl, and R² and R³ have the values given in the table. Thus, compound 1 of Table 148 is the same as compound 1 of Table 1 except that in compound 1 of Table 148, Q1 is fluorine, m is 1, R¹ is ethyl. Similarly, compounds 2 to 299 of Table 148 are the same as compounds 2 to 299 of Table 1, respectively, except that in the compounds of Table 148, Q1 is fluorine, m is 1, R¹ is ethyl.

### Table 149

The compounds in Table 149 are of the general formula (I) where Q1 is chlorine. Q2, Q3, Q4, Q5 & Q6 are hydrogen, m is 0, L is O, R¹ is methyl, and R² and R³ have the values given in the table. Thus, compound 1 of Table 149 is the same as compound 1 of Table 1 except that in compound 1 of Table 149, Q1 is chlorine. Similarly, compounds 2 to 299 of Table 149 are the same as compounds 2 to 299 of Table 1, respectively, except that in the compounds of Table 149, Q1 is chlorine.

### Table 150

The compounds in Table 150 are of the general formula (I) where Q1 is chlorine, Q2, Q3, Q4, Q5 & Q6 are hydrogen, m is 0, L is O, R¹ is ethyl, and R² and R³ have the values given in the table. Thus, compound 1 of Table 150 is the same as compound 1 of Table 1 except that in compound 1 of Table 150, Q1 is chlorine, R¹ is ethyl. Similarly, compounds 150 to 299 of Table 150 are the same as compounds 150 to 299 of Table 1, respectively, except that in the compounds of Table 150, Q1 is chlorine, R¹ is ethyl.

### Table 151

The compounds in Table 151 are of the general formula (I) where Q1 is chlorine, Q2, Q3, Q4, Q5 & Q6 are hydrogen, m is 1, L is O, R¹ is methyl, and R² and R³ have the values given in the table. Thus, compound 1 of Table 151 is the same as compound 1 of Table 1 except that in compound 1 of Table 151, Q1 is chlorine, m is 1. Similarly, compounds 2 to 299 of Table 151 are the same as compounds 2 to 299 of Table 1, respectively, except that in the compounds of Table 151, Q1 is chlorine, m is 1.

### Table 152

The compounds in Table 152 are of the general formula (I) where Q1 is chlorine, Q2, Q3, Q4, Q5 & Q6 are hydrogen, m is 1, L is O, R¹ is ethyl, and R² and R³ have the values given in the table. Thus, compound 1 of Table 152 is the same as compound 1 of Table 1 except that in compound 1 of Table 152, Q1 is chlorine, m is 1, R¹ is ethyl. Similarly, compounds 2 to 299 of Table 152 are the same as compounds 2 to 299 of Table 1, respectively, except that in the compounds of Table 152, Q1 is chlorine, m is 1, R¹ is ethyl.

### Table 153

The compounds in Table 153 are of the general formula (I) where Q1 is bromine. Q2, Q3, Q4, Q5 & Q6 are hydrogen, m is 0, L is O, R¹ is methyl, and R² and R³ have the values given in the table. Thus, compound 1 of Table 153 is the same as compound 1 of Table 1 except that in compound 1 of Table 153, Q1 is bromine. Similarly, compounds 2 to 299 of Table 153 are the same as compounds 2 to 299 of Table 1, respectively, except that in the compounds of Table 153, Q1 is bromine.

### Table 154

The compounds in Table 154 are of the general formula (I) where Q1 is bromine-Q2, Q3, Q4, Q5 & Q6 are hydrogen, m is 0, L is O, R¹ is ethyl, and R² and R³ have the values given in the table. Thus, compound 1 of Table 154 is the same as compound 1 of Table 1 except that in compound 1 of Table 154, Q1 is bromine, R¹ is ethyl. Similarly, compounds 154 to 299 of Table 154 are the same as compounds 154 to 299 of Table 1, respectively, except that in the compounds of Table 154, Q1 is bromine, R¹ is ethyl.

### Table 155

The compounds in Table 155 are of the general formula (I) where Q1 is bromine, Q2, Q3, Q4, Q5 & Q6 are hydrogen, m is 1, L is O, R¹ is methyl, and R² and R³ have the values given in the table. Thus, compound 1 of Table 155 is the same as compound 1 of Table 1 except that in compound 1 of Table 155, Q1 is bromine, m is 1. Similarly, compounds 2 to 299 of Table 155 are the same as compounds 2 to 299 of Table 1, respectively, except that in the compounds of Table 155, Q1 is bromine, m is 1.

### Table 156

The compounds in Table 156 are of the general formula (I) where Q1 is bromine, Q2, Q3, Q4, Q5 & Q6 are hydrogen, m is 1, L is O, R¹ is ethyl, and R² and R³ have the values given in the table. Thus, compound 1 of Table 156 is the same as compound 1 of Table 1 except that in compound 1 of Table 156, Q1 is bromine, m is 1, R¹ is ethyl. Similarly, compounds 2 to 299 of Table 156 are the same as compounds 2 to 299 of Table 1, respectively, except that in the compounds of Table 156, Q1 is bromine, m is 1, R¹ is ethyl.

### Table 157

The compounds in Table 157 are of the general formula (I) where Q1 is iodine. Q2, Q3, Q4, Q5 & Q6 are hydrogen, m is 0, L is O, R¹ is methyl, and R² and R³ have the values given in the table. Thus, compound 1 of Table 157 is the same as compound 1 of Table 1 except that in compound 1 of Table 157, Q1 is iodine. Similarly, compounds 2 to 299 of Table 157 are the same as compounds 2 to 299 of Table 1, respectively, except that in the compounds of Table 157, Q1 is iodine.

### Table 158

The compounds in Table 158 are of the general formula (I) where Q1 is iodine, Q2, Q3, Q4, Q5 & Q6 are hydrogen, m is 0, L is O, R¹ is ethyl, and R² and R³ have the values given in the table. Thus, compound 1 of Table 158 is the same as compound 1 of Table 1 except that in compound 1 of Table 158, Q1 is iodine, R¹ is ethyl. Similarly, compounds 158 to 299 of Table 158 are the same as compounds 158 to 299 of Table 1, respectively, except that in the compounds of Table 158, Q1 is iodine, R¹ is ethyl.

### Table 159

The compounds in Table 159 are of the general formula (I) where Q1 is iodine, Q2, Q3, Q4, Q5 & Q6 are hydrogen, m is 1, L is O, R¹ is methyl, and R² and R³ have the values given in the table. Thus, compound 1 of Table 159 is the same as compound 1 of Table 1 except that in compound 1 of Table 159, Q1 is iodine, m is 1. Similarly, compounds 2 to 299 of Table 159 are the same as compounds 2 to 299 of Table 1, respectively, except that in the compounds of Table 159, Q1 is iodine, m is 1.

### Table 160

The compounds in Table 160 are of the general formula (I) where Q1 is iodine, Q2, Q3, Q4, Q5 & Q6 are hydrogen, m is 1, L is O, R¹ is ethyl, and R² and R³ have the values given in the table. Thus, compound 1 of Table 160 is the same as compound 1 of Table 1 except that in compound 1 of Table 160, Q1 is iodine, m is 1, R¹ is ethyl. Similarly, compounds 2 to 299 of Table 160 are the same as compounds 2 to 299 of Table 1, respectively, except that in the compounds of Table 160, Q1 is iodine, m is 1, R¹ is ethyl.

The compounds of general formula (I) may be prepared as outlined in Schemes 1 to 9 below in which Q₁, Q₂, Q₃, Q₄, Q₅, Q₆, R₁, R₂, R₃, m and L have the meanings given above, R⁶ is H or C₁₋₄ alkyl, as indicated, R⁷, R⁸, R¹², R¹³ and R¹⁴ are independently H or C₁₋₄alkyl, R⁹ and R¹⁹ are independently H or C₁₋₄ alkyl where R⁹ and R¹⁰ are not simultaneously H, R¹³ and R¹⁴ are independently H or optionally substituted C₁₋₄ alkyl, R¹¹ is C₁₋₆ alkyl, optionally substituted benzyl, optionally substituted C₂₋₆ alkenyl, optionally substituted C₂₋₄ alkynyl, n is 0, 1 or 2 unless otherwise indicated. DMF is N,N-dimethylformamide, NBS is N-bromosuccinimide. Other abbreviations are defined in the text. Where typical or preferred process conditions (reaction temperature, time, solvent, mole ratios of reactants) are given, unless otherwise stated other process conditions can also be used. While optimum reaction conditions may vary with the particular reactants or solvents used, such conditions can be determined by routine optimisation procedures by one skilled in the art.

As shown in Scheme 1, the compounds of general formula (1) may be prepared by reacting a compound of the general formula (2) with a compound of the general formula (3) in the presence of a base in a suitable solvent. Typical solvents include N,N-dimethylformamide and N-methylpyrrolidin-2-one. Suitable bases include potassium carbonate, calcium carbonate, sodium hydride or diisopropylethylamine.

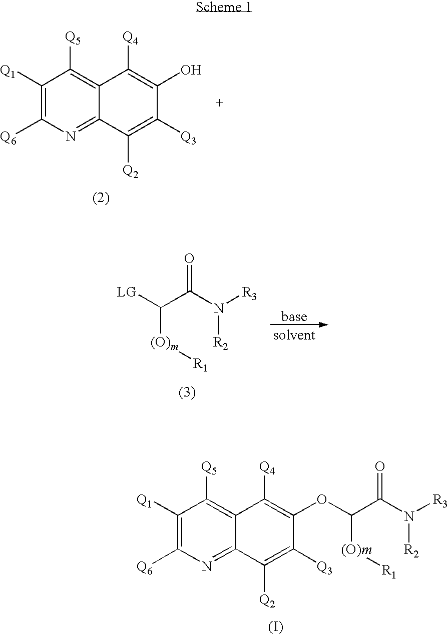

As shown in Scheme 2, compounds of the general formula (3) may be prepared by reacting an amine of the general formula (5) with an activated carboxylic acid such as an acid halide or the corresponding acid anhydride of the general formula (4), in the presence of a suitable inorganic or organic base, such as potassium carbonate or diisopropylethylamine, in a solvent such as dichloromethane, tetrahydrofuran or N,N-dimethylformamide.

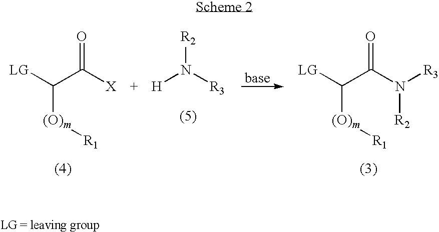

Alternatively, as shown in Scheme 3, compounds of the general formula (1) may be prepared by condensing a compound of the general formula (7a), wherein R₆ is H with an amine of the general formula (5) using suitable activating reagents such as 1-hydroxybenzotriazole (HOBt), (benzotriazol-1-yloxy)-tris-(dimethylamino)-phosphonium-hexa-fluorophosphate (BOP), 1-hydroxy-7-azabenzotriazole (HOAT) or, N-(3-dimethylamino-propyl)-N′-ethyl-carbodiimide hydrochloride (EDC).

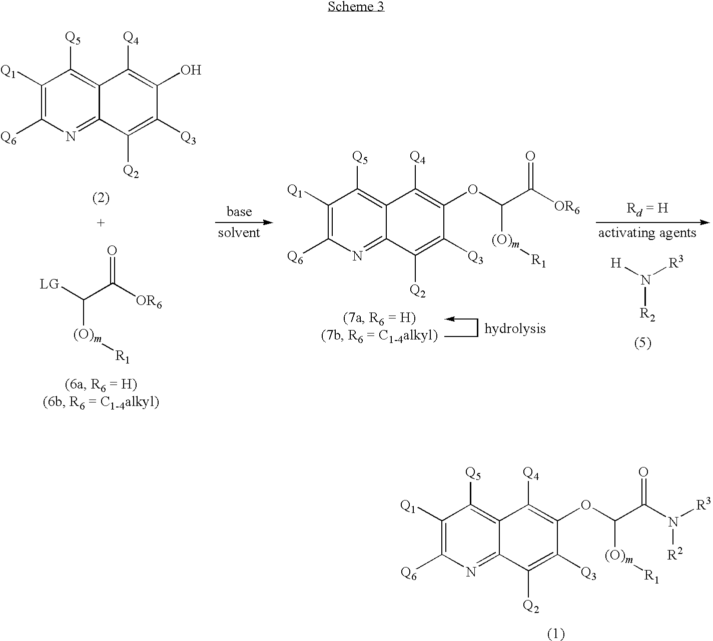

Compounds of the general formula (7a), wherein R₆ is H, may be prepared via the intermediacy of compounds of general formula (7b), wherein R₆ is C₁₋₄ alkyl as shown in Scheme 3. The esters of the general formula (7b), wherein R₆ is C₁₋₄ alkyl, may be prepared by reacting a compound of the general formula (2) with an ester of the general formula (6a) in the presence of a suitable base, such as potassium carbonate or sodium hydride, in a suitable solvent, such as N,N-dimethylformamide. Alternatively, compounds of the general formula (7a) wherein R₆ is H, may be prepared directly by reacting a compound of the general formula (2) with an acid of the general formula (6a) wherein R₆ is H. The esters or acids of the general formulae, (6a) and (6b) respectively, are either commercially available or may be prepared by standard literature methods from commercially available materials.

Alternatively, as shown in Scheme 4, compounds of the general formula (7b) may be prepared under Mitsunobu conditions by reacting a compound of the general formula (2) with a compound of the general formula (6b), wherein R₆ is C₁₋₄ alkyl, using a phosphine, such as triphenyl phosphine, and an azoester, such as diethyl azodicarboxylate.

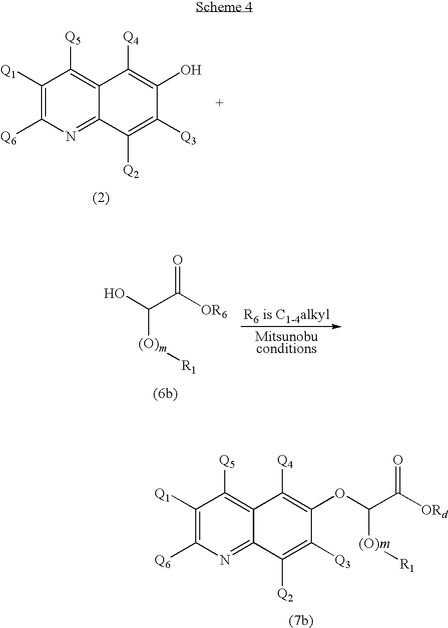

In another approach towards the preparation of compounds of the general formula (1) shown in Scheme 5, compound of general formula (6d) may be reacted with a compound of the general formula (2) under Mitsunobu conditions using a phosphine, such as triphenyl phosphine, and an azoester, such as diethyl azodicarboxylate. Compounds of general formula (6d) may be prepared from a compound of general formula (6c) and an amine of general formula (5) using suitable activating reagents such as 1-hydroxybenzotriazole and N-(3-dimethylaminopropyl)-N-ethyl-carbodiimide hydrochloride.

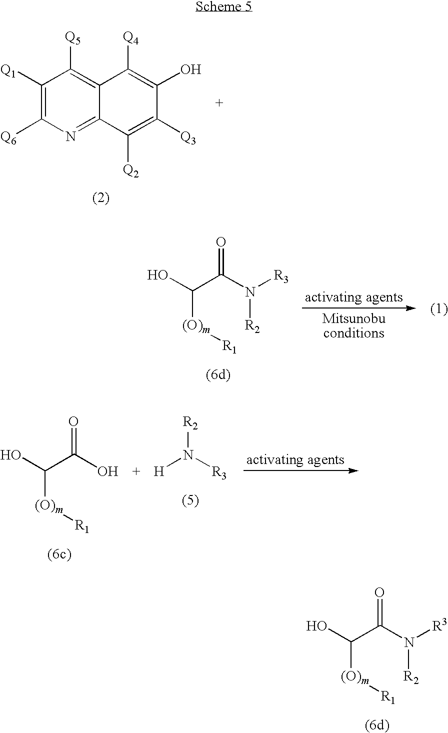

Compounds of general formula (6c) are either known compounds or may be prepared from commercially available and/or known compounds according to methods known to those skilled in the art.

In addition, compounds of the general formula (1) wherein m is 1, may be prepared as shown in Scheme 6. Thus, esters of the formula (8) may be halogenated to give haloesters of the general formula (9), by treatment with a suitable halogenating agent, such as N-bromosuccinimide, in a suitable solvent such as carbon tetrachloride, at between ambient temperature and the reflux temperature of the solvent. The haloesters of the general formula (9) can be reacted with an alkali metal compound MOR, where M is a metal such as sodium or potassium in solvent such as methanol or ethanol, between 0° C. and reflux, preferably at ambient temperature, to give compounds of the general formula (7b). The esters (7b) can be hydrolysed to acids of the general formula (7a), by treatment with an alkali metal hydroxide, such as sodium hydroxide, in aqueous alcohol, between ambient temperature and reflux. A carboxylic acid of the general formula (7a) can be condensed with an amine of the general formula (5) to give a compound of the general formula (1), where m is 1 and R₁ is as defined above, using suitable activating reagents such as 1-hydroxybenzotriazole and N-(3-dimethylaminopropyl)-N′-ethylcarbodiimide hydrochloride.

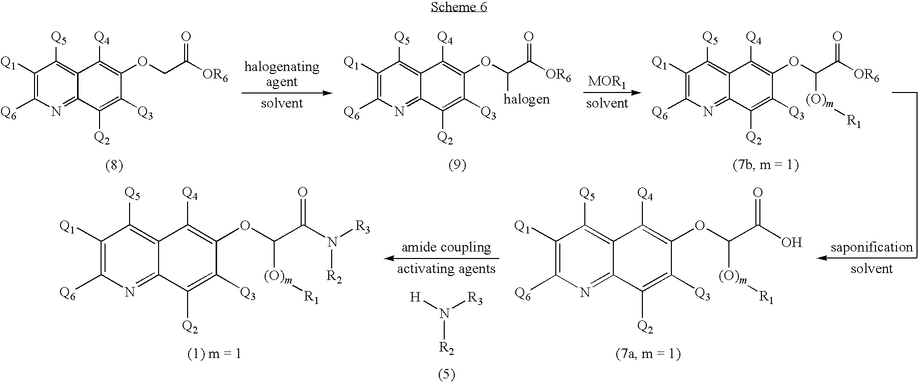

Compounds of the general formula (1), wherein m is 0, R₁ is C₁₋₄ alkyl, C₃₋₄ alkenyl, C₃₋₄ alkynyl, or an alkoxyalkyl group, may be prepared as shown in Scheme 7. Thus, the substituted acetic acid derivative (10) may be treated with at least two equivalents of a base, such as lithium diisopropylamide, in a suitable solvent such as tetrahydrofuran, at a temperature between −78° C. and ambient temperature, with an alkylating agent such as R₁L to give carboxylic acids of the general formula (7a) upon acidification.

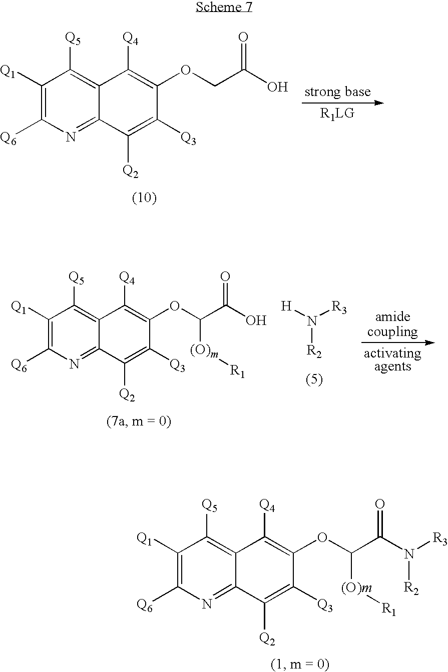

As shown in Scheme 8, amines of the general formula (9) or (11), which are examples of amines of the general formula (5) wherein R² is H, may be prepared by alkylation of an aminoalcohol of the general formula (8) or (10) using a suitable base, such as n-butyl lithium or sodium hydride, followed by reaction with a suitable alkylating reagent R¹¹LG, such as an alkyl iodide, for example, methyl iodide, to form an alkylated compound of the general formula (9) or (11), respectively. A carbonyl derivative R¹³COR¹⁴ (12), for example formaldehyde, can be reacted with ammonia, usually in form of ammonium chloride, and cyanide, conveniently in form of an aqueous solution sodium cyanide, to provide an α-cyanoamine (13) (Strecker synthesis).

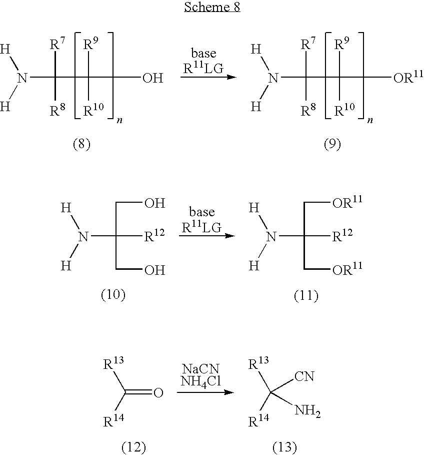

Alternatively, as shown in Scheme 9, amines of the general formula (16), which are examples of amines of the general formula (5) wherein R² is H, may be prepared by nucleophilic opening of cyclic sulphamidates of the general formula (14), where PG is a protecting group such as tert-butyl carbamate (BOC) or 4-methoxy benzyl (PMB), using a variety of nucleophiles, such as the tetra n-butyl ammonium fluoride (TBAF) and sodium cyanide followed by deprotection using standard conditions (such as HCl for removal of the BOC group or hydrogen and a palladium-based catalyst for the removal of the PMB group).

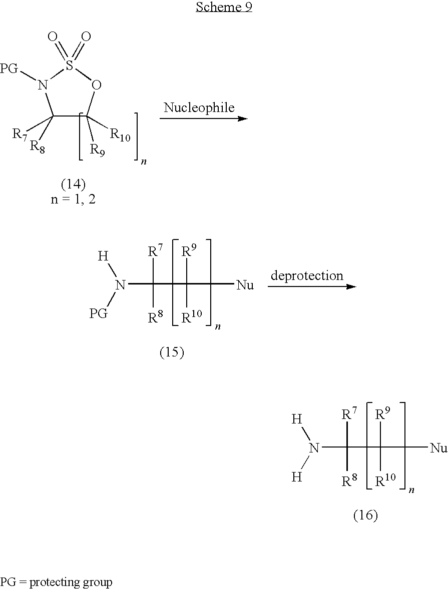

Compounds of the formula (14) may be prepared by methods known to those skilled in the art (for example as described in the Journal of the American Chemical Society, 2001, pages 6935-6936).

In addition, as shown in Scheme 10, hydrolysis of the cyclic sulphamidate (14) provides the N-protected amino alcohol (15) which can be further modified by methods known to those skilled in the art. For example, deprotection of compounds of the general formula (15) using standard conditions (such as HCl for removal of the BOC group or hydrogen and a palladium-based catalyst for the removal of the PMB group) provides amino alcohols of the general formula (8) which are examples of amines of the general formula (5) wherein R² is H. Alternatively, when one of R⁹ and R¹⁰ is hydrogen, oxidation of compounds of the formula (15) with oxidants such as hypervalent iodine reagents, for example Dess-Martin periodinane provides oxidised compounds of the formula (17) which are examples of amines of the general formula (5) wherein R² is H after deprotection of compounds of the formula (16) using standard conditions known to those skilled in the art.

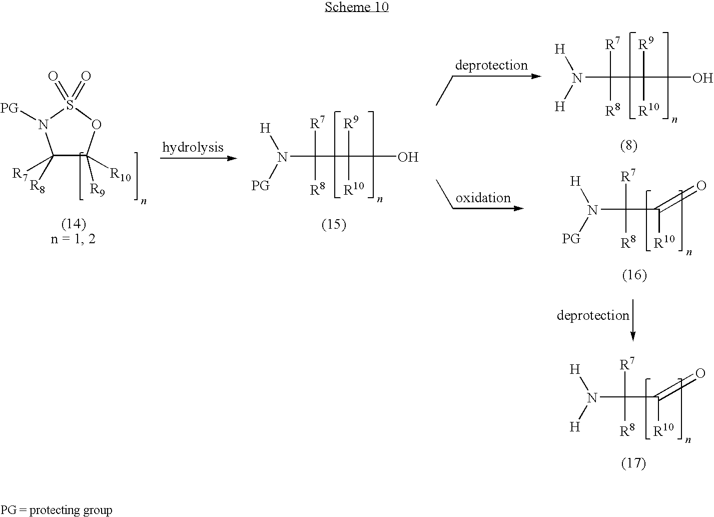

Other derivatives of the amines of the general formula (5) may be prepared by derivatisation of the compounds of the general formulae (15) and (16) using standard procedures known to those skilled in the art.

Other amines of the general formula (5) are either commercially available or may be prepared by standard literature methods or standard modifications known to those skilled in the art.

Thioamides (Compounds of the general formula (1) where L=S) may be prepared from the corresponding amides using thionating agents such as phosphorous pentasulphide, Lawesson's or Davy's reagents or prepared from the corresponding thionoacids or thionoesters using standard literature methods or standard modifications.

Quinolines are commercially available or may be prepared using methods known to those skilled in the art that are well described in standard textbooks of heterocyclic chemistry and in the literature. For example, two references describe the synthesis of 3 and/or 8 substituted quinolines: Journal of the American Chemical Society (1955), 77, 4175 and Journal of the American Chemical Society (1950), 72 393. Numerous synthetic routes to the 6-hydroxy quinolines of the general formula (2) of the present invention can thus be devised by any person skilled in the art. Specific examples of such reactions are provided in Examples 1, 5 and 6 so as to allow one skilled in the art to make and use of quinolines of the invention. It is understood that these examples serve in no way to limit the true scope of this invention but rather are presented for illustrative purposes.

Other compounds of the invention may be prepared by transforming the substituents in the compounds of the general formula (1) using standard procedures known to those skilled in the art.

The compounds of formula (I) are active fungicides and may be used to control one or more of the following pathogens: Pyricularia oryzae (Magnaporthe grisea) on rice and wheat and other Pyricularia spp. on other hosts; Puccinia triticina (or recondita), Puccinia striiformis and other rusts on wheat, Puccinia hordei, Puccinia striiformis and other rusts on barley, and rusts on other hosts (for example turf, rye, coffee, pears, apples, peanuts, sugar beet, vegetables and ornamental plants); Erysiphe cichoracearum on cucurbits (for example melon); Blumeria (or Erysiphe) graminis (powdery mildew) on barley, wheat, rye and turf and other powdery mildews on various hosts, such as Sphaerotheca macularis on hops, Sphaerotheca fusca (Sphaerotheca fuliginea) on cucurbits (for example cucumber), Leveifiula taurica on tomatoes, aubergine and green pepper, Podosphaera leucotricha on apples and Uncinula necator on vines; Cochliobolus spp., Helminthosporium spp., Drechslera spp. (Pyrenophora spp.), Rhynchosporium spp., Mycosphaerella graminicola (Septoria tritici) and Phaeosphaeria nodorum (Stagonospora nodorum or Septoria nodorum), Pseudocercosporella herpotrichoides and Gaeumannomyces graminis on cereals (for example wheat, barley, rye), turf and other hosts; Cercospora arachidicola and Cercosporidium personatum on peanuts and other Cercospora spp. on other hosts, for example sugar beet, bananas, soya beans and rice; Botrytis cinerea (grey mould) on tomatoes, strawberries, vegetables, vines and other hosts and other Bottytis spp. on other hosts; Alternaria spp. on vegetables (for example carrots), oil-seed rape, apples, tomatoes, potatoes, cereals (for example wheat) and other hosts; Venturia spp. (including Venturia inaequalis (scab)) on apples, pears, stone fruit, tree nuts and other hosts; Cladosporium spp. on a range of hosts including cereals (for example wheat) and tomatoes; Monilinia spp. on stone fruit, tree nuts and other hosts; Didymella spp. on tomatoes, turf, wheat, cucurbits and other hosts; Phoma spp. on oil-seed rape, turf, rice, potatoes, wheat and other hosts; Aspergillus spp. and Aureobasidium spp. on wheat, lumber and other hosts; Ascochyta spp. on peas, wheat, barley and other hosts; Stemphylium spp. (Pleospora spp.) on apples, pears, onions and other hosts; summer diseases (for example bitter rot (Glomerella cingulata), black rot or frogeye leaf spot (Botryosphaeria obtusa), Brooks fruit spot (Mycosphaerella pomi), Cedar apple rust (Gymnosporangium juniperi-virginianae), sooty blotch (Gloeodes pomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. on soybeans, tobacco, onions and other hosts, Pseudoperonospora humuli on hops and Pseudoperonospora cubensis on cucurbits; Pythium spp. (including Pythium ultimum) on turf and other hosts; Phytophthora infestans on potatoes and tomatoes and other Phytophthora spp. on vegetables, strawberries, avocado, pepper, ornamentals, tobacco, cocoa and other hosts; Thanatephorus cucumeris on rice and turf and other Rhizoctonia spp. on various hosts such as wheat and barley, peanuts, vegetables, cotton and turf; Sclerotinia spp. on turf, peanuts, potatoes, oil-seed rape and other hosts; Sclerotium spp. on turf, peanuts and other hosts; Gibberella fujikuroi on rice; Colletotrichum spp. on a range of hosts including turf, coffee and vegetables; Laetisaria fuciformis on turf; Mycosphaerella spp. on bananas, peanuts, citrus, pecans, papaya and other hosts; Diaporthe spp. on citrus, soybean, melon, pears, lupin and other hosts; Elsinoe spp. on citrus, vines, olives, pecans, roses and other hosts; Verticillium spp. on a range of hosts including hops, potatoes and tomatoes; Pyrenopeziza spp. on oil-seed rape and other hosts; Oncobasidium theobromae on cocoa causing vascular streak dieback; Fusarium spp., Typhula spp., Microdochium nivale, Ustilago spp., Urocystis spp., Tilletia spp. and Claviceps purpurea on a variety of hosts but particularly wheat, barley, turf and maize; Ramularia spp. on sugar beet, barley and other hosts; post-harvest diseases particularly of fruit (for example Penicillium digitatum, Penicillium italicum and Trichoderma viride on oranges, Colletotrichum musae and Gloeosporium musarum on bananas and Botrytis cinerea on grapes); other pathogens on vines, notably Eutypa lata, Guignardia bidwellii, Phellinus igniarus, Phomopsis viticola, Pseudopeziza tracheiphila and Stereum hirsutum; other pathogens on trees (for example Lophodermium seditiosum) or lumber, notably Cephaloascus fragrans, Ceratocystis spp., Ophiostoma piceae, Penicillium spp., Trichoderma pseudokoningii, Trichoderma viride, Trichoderma harzianum, Aspergillus niger, Leptographium lindbergi and Aureobasidium pullulans; and fungal vectors of viral diseases (for example Polymyxa graminis on cereals as the vector of barley yellow mosaic virus (BYMV) and Polymyxa betae on sugar beet as the vector of rhizomania).

A compound of formula (I) may move acropetally, basipetally or locally in plant tissue to be active against one or more fungi. Moreover, a compound of formula (I) may be volatile enough to be active in the vapour phase against one or more fungi on the plant.

The invention therefore provides a method of combating or controlling phytopathogenic fungi which comprises applying a fungicidally effective amount of a compound of formula (I), or a composition containing a compound of formula (I), to a plant, to a seed of a plant, to the locus of the plant or seed or to soil or any other plant growth medium, e.g. nutrient solution.

The term “plant” as used herein includes seedlings, crops of useful plants, bushes and trees. Furthermore, the fungicidal method of the invention includes protectant, curative, systemic, eradicant and antisporulant treatments.

Crops of useful plants in which the compositions according to the invention can be used include especially cereals, maize, rice, rape, sugar beet, sugar cane, plantations, cotton, soybeans, vegetables and flowers. The term “crops” is to be understood as also including crops that have been rendered tolerant to herbicides or classes of herbicides (for example ALS, GS, EPSPS, PPO and HPPD inhibitors) as a result of conventional methods of breeding or genetic engineering. An example of a crop that has been rendered tolerant e.g. to imidazolinones, such as imazamox, by conventional methods of breeding is Clearfield® summer rape (Canola). Examples of crops that have been rendered tolerant to herbicides by genetic engineering methods include e.g. glyphosate- and glufosinate-resistant maize varieties commercially available under the trade names RoundupReady® and LibertyLink®. The weeds to be controlled may be both monocotyledonous and dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus, Abutilon, Sida, Xanthium, Amaranthus, Chenopodium, Ipomoea, Chrysanthemum, Galium, Viola and Veronica.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt-176 maize hybrids of NK® (Syngenta Seeds). The Bt toxin is a protein that is formed naturally by Bacillus thuringiensis soil bacteria. Examples of toxins and transgenic plants able to synthesise such toxins are described in EP-A-451 878, EP-A-374 753, WO 93/07278, WO 95/34656, WO 03/052073 and EP-A-427 529. Examples of transgenic plants that contain one or more genes which code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops and their seed material can be resistant to herbicides and at the same time also to insect feeding (“stacked” transgenic events). Seed can, for example, have the ability to express an insecticidally active Cry3 protein and at the same time be glyphosate-tolerant. The term “crops” is to be understood as also including crops obtained as a result of conventional methods of breeding or genetic engineering which contain so-called output traits (e.g. improved flavour, storage stability, nutritional content).

Areas under cultivation are to be understood as including land where the crop plants are already growing as well as land intended for the cultivation of those crop plants.

The compounds of formula (I) are preferably used for agricultural, horticultural and turfgrass purposes in the form of a composition.

In order to apply a compound of formula (I) to a plant, to a seed of a plant, to the locus of the plant or seed or to soil or any other growth medium, a compound of formula (I) is usually formulated into a composition which includes, in addition to the compound of formula (I), a suitable inert diluent or carrier and, optionally, a surface active agent (SFA). SFAs are chemicals that are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of fungi such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides a fungicidal composition comprising a fungicidally effective amount of a compound of formula (I) and a suitable carrier or diluent therefor.

In a still further aspect the invention provides a method of combating and controlling fungi at a locus, which comprises treating the fungi, or the locus of the fungi with a fungicidally effective amount of a composition comprising a compound of formula (I). The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone), alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at ambient temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents that have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurised, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier). Wetting agents, dispersing agents and emulsifying agents may be SFAs of the cationic, anionic, amphoteric or non-ionic type.

Suitable SFAs of the cationic type include quaternary ammonium compounds (for example acetyltrimethyl ammonium bromide), imidazolines and amine salts. Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefin sulphonates, taurates and lignosulphonates. Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying fungicidal compounds. For example, it may be applied, formulated or unformulated, to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilisers (for example nitrogen-, potassium- or phosphorus-containing fertilisers). Suitable formulation types include granules of fertiliser. The mixtures suitably contain up to 25% by weight of the compound of formula (I).

The invention therefore also provides a fertiliser composition comprising a fertiliser and a compound of formula (I).

The compositions of this invention may contain other compounds having biological activity, for example micronutrients or compounds having similar or complementary fungicidal activity or which possess plant growth regulating, herbicidal, insecticidal, nematicidal or acaricidal activity.

By including another fungicide, the resulting composition may have a broader spectrum of activity or a greater level of intrinsic activity than the compound of formula (I) alone. Further the other fungicide may have a synergistic effect on the fungicidal activity of the compound of formula (I).

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I); or help to overcome or prevent the development of resistance to individual components. The particular additional active ingredient will depend upon the intended utility of the composition.

Examples of fungicidal compounds which may be included in the composition of the invention are AC 382042 (N-(1-cyano-1,2-dimethylpropyl)-2-(2,4-dichlorophenoxy) propionamide), acibenzolar-S-methyl, alanycarb, aldimorph, anilazine, azaconazole, azafenidin, azoxystrobin, benalaxyl, benomyl, benthiavalicarb, biloxazol, bitertanol, blasticidin S, boscalid (new name for nicobifen), bromuconazole, bupirimate, captafol, captan, carbendazim, carbendazim chlorhydrate, carboxin, carpropamid, carvone, CGA 41396, CGA 41397, chinomethionate, chlorbenzthiazone, chlorothalonil, chlorozolinate, clozylacon, copper containing compounds such as copper oxychloride, copper oxyquinolate, copper sulphate, copper tallate, and Bordeaux mixture, cyamidazosulfamid, cyazofamid (IKF-916), cyflufenamid, cymoxanil, cyproconazole, cyprodinil, debacarb, di-2-pyridyl disulphide 1,1′-dioxide, dichlofluanid, diclocymet, diclomezine, dicloran, diethofencarb, difenoconazole, difenzoquat, diflumetorim, O,O-di-iso-propyl-5-benzyl thiophosphate, dimefluazole, dimetconazole, dimethirimol, dimethomorph, dimoxystrobin, diniconazole, dinocap, dithianon, dodecyl dimethyl ammonium chloride, dodemorph, dodine, doguadine, edifenphos, epoxiconazole, ethaboxam, ethirimol, ethyl (Z)-N-benzyl-N([methyl(methyl-thioethylideneaminooxycarbonyl)amino]thio)-β-alaninate, etridiazole, famoxadone, fenamidone, fenarimol, fenbuconazole, fenfuram, fenhexamid, fenoxanil (AC 382042), fenpiclonil, fenpropidin, fenpropimorph, fentin acetate, fentin hydroxide, ferbam, ferimzone, fluazinam, fludioxonil, flumetover, flumorph, fluoroimide, fluoxastrobin, fluquinconazole, flusilazole, flusulfamide, flutolanil, flutriafol, folpet, fosetyl-aluminium, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb, isopropanyl butyl carbamate, isoprothiolane, kasugamycin, kresoxim-methyl, LY186054, LY211795, LY 248908, mancozeb, maneb, mefenoxam, mepanipyrim, mepronil, metalaxyl, metalaxyl M, metconazole, metiram, metiram-zinc, metominostrobin, metrafenone, MON65500 (N-allyl-4,5-dimethyl-2-trimethylsilylthiophene-3-carboxamide), myclobutanil, NTN0301, neoasozin, nickel dimethyldithiocarbamate, nitrothale-isopropyl, nuarimol, ofurace, organomercury compounds, orysastrobin, oxadixyl, oxasulfuron, oxolinic acid, oxpoconazole, oxycarboxin, pefurazoate, penconazole, pencycuron, phenazin oxide, phosphorus acids, phthalide, picoxystrobin, polyoxin D, polyram, probenazole, prochloraz, procymidone, propamocarb, propamocarb hydrochloride, propiconazole, propineb, propionic acid, proquinazid, prothioconazole, pyraclostrobin, pyrazophos, pyrifenox, pyrimethanil, pyroquilon, pyroxyfur, pyrroInitrin, quaternary ammonium compounds, quinomethionate, quinoxyfen, quintozene, silthiofam (MON 65500), S-imazalil, simeconazole, sipconazole, sodium pentachlorophenate, spiroxamine, streptomycin, sulphur, tebuconazole, tecloftalam, tecnazene, tetraconazole, thiabendazole, thifluzamide, 2-(thiocyanomethylthio)benzothiazole, thiophanate-methyl, thiram, tiadinil, timibenconazole, tolclofos-methyl, tolylfluanid, triadimefon, triadimenol, triazbutil, triazoxide, tricyclazole, tridemorph, trifloxystrobin, triflumizole, triforine, triticonazole, validamycin A, vapam, vinclozolin, XRD-563, zineb, ziram, zoxamide and the compounds of the formulae:

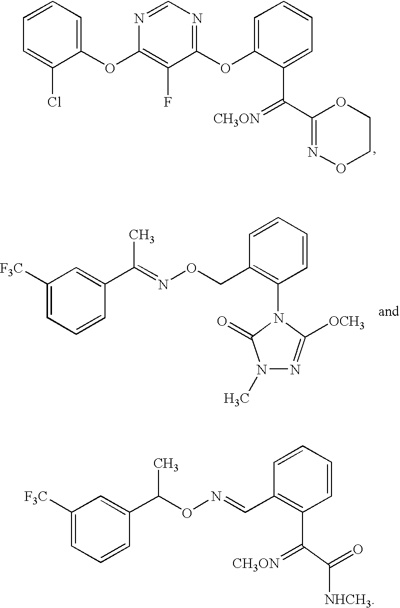

The compounds of formula (I) may be mixed with soil, peat or other rooting media for the protection of plants against seed-borne, soil-borne or foliar fungal diseases. Some mixtures may comprise active ingredients, which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following examples in which the following abbreviations are used:
ml=millilitres
g=grammes
ppm=parts per million
M⁺=mass ion
s=singlet
d=doublet
br s=broad singlet
t=triplet
DMSO=dimethylsulphoxide
NMR=nuclear magnetic resonance
HPLC=high performance liquid chromatography
q=quartet
m=multiplet

### EXAMPLE 1

This example illustrates the preparation of 2-(3-bromo-quinolin-6-yloxy)-N-tert-butyl-butyramide (Compound No. 12 of Table 154) according to Scheme 3 wherein m=0.

### Step 1: Preparation of 2-(3-Bromo-quinolin-6-yloxy)-butyric acid ethyl ester

3-Bromo-quinolin-6-ol (11.3 g) (preparation described in Liebigs Ann Chem 1966, 98-106) was dissolved in dry DMF (100 ml). 2-Bromo-butyric acid ethyl ester (11.05 g) and dry potassium carbonate (20.9 g) were added to the mixture at R.T. The resulting suspension was stirred at 70° C. for 1 hour. The reaction mixture was poured into brine. After removal of the precipitate and separation of the two phases, the aqueous layer was extracted twice with ethyl acetate (2×100 ml). The organic layers were combined, washed with brine, dried over magnesium sulphate, filtered and evaporated to give 2-(3-bromo-quinolin-6-yloxy)-butyric acid ethyl ester as a oil (17.8) which was used in the next step without further purification.

¹H NMR (CDCl₃) δ ppm: 8.78 (1H, d); 8.15 (1H, d); 7.98 (1H, d); 7.42 (1H, d×d); 6.90 (1H, d); 4.70 (1H, t); 4.25 (2H, q); 2.08 (2H, m); 1.25 (3H, t); 1.13 (3H, t).

### Step 2: Preparation of 2-(3-Bromo-quinolin-6-yloxy)-butyric acid

To a suspension of 2-(3-Bromo-quinolin-6-yloxy)-butyric acid ethyl ester (17.8 g) in a mixture (500 ml) of tetrahydrofurane/water (1/1) at zero ° C. is added lithium hydroxide monohydrate (2.31 g). The reaction mixture is allowed to warm up to ambient temperature and is stirred overnight. Ethyl acetate was added and the two phases were separated. The aqueous phase was acidified with dilute aqueous hydrochloric acid then extracted twice with ethyl acetate. The organic phases were combined, dried over magnesium sulphate, filtered and evaporated to give 2-(3-bromo-quinolin-6-yloxy)-butyric acid as a brown powder (13.1) which was used in the next step without further purification.

¹H NMR (CDCl₃) δ ppm: 8.70 (1H, d); 8.13 (1H, d); 7.92 (1H, d); 7.35 (1H, d×d); 6.90 (1H, d); 4.70 (1H, t); 3.3-2.5 (1H, br); 2.05 (2H, m); 1.10 (3H, t).

### Step 3: Preparation of 2-(3-Bromo-quinolin-6-yloxy)-N-tert-butyl-butyramide

2-(3-Bromo-quinolin-6-yloxy)-butyric acid (0.209 g), N-tert-butyl amine (0.074 ml), 1-hydroxy-7-azabenzotriazole (HOAT) (0.097 g), N-(3-dimethylaminopropyl)-N′-ethylcarbodiimide hydrochloride (EDC) (0.137 g) and triethylamine (0.098 ml) in dry N,N-dimethylformamide (7 ml) were stirred at ambient temperature for 3.5 hours. The reaction mixture was quenched with water and poured onto a Chromabond-XTR cartridge followed by elution with ethyl acetate (130 ml). The crude mixture was purified by reverse phase HPLC (acetonitrile-water) to give 2-(3-bromo-quinolin-6-yloxy)-N-tert-butyl-butyramide as an oil (0.16 g).

¹H NMR (CDCl₃) δ ppm: 8.70 (1H, d); 8.12 (1H, d); 7.92 (1H, d); 7.32 (1H, d×d); 6.92 (1H, d); 6.05 (1H, br s); 4.43 (1H, m); 1.92 (2H, m); 1.20 (9H, s); 0.98 (3H, t).

The following amides were prepared using a similar procedure.

Compound No. 47 of Table 154: 2-(3-Bromo-quinolin-6-yloxy)-N-(2-methoxy-1,1-dimethyl-ethyl)-butyramide using 2-methoxy-1,1-dimethyl-ethylamine.

¹H NMR (CDCl₃) δ ppm: 8.75 (1H, d); 8.15 (1H, d); 7.96 (1H, d); 7.38 (1H, d×d); 7.0 (1H, d); 6.42 (1H, br s); 4.50 (1H, m); 3.25 (2H, m); 3.23 (3H, s); 2.02 (2H, m); 1.31 (3H, s); 1.24 (3H, s); 1.05 (3H, t).

Compound No. 52 of Table 154: 2-(3-Bromo-quinolin-6-yloxy)-N-(1-cyano-2-methoxy-1-methyl-ethyl)-butyramide using 2-amino-3-methoxy-2-methyl-propionitrile:

¹H NMR (CDCl₃) δ ppm: diastereoisomeric mixture (9/1); major diastereoisomer: 8.72 (1H, d); 8.18 (1H, d×m); 7.95 (1H, d); 7.35 (1H, d×d); 6.98 (1H, m); 6.88 (1H, m br); 4.65 (1H, m); 3.6 (2H, m); 3.25 (3H, s); 2.05 (2H, m); 1.70 (3H, s); 1.05 (3H, t).

2-(3-Bromo-quinolin-6-yloxy)-N-tert-butyl-3-methyl-butyramide using 2-(3-bromo-quinolin-6-yloxy)-3-methyl-butyric acid and N-tert-butylamine. M.p. (° C.): 123-125; ¹H NMR (CDCl₃) δ ppm: 8.78 (1H, d); 8.18 (1H, d); 8.01 (1H, d); 7.41 (1H, d×d); 7.02 (1H, d); 5.59 (1H, br s); 4.32 (1H, d); 2.3 (1H, m); 1.30 (9H, s); 1.09 (6H, m).

2-(3-Bromo-quinolin-6-yloxy)-N-(1-cyano-2-methoxy-1-methyl-ethyl)-3-methyl-butyramide using 2-(3-bromo-quinolin-6-yloxy)-3-methyl-butyric acid and 2-cyano-1-methoxy-prop-2-ylamine. M.p. (° C.): 151-157; ¹H NMR (CDCl₃) δ ppm: diastereoisomeric mixture (1/1); 8.80 (1H, d); 8.20 (1H, d); 8.02 (1H, d); 7.41 (1H, d×d); 7.02 (1H, m); 6.67 (1H, br m); 4.48 (1H, m); 3.68-3.45 (2H, m+s); 3.41 and 3.21 (3H, 2×s); 2.36 (1H, m); 1.70- and 1.61 (3H, 2×s); 1.10 (6H, m).

2-(3-Bromo-quinolin-6-yloxy)-N-tert-butyl-2-cyclopropyl-acetamide using (3-bromo-quinolin-6-yloxy)-cyclopropyl acetic acid and N-tert-butylamine. M.p. (° C.): 165-167; ¹H NMR (CDCl₃) δ ppm: 8.78 (1H, d); 8.18 (1H, d); 8.00 (1H, d); 7.39 (1H, d×d); 6.98 (1H, d); 5.99 (1H, br s); 4.15 (1H, d); 1.3 (1H, m); 1.29 (9H, s); 0.75-0.50 (4H, 2×m).

2-(3-Bromo-quinolin-6-yloxy)-2-cyclopropyl-N-(2-methoxy-1,1-dimethyl-ethyl)-acetamide using (3-bromo-quinolin-6-yloxy)-cyclopropyl acetic acid and 2-methoxy-1,1-dimethyl-ethylamine. M.p. (° C.): 139-141; ¹H NMR (CDCl₃) δ ppm: 8.77 (1H, d); 8.18 (1H, d); 7.99 (1H, d); 7.40 (1H, d×d); 6.98 (1H, d); 6.33 (1H, br s); 4.15 (1H, d); 3.33-3.18 (2H, m); 3.22 (3H, s); 1.35 (1H, m); 1.31 (3H, s); 1.24 (3H, s); 0.75-0.47 (4H, 2×m).

2-(3-Bromo-quinolin-6-yloxy)-N-(1-cyano-2-methoxy-1-methyl-ethyl)-2-cyclopropyl-acetamide using (3-Bromo-quinolin-6-yloxy)-cyclopropyl acetic acid and 2-cyano-1-methoxy-prop-2-ylamine. M.p. (° C.): 119-121; ¹H NMR (CDCl₃) δ ppm: diastereoisomeric mixture (1/1); 8.79 (1H, d); 8.20 (1H, m); 8.01 (1H, d); 7.40 (1H, d×d); 6.98 (1H, m); 6.74 and 6.68 (1H, 2×br s); 4.29 (1H, m); 3.70-3.48 (2H, m+s); 3.42 and 3.23 (3H, 2×s); 1.72 and 1.62 (3H, 2×s); 1.38 (1H, m); 0.80-0.50 (4H, 2×m).

### EXAMPLE 2

This Example illustrates the preparation of 2-(3-bromo-quinolin-6-yloxy)-N-tert-butyl-2-methoxy-acetamide (Compound No. 12 of Table 155) according to Scheme 6 wherein m=1.

### Step 1: Preparation of (3-Bromo-quinolin-6-yloxy)acetic acid methyl ester

3-Bromo-quinolin-6-ol (10.0 g) was dissolved in dry DMF (100 ml). Bromo-acetic acid methyl ester (7.51 g) and dry potassium carbonate (18.5 g) were added to the mixture at R.T. The resulting suspension was stirred at 80° C. for 2 hours. The reaction mixture was poured onto brine and the resulting mixture was extracted twice with ethyl acetate (2×100 ml). The organic layers were combined, washed with brine, dried over magnesium sulphate, filtered and evaporated. The crude mixture was recrystallized in isopropanol to give (3-bromo-quinolin-6-yloxy)acetic acid methyl ester (11.5 g) as a pale yellow solid.

¹H NMR (CDCl₃) δ ppm: 8.78 (1H, d); 8.20 (1H, d), 8.0 (1H, d); 7.44 (1H, d×d); 6.92 (1H, d); 4.76 (2H, s); 3.85 (3H, s).

### Step 2: Preparation of bromo-(3-bromo-quinolin-6-yloxy)acetic acid methyl ester

A suspension of (3-bromo-quinolin-6-yloxy)acetic acid methyl ester (2.0 g), N-bromosuccinimide (1.80 g) and, Azo-isobutyronitrile (0.222 g) in carbon tetrachloride (100 ml) was heated up at reflux for 3 hrs. The reaction mixture was cooled down to ambient temperature, filtered off and concentrated in vacuum to give bromo-(3-bromo-quinolin-6-yloxy)acetic acid methyl ester (2.8 g) as yellow solid. The crude mixture was used in the next step without further purification.

¹H NMR (CDCl₃) δ ppm: 8.87 (1H, d); 8.35 (1H, d); 8.12 (1H, d); 7.55 (1H, d×d); 7.41 (1H, d); 6.62 (1H, s); 3.98 (3H, s).

### Step 3: Preparation of (3-bromo-quinolin-6-yloxy)methoxy acetic acid methyl ester

To a suspension of bromo-(3-bromo-quinolin-6-yloxy)acetic acid methyl ester (1.32 g) in anhydrous methanol was added Calcium carbonate (1.05 g). The reaction mixture was stirred at ambient temperature for 2.5 hrs; treated with chloroform and further stirred at R.T. for 10 minutes. The mixture was filtered off and the solvent was evaporated. The resulting crude oil was fractionated by reverse phase HPLC (eluant: acetonitrile/water) to give (3-bromo-quinolin-6-yloxy)methoxy acetic acid methyl ester (0.5 g) as a yellow oil.

¹H NMR (CDCl₃) δ ppm: 8.80 (1H, d); 8.22 (1H, d); 8.02 (1H, d); 7.51 (1H, d×d); 7.30 (1H, d); 5.63 (1H, s); 3.87 (3H, s); 3.58 (3H, s).

### Step 4: Preparation of (3-bromo-quinolin-6-yloxy)methoxy acetic acid

A suspension of (3-bromo-quinolin-6-yloxy)methoxy acetic acid methyl ester (0.495 g) and lithium hydroxide monohydrate (0.070 g) in a mixture (500 ml) of tetrahydrofurane/water (1/1) was stirred at zero ° C. for 2.5 hrs. The reaction mixture is allowed to warm up to ambient temperature; ethyl acetate was added and the two phases were separated. The aqueous phase was acidified with dilute aqueous hydrochloric acid then extracted twice with ethyl acetate. The organic phases were combined, dried over magnesium sulphate, filtered and evaporated to give (3-bromo-quinolin-6-yloxy)methoxy acetic acid (0.39 g) as a white solid which was used in the next step without further purification.

¹H NMR (DMSO d₆) δ ppm: 8.82 (1H, d); 8.63 (1H, d); 7.99 (1H, d); 7.57 (1H, d×d); 7.50 (1H, d); 5.78 (1H, s); 3.46 (3H, s).

### Step 5: Preparation of 2-(3-bromo-quinolin-6-yloxy)-N-tert-butyl-2-methoxy acetamide

2-(3-bromo-quinolin-6-yloxy)-butyric acid (0.052 g), N-tert-Butyl amine (0.024 g), (benzotriazol-1-yloxy)-tris-(dimethylamino)-phosphonium-hexafluorophosphate (BOP) (0.099 g), N-Ethyldiisopropylamine (0.043 g) and N,N-dimethylaminopyridine (0.005 g) in dry N,N-dimethylformamide (4 ml) were stirred at ambient temperature for 8 hours. The reaction mixture was poured onto a solution of brine/ethyl acetate. The aqueous layer was separated and washed thrice with ethyl acetate. The organic phases were combined, dried over sodium sulphate, filtered and evaporated under reduced pressure. The resulting crude mixture was purified by chromatography on silica gel (eluant: heptane/ethyl acetate: 1/1) to give 2-(3-bromo-quinolin-6-yloxy)-N-tert-butyl-2-methoxy acetamide (Compound No. 12 of Table 155) as a yellow oil (0.046 g).

¹H NMR (CDCl₃) δ ppm: 8.80 (1H, d); 8.22 (1H, d); 8.03 (1H, d); 7.52 (1H, d×d); 7.38 (1H, d); 6.45 (1H, br s); 5.36 (1H, s); 3.52 (3H, s); 1.38 (9H, s).

The following amides were prepared using a similar procedure.

Compound No. 12 of Table 99: N-tert-Butyl-2-(3,8-dibromo-quinolin-6-yloxy)-2-methoxy-acetamide using (3,8-dibromo-quinolin-6-yloxy)-methoxy acetic acid and N-tert-butylamine. ¹H NMR (CDCl₃) δ ppm: 8.89 (1H, d); 8.23 (1H, d); 7.88 (1H, d); 7.49 (1H, d); 6.45 (1H, br s); 5.35 (1H, s); 3.52 (3H, s); 1.39 (9H, s).

Compound No. 47 of Table 155: 2-(3-Bromo-quinolin-6-yloxy)-2-methoxy-N-(2-methoxy-1,1-dimethyl-ethyl)-acetamide using (3-bromo-quinolin-6-yloxy)-methoxy-acetic acid and 2-methoxy-1,1-dimethyl-ethylamine, ¹H NMR (CDCl₃) □ ppm: 8.88 (1H, d); 8.21 (1H, d); 8.02 (1H, d); 7.51 (1H, d×d); 7.38 (1H, d); 6.8 (1H, br s); 5.38 (1H, s); 3.51 (3H, s); 3.37 (2H, m); 3.35 (3H, s); 1.39 (3H, s); 1.37 (3H, s).

Compound No. 52 of Table 155: 2-(3-Bromo-quinolin-6-yloxy)-N-(1-cyano-2-methoxy-1-methyl-ethyl)-2-methoxy-acetamide using (3-bromo-quinolin-6-yloxy)-methoxy-acetic acid and 2-cyano-1-methoxy-prop-2-ylamine, ¹H NMR (CDCl₃) δ ppm: diastereoisomeric mixture (1/1); a) 8.81 (1H, d); 8.24 (1H, d); 8.02 (1H, d); 7.51 (1H, d×m); 7.41 (1H, d); 7.11 (1H, br s); 5.48 (1H, s); 3.75-3.6 (2H, m); 3.55 (3H, s); 3.51 (3H, s); 1.77 (3H, s); b) 8.81 (1H, d); 8.24 (1H, d); 8.02 (1H, d); 7.51 (1H, d×m); 7.38 (1H, d); 7.03 (1H, br s); 5.48 (1H, s); 3.75-3.6 (2H, m); 3.55 (3H, s); 3.44 (3H, s); 1.72 (3H, s).

Compound No. 12 of Table 156: 2-(3-Bromo-quinolin-6-yloxy)-N-tert-butyl-2-ethoxy-acetamide using (3-bromo-quinolin-6-yloxy)-ethoxy-acetic acid and N-tert-butylamine. ¹H NMR (CDCl₃) δ ppm: 8.79 (1H, d); 8.22 (1H, d); 8.01 (1H, d); 7.50 (1H, d×d); 7.38 (1H, d); 6.48 (1H, br s); 5.42 (1H, s); 3.85 (1H, m); 3.68 (1H, m); 1.39 (9H, s); 1.28 (3H, t).

Compound No. 47 of Table 156: 2-(3-Bromo-quinolin-6-yloxy)-2-ethoxy-N-(2-methoxy-1,1-dimethyl-ethyl)-acetamide using (3-bromo-quinolin-6-yloxy)-ethoxy-acetic acid and 2-methoxy-1,1-dimethyl-ethylamine.

¹H NMR (CDCl₃) δ ppm: 8.79 (1H, d); 8.22 (1H, d); 8.01 (1H, d); 7.50 (1H, d×d); 7.37 (1H, d); 6.8 (1H, br s); 5.44 (1H, s); 3.85 (1H, m); 3.69 (1H, m); 3.38 (2H, m); 3.35 (3H, s); 1.39 (3H, s); 1.37 (3H, s); 1.28 (3H, t).

Compound No. 52 of Table 156: 2-(3-Bromo-quinolin-6-yloxy)-N-(1-cyano-2-methoxy-1-methyl-ethyl)-2-ethoxy-acetamide using (3-bromo-quinolin-6-yloxy)-ethoxy-acetic acid and 2-cyano-1-methoxy-prop-2-ylamine.

¹H NMR (CDCl₃) δ ppm: diastereoisomeric mixture (1/1); a) 8.80 (1H, d); 8.24 (1H, d); 8.02 (1H, d); 7.49 (1H, d×m); 7.40 (1H, d); 7.13 (1H, br s); 5.52 (1H, s); 3.88 (1H, m); 3.72 (1H, m); 3.73-3.6 (2H, m); 3.51 (3H, s); 1.78 (3H, s); 1.29 (3H, t); b) 8.80 (1H, d); 8.24 (1H, d); 8.02 (1H, d); 7.49 (1H, d×m); 7.37 (1H, d); 7.03 (1H, br s); 5.52 (1H, s); 3.88 (1H, m); 3.72 (1H, m); 3.73-3.6 (2H, m); 3.45 (3H, s); 1.73 (3H, s); 1.29 (3H, t).

### EXAMPLE 3

This Example illustrates the preparation of (3-bromo-quinolin-6-yloxy)methoxyacetic acid methyl ester according to Scheme 3 for m=1.

### Step 1: Preparation of bromo-methoxy-acetic acid methyl ester

A suspension of methoxyacetic acid methyl ester (0.400 g), N-bromosuccinimide (0.752 g) and azo-isobutyronitrile (0.126 g) in carbon tetrachloride (6 ml) was heated at reflux for 20 hours. The reaction mixture was cooled down to ambient temperature, filtered off and concentrated in vacuum to give bromomethoxy-acetic acid methyl ester (0.94 g) as a yellow liquid. The crude mixture was used in the next step without further purification.

¹H NMR (CDCl₃) δ ppm: 6.01 (1H, s); 3.88 (3H, s); 3.60 (3H, s).

### Step 2: Preparation of (3-Bromo-quinolin-6-yloxy)methoxyacetic acid methyl ester

To a solution of potassium tert-butoxide (0.308 g) in tert-butyl alcohol (5 ml) at ambient temperature was added a solution of 3-bromo-quinolin-6-ol (0.454 g) in tert-butyl alcohol (1 ml). The resulting solution was stirred at R.T. for 15 minutes. Then, a solution of bromomethoxy-acetic acid methyl ester (0.500 g) in tert-butyl alcohol (1 ml), along with a catalytic amount of potassium iodide, were added. The reaction mixture was stirred at ambient temperature for 3 hours and then poured onto water (25 ml)/chloroform (25 ml). The aqueous layer was separated and washed thrice with chloroform. The organic phases were combined, washed with brine, dried over sodium sulphate, filtered and evaporated under reduced pressure. The resulting crude mixture was purified by reverse phase HPLC (eluant: acetonitrile/water) to give (3-bromo-quinolin-6-yloxy)methoxyacetic acid methyl ester (0.177 g) as a yellow oil [¹H NMR (CDCl₃) δ ppm: 8.80 (1H, d); 8.22 (1H, d); 8.02 (1H, d); 7.51 (1H, d×d); 7.30 (1H, d); 5.63 (1H, s); 3.87 (3H, s); 3.58 (3H, s)] which can provide compounds of the general formula (1) as shown in Scheme 3 in a similar manner as described in Example 2, Steps 4 and 5.

### EXAMPLE 4

This Example illustrates the preparation of N-(3-fluoro-1,1-dimethyl-propyl)amine hydrochloride according to Scheme 9.

### Step 1: Preparation of (3-Fluoro-1,1-dimethyl-propyl)-carbamic acid tert-butyl ester

4,4-dimethyl-2,2-dioxo-(1,2,3)oxathiazinane (23.9 g) (prepared according the procedure described in J. Am. Chem. Soc. 2001, 123, 6935-6936) was dissolved in dry CH₃CN (500 ml) under a nitrogen atmosphere. Ethyl diisopropylamine (Hünig's base) (52.1 ml) and dimethylaminopyridine (DMAP) (1.8 g) were added followed by (tBuOCO)₂O (56.9 g). The mixture was stirred at 50° C. during 3 hours then poured into water and diluted with ethyl acetate. The organic phase was separated, washed with brine, dried over sodium sulphate, filtered and evaporated to give the crude product which was purified by flash chromatography (cyclohexane:ethyl acetate, 2:1) to give 24.1 g pure 4,4-dimethyl-2,2-dioxo-(1,2,3)oxathiazinane-3-carboxylic acid tertbutyl ester as a yellow solid. ¹H NMR (CDCl₃) δ ppm: 4.55 (1H, t); 2.30 (1H, t); 1.65 (6H, s); 1.52 (9H, s)

To a solution of TBAF (6.8 ml of a 1M solution in THF) in CH₃CN at room temperature, under a nitrogen atmosphere, was added 4,4-dimethyl-2,2-dioxo-(1,2,3)oxathiazinane-3-carboxylic acid tertbutyl ester (1.5 g). The mixture was stirred overnight then diluted with ether (20 ml) and quenched with 1M HCl (7 ml). After separation, the organic phase was washed with brine, dried over sodium sulphate anhydrous, filtered and concentrated. The crude product was purified by flash chromatography (cyclohexane:ethyl acetate, 4:1) to give 1.08 g of pure (3-fluoro-1,1-dimethyl-propyl)-carbamic acid tert-butyl ester as a light yellow oil. ¹H NMR (CDCl₃) δ ppm: 4.65 (1H, t); 4.55 (1H, br s); 4.50 (1H, t); 2.15-2.05 (2H, m); 1.45 (9H, s); 1.30 (6H, s)

### Step 2: Preparation of N-(3-Fluoro-1,1-dimethyl-propyl)amine hydrochloride

A 2M solution of HCl in ethyl ether (25 ml) was added to (3-fluoro-1,1-dimethyl-propyl)-carbamic acid tert-butyl ester (1.08 g) at room temperature, under a nitrogen atmosphere. The mixture was stirred overnight, then a 2M solution of HCl in ethyl ether (12 ml) was added again. After stirring during 2 days the mixture was concentrated to give a N-(3-fluoro-1,1-dimethyl-propyl)amine hydrochloride (0.77 g) as a solid which was pure to be used for the next steps. ¹H NMR (DMSO-d₆) δ ppm: 4.62 (1H, t); 4.50 (1H, t); 3.70-3.45 (br s); 2.00-1.85 (2H, m); 1.20 (6H, s).

### EXAMPLE 5

This Example illustrates the preparation of 2-(3-iodo-quinolin-6-yloxy)-N-(3-fluoro-1,1-dimethyl-propyl) butyramide (Compound No. 275 of Table 158) according to Scheme 3 wherein m=0.

### Step 1: Preparation of 2-(3-Iodo-quinolin-6-yloxy)-butyric acid ethyl ester

### Stage 1: Preparation of 3-iodo-6-hydroxyquinoline

To a stirred mixture of 3-bromo-6-hydroxyquinoline (preparation described in Liebigs Ann Chem 1966, 98-106), (3.0 g), sodium iodide (4.0 g) and copper iodide (0.25 g) in dioxane (19.5 ml) was added N,N,N′,N′-tetramethyl-ethane-1,2-diamine (0.24 g) in a sealed tube. The mixture was stirred at 120° C. for 14 h and upon cooling was treated with aqueous ammonia followed by aqueous hydrochloric acid. Extraction with ethyl acetate, drying of the organic phase over magnesium sulphate, filtration and evaporation under reduced pressure gave the required product (M⁺ 272) as a light brown coloured powder that was used as such in the next step.

Stage 2: 3-Iodo-quinolin-6-ol (1.0 g) from Step 1, Stage 1 was dissolved in dry DMF (20 ml). 2-Bromo-butyric acid ethyl ester (940 mg) and dry potassium carbonate (1.5 g) were added to the mixture at R.T. The resulting suspension was stirred at 50° C. for 3 hour. The reaction mixture was poured into brine and extracted 3 times with ethyl acetate. The organic layers were combined, washed with brine, dried over sodium sulphate, filtered and evaporated to give 0.86 g og 2-(3-iodo-quinolin-6-yloxy)-butyric acid ethyl ester which after flash chromatography (cyclohexane:ethyl acetate, 6:1). ¹H NMR (CDCl₃) δ ppm: 8.86 (1H, d); 8.36 (1H, d); 7.95 (1H, d); 7.41 (1H, dd); 6.85 (1H, d); 4.65 (1H, t); 4.22 (2H, q); 2.05 (2H, m); 1.25 (3H, t); 1.10 (3H, t).

### Step 2: Preparation of 2-(3-Iodo-quinolin-6-yloxy)-butyric acid

To a solution of 2-(3-iodo-quinolin-6-yloxy)-butyric acid ethyl ester (860 mg) in tetrahydrofurane (9 ml) at room temperature is added a 0.5M aqueous solution of NaOH (5.4 ml). The reaction mixture is stirred 4 h at room temperature. Ethyl acetate was added and the two phases were separated. The aqueous phase was acidified with 1M HCl (until pH 2-3) then extracted twice with ethyl acetate. The organic phases were combined, washed with brine, dried over sodium sulphate, filtered and evaporated to give 0.77 g of crude 2-(3-iodo-quinolin-6-yloxy)-butyric acid which was used in the next step without further purification. ¹H NMR (DMSO-d₆) δ ppm: 8.85 (1H, d); 8.20 (1H, d); 7.90 (1H, d); 7.45 (1H, dd); 7.20 (1H, d); 4.85 (1H, t); 2.00 (2H, m); 1.05 (3H, t).

### Step 3: Preparation of 2-(3-Iodo-quinolin-6-yloxy)-N-(3-Fluoro-1,1-dimethyl-propyl) butyramide

2-(3-Iodo-quinolin-6-yloxy)-butyric acid (100 mg), N-(3-fluoro-1,1-dimethyl-propyl) amine hydrochloride (44 mg) from Step 2, Example 4, 1-hydroxy-7-azabenzotriazole (HOAT) (42 mg), O-(Benzotriazol-1-yl)-N,N,N′,N′-tetramethyluronium tetrafluoroborate (TBTU) (100 mg) and triethylamine (0.2 ml) in dry CH₃CN (7 ml) were stirred at ambient temperature overnight. The reaction mixture was quenched with a saturated solution of NaHCO₃ and extracted with ethyl acetate. The organic phase was washed with brine and dried over sodium sulphate, filtered and evaporated. The crude product was purified by flash chromatography (cyclohexane:ethyl acetate, 3:1) to give 0.12 g of 2-(3-iodo-quinolin-6-yloxy)-N-(3-fluoro-1,1-dimethyl-propyl)-butyramide as a yellow oil.

Compound 275 of Table 158: ¹H NMR (CDCl₃) δ ppm: 8.90 (1H, d); 8.40 (1H, d); 7.96 (1H, d); 7.40 (1H, dd); 6.95 (1H, d); 6.40 (1H, br s); 4.60-4.35 (3H, m); 2.20-1.95 (4H, m); 1.35 (3H, s); 1.32 (3H, s); 1.05 (3H, t).

### EXAMPLE 6

This Example illustrates the preparation of 2-(3-bromo-8-methyl-quinolin-6-yloxy)-N-tert-butyl-butyramide (Compound No. 12 of Table 90) according to Scheme 3 wherein m=0.

### Stage 1: Preparation of 3-bromo-6-hydroxy-8-methylquinoline

6-Amino-3-bromo-8-methylquinoline (12 g) (preparation described in Journal of the American Chemical Society (1955), pages 4175-4176) was suspended in a mixture of water (5 ml) and phosphoric acid (60 ml) and heated in a sealed glass tube to 180° C. for 3 days. The mixture was cooled to ambient temperature, diluted with water then taken to pH 3-4 with aqueous (2M) sodium hydroxide. The precipitate formed was filtered from solution, washed with cold water and sucked to dryness to give 3-bromo-6-hydroxy-8-methylquinoline as a grey solid.

¹H NMR (d6-DMSO) δ ppm: 2.56 (3H, s); 3.50 (1H, bs); 6.91 (1H, d); 7.15 (1H, d); 8.38 (1h, d); 8.61 (1H, d).

The procedures of example 1, steps 1-3 is repeated using 3-bromo-6-hydroxy-8-methylquinoline from stage 1 above as a staring material to provide 2-(3-bromo-8-methyl-quinolin-6-yloxy)-N-tert-butyl-butyramide (Compound No. 12 of Table 90):

¹H NMR (CDCl₃) δ ppm: 8.79 (1H, d); 8.14 (1H, d); 7.27 (1H, d); 6.83 (1H, d); 6.10 (1H, br s); 4.49 (1H, dd); 2.75 (3H, s); 2.06-1.93 (2H, m), 1.30 (9H, s), 1.05 (3H, t).

The following amides were prepared using a similar procedure.

Compound No. 47 of Table 90: 2-(3-Bromo-8-methyl-quinolin-6-yloxy)-N-(2-methoxy-1,1-dimethyl-ethyl)-butyramide using 2-(3-bromo-8-methyl-quinolin-6-yloxy)-butyric acid and 1-methoxy-2-methylprop-2-ylamine:

¹H NMR (CDCl₃) δ ppm: 8.79 (1H, d); 8.15 (1H, d); 7.27 (1H, d); 6.84 (1H, d); 6.42 (1H, br s); 4.50 (1H, dd); 3.29 (2H, dd), 3.27 (3H, s), 2.76 (3H, s), 2.08-1.94 (2H, m), 1.33 (3H, s), 1.28 (3H, s), 1.06 (3H, t).

Compound No. 52 of Table 90: 2-(3-Bromo-8-methyl-quinolin-6-yloxy)-N-(cyano-methoxymethyl-methyl-methyl)-butyramide using 2-(3-bromo-8-methyl-quinolin-6-yloxy)-butyric acid and 2-cyano-1-methoxy-prop-2-ylamine:

¹H NMR (CDCl₃) δ ppm: diastereoisomeric mixture; 8.78 (1H, m); 8.18-8.14 (1H, d); 7.27 (1H, m); 6.88-6.79 (2H, m), 4.70-4.62 (1H, m), 3.69-3.52 (2H, m), {3.43 (s) & 3.28 (s) 3H}, 2.74 (3H, s), 2.13-2.00 (2H, m), {1.72 (s) & 1.64 (s), 3H}, 1.10-1.06 (3H, m).

### EXAMPLE 7

This Example illustrates the preparation of 1-methoxy-3-methylbut-3-ylamine hydrochloride according to Scheme 9.

### Stage 1: Preparation of 1-methoxy-3-methylbut-3-ylamine hydrochloride

To a stirred suspension of sodium hydride (0.30 g, 80% dispersion in mineral oil) in dry N,N-dimethylformamide (2 ml) under an atmosphere of nitrogen at ambient temperature was added dropwise a solution of 1-hydroxy-3-methylbut-3-ylamine (0.52 g) in N,N-dimethylformamide (5 ml). The mixture was stirred for 3 hours, methyl iodide (0.74 g) in N,N-dimethylformamide (5 ml) added over 5 minutes then stirred for another 2.25 hours and stored for 18 hours at ambient temperature. The solution was diluted with water, extracted into ethyl acetate (three times) and the extracts combined then extracted with dilute hydrochloric acid. The aqueous acidic extract was evaporated under reduced pressure and co-distilled with toluene to remove residual water to give 1-methoxy-3-methylbut-3-ylamine hydrochloride as a yellow gum.

¹H NMR (CDCl₃) δ ppm: 1.54 (6H, s); 1.96-2.00 (2H, t); 3.48 (3H, s); 3.62-3.66 (2H, t).

In a similar procedure, 1-hydroxy-3-methylbut-3-ylamine was reacted with ethyl iodide to give 1-ethoxy-3-methylbut-3-ylamine hydrochloride.

¹H NMR (CDCl₃) δ ppm: 1.20-1.24 (3H, t); 1.54 (6H, s); 1.96-2.00 (2H, t); 3.50 (2H, q); 3.66-3.70 (2H, t).

In a similar procedure, 1-hydroxy-2-methylprop-2-ylamine was reacted with methyl iodide to give 1-methoxy-2-methylprop-2-ylamine hydrochloride.

¹H NMR (CDCl₃) δ ppm: 1.47 (6H, s); 3.43 (3H, s); 3.44 (2H, s); 8.24 (3H bs).

In a similar procedure, 1-hydroxy-2-methylprop-2-ylamine was reacted with 4-fluorobenzyl bromide to give 1-(4-fluorobenzyloxy)-2-methylprop-2-ylamine hydrochloride.

¹H NMR (CDCl₃) δ ppm: 1.41 (6H, s); 3.46 (2H, s); 4.53 (2H, s); 7.00-7.04 (2H, m); 7.32-7.36 (2H, m); 8.30 (3H bs).

### EXAMPLE 8

### Table 161

The characterized compounds in Table 161 are of the general formula (I) where Q⁴, Q⁵ and Q⁶ are hydrogen, L is O, R² is hydrogen, and Q¹, Q², Q³, m, R¹ and R³ have the values given in the table.

| Cpd. | | | | | | | Physical/spectral |
|---|---|---|---|---|---|---|---|
| No. | Q¹ | Q² | Q³ | m | R¹ | R³ | data |
| 1 | Br | H | H | 1 | Me | C(CH₃)₃ | m.p. = 104-105° C. |
| 2 | Br | H | H | 1 | Et | C(CH₃)₃ | ¹H NMR (CDCl₃) |
| | | | | | | | [symbol] ppm: 8.79 (1H, |
| | | | | | | | d); 8.22 (1H, d); |
| | | | | | | | 8.00 (1H, d); |
| | | | | | | | 7.50 (1H, dd); 7.48 (1H, |
| | | | | | | | d); 6.48 (1H, s br); |
| | | | | | | | 5.41 (1H, s); |
| | | | | | | | 3.90 to 3.53 (2H, m); |
| | | | | | | | 1.38 (9H, s); |
| | | | | | | | 1.28 (3H, t). |
| 3 | Br | H | H | 1 | Me | C(CH₃)₂CH₂OCH₃ | ¹H NMR (CDCl₃) |
| | | | | | | | [symbol] ppm: 8.79 (1H, |
| | | | | | | | d); 8.22 (1H, d); |
| | | | | | | | 8.00 (1H, d); |
| | | | | | | | 7.51 (1H, dd); 7.48 (1H, |
| | | | | | | | d); 6.79 (1H, s br); |
| | | | | | | | 5.47 (1H, s); |
| | | | | | | | 3.50 (3H, s); 3.40 to |
| | | | | | | | 3.34 (5H, m); |
| | | | | | | | 1.40 (6H, d). |
| 4 | Br | H | H | 1 | Me | C(CH₃)(CN)CH₂OCH₃ | ¹H NMR (CDCl₃) |
| | | | | | | | [symbol] ppm: 8.80 (1H, |
| | | | | | | | d); 8.23 (1H. d); |
| | | | | | | | 8.02 (1H, d); |
| | | | | | | | 7.51 (1H, m); 7.39 (1H, |
| | | | | | | | dd); 7.11, |
| | | | | | | | 7.03 (1H, s br, 2 |
| | | | | | | | isomers); 5.49, |
| | | | | | | | 5.47 (1H, s, |
| | | | | | | | isomers A + B); |
| | | | | | | | 3.74 to 3.60 (2H, |
| | | | | | | | m, isomers A + B); |
| | | | | | | | 3.56 (3H, s); 3.51, |
| | | | | | | | 3.43 (3H, s, |
| | | | | | | | isomers A + B); |
| | | | | | | | 1.77, 1.73 (3H, s, |
| | | | | | | | isomers A + B). |
| 5 | Br | Br | H | 1 | Me | C(CH₃)₃ | m.p. = 154-157° C. |
| 6 | Br | H | H | 1 | Et | C(CH₃)₂CH₂OCH₃ | ¹H NMR (CDCl₃) |
| | | | | | | | [symbol] ppm: 8.79 (1H, |
| | | | | | | | d); 8.22 (1H, d); |
| | | | | | | | 8.02 (1H, d); |
| | | | | | | | 7.50 (1H, dd); 7.48 (1H, |
| | | | | | | | d); 6.81 (1H, s br); |
| | | | | | | | 5.43 (1H, s); |
| | | | | | | | 3.91 to 3.62 (2H, m); |
| | | | | | | | 3.39 (2H, s); |
| | | | | | | | 3.37 (3H, s); 1.38 (6H, |
| | | | | | | | d); 1.28 (3H, t). |
| 7 | Br | H | H | 1 | Et | C(CH₃)(CN)CH₂OCH₃ | ¹H NMR (CDCl₃) |
| | | | | | | | [symbol] ppm: 8.80 (1H, |
| | | | | | | | d); 8.22 (1H, d); |
| | | | | | | | 8.02 (1H, d); |
| | | | | | | | 7.48 (1H, m); 7.38 (1H, |
| | | | | | | | dd); 7.13, |
| | | | | | | | 7.03 (1H, s br, isomers |
| | | | | | | | A + B); 5.53, |
| | | | | | | | 5.51 (1H, s, isomers |
| | | | | | | | A + B); 3.92 to |
| | | | | | | | 3.83 (1H, m); 3.75 to |
| | | | | | | | 3.61 (3H, m); |
| | | | | | | | 3.50, 3.43 (3H, s, |
| | | | | | | | isomers A + B); |
| | | | | | | | 1.77, 1.72 (3H, s, |
| | | | | | | | isomers A + B); |
| | | | | | | | 1.38 (3H, m). |
| 8 | Br | Cl | H | 1 | Me | C(CH₃)₃ | m.p. = 130-134° C. |
| 9 | Br | Br | H | 1 | Et | C(CH₃)₃ | m.p. = 157-160° C. |
| 10 | Br | Br | H | 1 | Me | C(CH₃)(CN)CH₂OCH₃ | m.p. = 120-122° C. |
| 11 | Br | Br | H | 1 | Me | C(CH₃)₂CH₂OCH₃ | m.p. = 80-83° C. |
| 12 | Br | H | H | 1 | CH₂(cyclopropyl) | C(CH₃)₃ | ¹H NMR (CDCl₃) |
| | | | | | | | [symbol] ppm: 8.80 (1H, |
| | | | | | | | d); 8.22 (1H, d); |
| | | | | | | | 8.02 (1H, d); |
| | | | | | | | 7.50 (1H, dd); 7.38 (1H, |
| | | | | | | | d); 6.50 (1H, s br); |
| | | | | | | | 5.50 (1H, s); |
| | | | | | | | 3.65 to 3.55 (1H, m); |
| | | | | | | | 3.50 to 3.46 (1H, |
| | | | | | | | m); 1.40 (9H, s); |
| | | | | | | | 1.09 (1H, m); |
| | | | | | | | 0.53 (2H, d); 0.20 (2H, |
| | | | | | | | m). |
| 13 | Br | H | H | 1 | CH₂CHCH₂ | C(CH₃)₃ | ¹H NMR (CDCl₃) |
| | | | | | | | [symbol] ppm: 8.80 (1H, |
| | | | | | | | d); 8.22 (1H, d); |
| | | | | | | | 8.02 (1H, d); |
| | | | | | | | 7.51 (1H, dd); 7.49 (1H, |
| | | | | | | | d); 6.47 (1H, s br); |
| | | | | | | | 5.98 to 5.86 (1H, |
| | | | | | | | m); 5.46 (1H, s); |
| | | | | | | | 5.38 to 5.27 (2H, |
| | | | | | | | m); 4.33 (1H, dd); |
| | | | | | | | 4.18 (1H, dd); |
| | | | | | | | 1.39 (9H, s). |
| 14 | Br | H | H | 1 | CH₂CF₃ | C(CH₃)₃ | m.p. = 127-130° C. |
| 15 | Br | H | H | 1 | CH(CH3)₂ | C(CH₃)₃ | ¹H NMR (CDCl₃) |
| | | | | | | | [symbol] ppm: 8.80 (1H, |
| | | | | | | | d); 8.22 (1H, d); |
| | | | | | | | 8.00 (1H, d); |
| | | | | | | | 7.49 (1H, dd); 7.38 (1H, |
| | | | | | | | d); 6.49 (1H, s br); |
| | | | | | | | 5.45 (1H, s); |
| | | | | | | | 4.03 (1H, quint.); |
| | | | | | | | 1.37 (9H, s); 1.27 (3H, |
| | | | | | | | d); 1.20 (3H, d). |
| 16 | Br | H | H | 1 | (CH₂)₂OCH3 | C(CH₃)₃ | m.p. = 58-62° C. |
| 17 | Br | H | H | 1 | CH(CH2F)₂ | C(CH₃)₃ | m.p. = 124-128° C. |
| 18 | Br | H | H | 1 | CH₂CCH | C(CH₃)₃ | ¹H NMR (CDCl₃) |
| | | | | | | | [symbol] ppm: 8.80 (1H, |
| | | | | | | | d); 8.22 (1H, d); |
| | | | | | | | 8.01 (1H, d); |
| | | | | | | | 7.51 (1H, dd); 7.43 (1H, |
| | | | | | | | d); 6.43 (1H, s br); |
| | | | | | | | 5.62 (1H, s); |
| | | | | | | | 4.44 (2H, m); 2.47 (1H, |
| | | | | | | | s); 1.49 (9H, s). |
| 19 | Br | H | H | 1 | (CH₂)₂SCH3 | C(CH₃)₃ | ¹H NMR (CDCl₃) |
| | | | | | | | [symbol] ppm: 8.80 (1H, |
| | | | | | | | d); 8.22 (1H, d); |
| | | | | | | | 8.01 (1H, d); |
| | | | | | | | 7.51 (1H, dd); 7.39 (1H, |
| | | | | | | | d); 6.58 (1H, s br); |
| | | | | | | | 5.47 (1H, s); |
| | | | | | | | 4.00 to 3.93 (1H, m); |
| | | | | | | | 3.81 to 3.64 (1H, |
| | | | | | | | m); 2.73 (2H, m); |
| | | | | | | | 2.11 (3H, s); |
| | | | | | | | 1.39 (9H, s). |
| 20 | Br | H | H | 1 | CH₂CCCH3 | C(CH₃)₃ | ¹H NMR (CDCl₃) |
| | | | | | | | [symbol] ppm: 8.80 (1H, |
| | | | | | | | d); 8.22 (1H, d); |
| | | | | | | | 8.01 (1H, d); |
| | | | | | | | 7.52 (1H, dd); 7.42 (1H, |
| | | | | | | | d); 6.48 (1H, s br); |
| | | | | | | | 5.62 (1H, s); |
| | | | | | | | 4.47 to 4.34 (2H, m); |
| | | | | | | | 1.79 (3H, m); |
| | | | | | | | 1.39 (9H, s). |
| 21 | Br | H | H | 1 | CH₂CHF₂ | C(CH₃)₃ | m.p. = 102-1106° C. |
| 22 | Br | Me | H | 1 | Me | C(CH₃)₃ | m.p. = 110-113° C. |
| 23 | Br | Me | H | 1 | Me | C(CH₃)₂CH₂OCH₃ | m.p. = 55-60° C. |
| 24 | Br | Me | H | 1 | Me | C(CH₃)(CN)CH₂OCH₃ | m.p. = 95-99° C. |
| 25 | I | H | H | 1 | Me | C(CH₃)₃ | m.p. = 125-128° C. |
| 26 | I | Me | H | 1 | Me | C(CH₃)₃ | m.p. = 132-136° C. |
| 27 | Br | H | F | 1 | Me | C(CH₃)₃ | m.p. = 120-123° C. |
| 28 | Br | H | H | 1 | Me | C(CH₃)₂CH₂OH | ¹H NMR (CDCl₃) |
| | | | | | | | [symbol] ppm: 8.82 (1H, |
| | | | | | | | d); 8.22 (1H, d); |
| | | | | | | | 8.02 (1H, d), |
| | | | | | | | 7.50 (1H, dd); 7.38 (1H, |
| | | | | | | | d); 6.70 (1H, s br); |
| | | | | | | | 5.42 (1H, s); |
| | | | | | | | 3.89 (1H, t); 3.70 to |
| | | | | | | | 3.59 (2H, m); |
| | | | | | | | 3.52 (3H, s); 1.33 (6H, |
| | | | | | | | d). |
| 29 | Br | Me | H | 1 | Me | C(CH₃)₂CH₂OH | ¹H NMR (CDCl₃) |
| | | | | | | | [symbol] ppm: 8.82 (1H, |
| | | | | | | | d); 8.20 (1H, d); |
| | | | | | | | 7.35 (1H, m); |
| | | | | | | | 7.20 (1H, d); 6.70 (1H, s |
| | | | | | | | br); 5.41 (1H, s); |
| | | | | | | | 3.92 (1H, t); |
| | | | | | | | 3.70 to 3.60 (2H, m); |
| | | | | | | | 3.52 (3H, s); |
| | | | | | | | 2.78 (3H, s); 1.33 (6H, |
| | | | | | | | d). |
| 30 | I | H | H | 1 | Me | C(CH₃)₂CH₂OH | ¹H NMR (CDCl₃) |
| | | | | | | | [symbol] ppm: 8.82 (1H, |
| | | | | | | | d); 8.47 (1H, d); |
| | | | | | | | 8.01 (1H, d); |
| | | | | | | | 7.51 (1H, dd); 7.35 (1H, |
| | | | | | | | d); 6.70 (1H, s br); |
| | | | | | | | 5.41 (1H, s); |
| | | | | | | | 3.90 (1H, s); 3.62 (2H, |
| | | | | | | | s); 3.51 (3H, s); |
| | | | | | | | 1.33 (6H, d). |
| 31 | I | Me | H | 1 | Me | C(CH₃)₂CH₂OH | m.p. = 105-107° C. |
| 32 | Br | H | H | 1 | Me | C(CH₃)₂CH(O) | ¹H NMR (CDCl₃) |
| | | | | | | | [symbol] ppm: 9.39 (1H, |
| | | | | | | | s); 8.81 (1H, d); |
| | | | | | | | 8.23 (1H, d); |
| | | | | | | | 8.02 (1H, d); 7.52 (1H, |
| | | | | | | | dd); 7.39 (1H, d); |
| | | | | | | | 7.22 (1H, s br); |
| | | | | | | | 5.47 (1H, s); |
| | | | | | | | 3.54 (3H, s); 1.46 (6H, |
| | | | | | | | d). |
| 33 | Br | Me | H | 1 | Me | C(CH₃)₂CH(O) | m.p. = 148-150° C. |
| 34 | I | H | H | 1 | Me | C(CH₃)₂CH(O) | ¹H NMR (CDCl₃) |
| | | | | | | | [symbol] ppm: 9.39 (1H, |
| | | | | | | | s); 8.93 (1H, d); |
| | | | | | | | 8.46 (1H, d); |
| | | | | | | | 8.01 (1H, d); 7.52 (1H, |
| | | | | | | | dd); 7.33 (1H, d); |
| | | | | | | | 7.22 (1H, s br); |
| | | | | | | | 5.47 (1H, s); |
| | | | | | | | 3.54 (3H, s); 1.46 (6H, |
| | | | | | | | d). |
| 35 | I | Me | H | 1 | Me | C(CH₃)₂CH(O) | m.p. = 174-175° C. |
| 36 | H | H | H | 0 | Et | C(CH₃)(CN)CH₂OCH₃ | m.p. = 134-136° C. |
| 37 | H | H | H | 0 | Et | C(CH₃)CH(C(O)CH3)OH | m.p. = 130-132° C. |
| 38 | Br | H | H | 0 | CH(Me)₂ | C(CH₃)₃ | m.p. = 123-125° C. |
| 39 | Br | H | H | 0 | CH(Me)₂ | C(CH₃)(CN)CH₂OCH₃ | m.p. = 151-157° C. |
| 40 | Br | H | H | 0 | cyclopropyl | C(CH₃)₃ | m.p. = 165-167° C. |
| 41 | Br | H | H | 0 | cyclopropyl | C(CH₃)(CN)CH₂OCH₃ | m.p. = 119-121° C. |
| 42 | Br | H | H | 0 | cyclopropyl | C(CH₃)₂CH₂OCH₃ | m.p. = 139-141° C. |
| 43 | Br | H | H | 0 | Et | C(CH₃)₃ | ¹H NMR (CDCl₃) |
| | | | | | | | [symbol] ppm: 8.69 (1H, |
| | | | | | | | sm); 8.10 (1H, |
| | | | | | | | sm); 7.92 (1H, d); |
| | | | | | | | 7.32 (1H, dm); |
| | | | | | | | 6.92 (sm); 6.0 (1H, |
| | | | | | | | br s), 4.43 (1H, |
| | | | | | | | m); 1.95 (2H, m); |
| | | | | | | | 1.22 (9H, s); |
| | | | | | | | 0.96 (3H, t). |
| 44 | Br | H | H | 0 | Et | C(CH₃)₂CH₂OCH₃ | ¹H NMR (CDCl₃) |
| | | | | | | | [symbol] ppm: 8.75 (1H, |
| | | | | | | | sm); 8.16 (1H, |
| | | | | | | | sm); 7.98 (1H, d); |
| | | | | | | | 7.38 (1H, dm); |
| | | | | | | | 7.00 (sm); 6.4 (1H, |
| | | | | | | | br s); 4.51 (1H, |
| | | | | | | | m); 3.33-3.20 (2H, |
| | | | | | | | m); 3.23 (3H, s); |
| | | | | | | | 2.0 (2H, m); |
| | | | | | | | 1.30 (3H, s); 1.23 (3H, |
| | | | | | | | s); 1.05 (3H, t). |
| 45 | Br | H | H | 0 | Et | C(CH₃)(CN)CH₂OCH₃ | ¹H NMR (CDCl₃) |
| | | | | | | | [symbol] ppm: |
| | | | | | | | 8.72 (isomer A, 1H, |
| | | | | | | | sm); 8.68 (isomer |
| | | | | | | | B, 1H, sm); |
| | | | | | | | 8.18 (isomer A, 1H, |
| | | | | | | | sm); 8.10 (isomer |
| | | | | | | | B, 1H, sm); |
| | | | | | | | 7.98 (isomer A, 1H, d); |
| | | | | | | | 7.92 (isomer B, |
| | | | | | | | 1H, d); |
| | | | | | | | 7.42 (isomer B, 1H, |
| | | | | | | | dm); 7.38 (isomer |
| | | | | | | | A, 1H, dm); |
| | | | | | | | 7.0 (isomer A, 1H, |
| | | | | | | | sm); 6.95 (isomer |
| | | | | | | | B, 1H, sm); |
| | | | | | | | 6.90 + 6.85 (isomer |
| | | | | | | | A + B, 1H, 2 br s); |
| | | | | | | | 4.7-4.6 (isomer |
| | | | | | | | A + B, 1H, m); |
| | | | | | | | 3.75-3.50 (isomer A + B, |
| | | | | | | | 2H, m); 3.40 and |
| | | | | | | | 3.25 (isomers |
| | | | | | | | A + B, 3H, 2s); |
| | | | | | | | 2.15-1.95 (isomers |
| | | | | | | | A + B, 2H, m); |
| | | | | | | | 1.70 (isomer A, 3H, s); |
| | | | | | | | 1.60 (isomer B, |
| | | | | | | | 3H, s); |
| | | | | | | | 1.15 (isomer B, 3H, t); |
| | | | | | | | 1.04 (isomer A, |
| | | | | | | | 3H, t). |
| 46 | Br | Me | H | 0 | Et | C(CH₃)₃ | m.p. = 138-140° C. |
| 47 | Br | Me | H | 0 | Et | C(CH₃)(CN)CH₂OCH₃ | m.p. = 89-90° C. |
| 48 | I | H | H | 0 | Et | C(CH₃)₃ | m.p. = 125-126° C. |
| 49 | I | H | H | 0 | Et | C(CH₃)(CN)CH₂OCH₃ | m.p. = 100-101° C. |
| 50 | I | H | H | 0 | Et | C(CH₃)(CH₂)₂F | ¹H-NMR (CDCl₃) |
| | | | | | | | use spec. charac. #58 □ ppm: |
| | | | | | | | 8.90 (1H, d); |
| | | | | | | | 8.40 (1H, d); |
| | | | | | | | 7.96 (1H, d); |
| | | | | | | | 7.40 (1H, dd); |
| | | | | | | | 6.95 (1H, d); 6.40 (1H, br |
| | | | | | | | s); |
| | | | | | | | 4.60-4.35 (3H, m); |
| | | | | | | | 2.20-1.95 (4H, m); |
| | | | | | | | 1.35 (3H, s); |
| | | | | | | | 1.32 (3H, s); 1.05 (3H, t). |
| 51 | Br | Me | H | 0 | Et | C(CH₃)₂CH₂OCH₃ | m.p. = 117-120° C. |
| 52 | Br | Me | H | 0 | Et | C(CH₃)₂CH₂OH | m.p. = 99-100° C. |
| 53 | Br | Cl | H | 0 | Et | C(CH₃)₃ | m.p. = 126-127° C. |
| 54 | Br | Cl | H | 0 | Et | C(CH₃)₂CH₂OCH₃ | m.p. = 139-140° C. |
| 55 | Br | Cl | H | 0 | Et | C(CH₃)₂CH₂OH | m.p. = 125-127° C. |
| 56 | Br | Cl | H | 0 | Et | C(CH₃)(CN)CH₂OCH₃ | m.p. = 105-106° C. |
| 57 | Br | Me | H | 0 | Me | C(CH₃)₃ | m.p. = 155-157° C. |
| 58 | Br | Me | H | 0 | Me | C(CH₃)₂CH₂OCH₃ | m.p. = 104-106° C. |
| 59 | Br | Me | H | 0 | Me | C(CH₃)₂CH₂OH | m.p. = 98-99° C. |
| 60 | Br | Me | H | 0 | Me | C(CH₃)(CN)CH₂OCH₃ | m.p. = 136-138° C. |
| 61 | Br | Cl | H | 0 | Me | C(CH₃)₃ | m.p. = 148-150° C. |
| 62 | Br | Cl | H | 0 | Me | C(CH₃)₂CH₂OCH₃ | m.p. = 122-123° C. |
| 63 | Br | Cl | H | 0 | Me | C(CH₃)₂CH₂OH | m.p. = 102-104° C. |
| 64 | Br | Cl | H | 0 | Me | C(CH₃)(CN)CH₂OCH₃ | m.p. = 144-146° C. |
| 65 | Br | H | H | 0 | (CH₂)₂F | C(CH₃)₃ | m.p. = 126-128° C. |
| 66 | Br | H | H | 0 | (CH₂)₂OMe | C(CH₃)₃ | m.p. = 109-111° C. |
| 67 | I | Me | H | 0 | Et | C(CH₃)(CN)CH₂OCH₃ | ¹H-NMR (CDCl₃) |
| | | | | | | | □ ppm: 8.94 (1H, |
| | | | | | | | d, isomer A + B); |
| | | | | | | | 8.40 (1H, dd, |
| | | | | | | | isomer A + B); |
| | | | | | | | 7.27 (1H, m, isomer |
| | | | | | | | A + B); |
| | | | | | | | 6.85-6.75 (2H, m, isomer |
| | | | | | | | A + B); 4.67 (1H, m, |
| | | | | | | | isomer A + B); |
| | | | | | | | 3.72-3.46 (2H, m, |
| | | | | | | | isomer A + B); |
| | | | | | | | 3.45 (3H, s, isomer A); |
| | | | | | | | 3.29 (3H, s, |
| | | | | | | | isomer B); |
| | | | | | | | 2.74 (3H, s, isomer |
| | | | | | | | A + B); |
| | | | | | | | 2.10-2.00 (1H, m, 2H), |
| | | | | | | | 1.72 (3H, s, isomer A); |
| | | | | | | | 1.63 (3H, s, |
| | | | | | | | isomer B); |
| | | | | | | | 1.12-0.60 (3H, m, |
| | | | | | | | isomer A + B) |
| 68 | I | Me | H | 0 | Et | C(CH₃)₂(CH₂)₂SCH₃ | ¹H-NMR (CDCl₃) |
| | | | | | | | □ ppm: 8.95 (1H, |
| | | | | | | | d); 8.42 (1H, d); |
| | | | | | | | 7.30 (1H, d); |
| | | | | | | | 6.82 (1H, d); 6.13 (1H, |
| | | | | | | | s); 4.50 (1H, dd); |
| | | | | | | | 2.78 (3H, s); |
| | | | | | | | 2.31-2.22 (2H, m), |
| | | | | | | | 2.08-1.95 (4H, m); |
| | | | | | | | 1.98 (3H, s); |
| | | | | | | | 1.31 (3H, s); 1.29 (3H, |
| | | | | | | | s); 1.08 (3H, t) |
| 69 | I | Me | H | 0 | Et | C(CH₃)₂(CH₂)₂S(O)CH₃ | m.p. = 101-102° C. |
| 70 | I | Me | H | 0 | Et | C(CH₃)₂(CH₂)₂S(O)₂CH₃ | m.p. = 85-86° C. |
| 71 | I | H | H | 0 | Et | C(CH₃)₂CH₂CCH | ¹H-NMR (CDCl₃) |
| | | | | | | | □ ppm: 8.91 (1H, |
| | | | | | | | d); 8.41 (1H, d), |
| | | | | | | | 8.00 (1H, d); |
| | | | | | | | 7.42 (1H, dd); 6.99 (1H, |
| | | | | | | | d); 6.29 (1H, s); |
| | | | | | | | 4.52 (1H, dd); |
| | | | | | | | 2.7-2.55 (2H, m); |
| | | | | | | | 2.10-1.98 (2H, m); |
| | | | | | | | 1.72 (1H, t); |
| | | | | | | | 1.38 (3H, s); 1.09 (3H, |
| | | | | | | | t) |
| 72 | I | Me | H | 0 | Et | C(CH₃)₂CH₂CCH | ¹H-NMR (CDCl₃) |
| | | | | | | | □ ppm: 8.90 (1H, |
| | | | | | | | d); 8.37 (1H, d); |
| | | | | | | | 7.29 (1H, d); |
| | | | | | | | 6.30 (1H, d); 6.28 (1H, |
| | | | | | | | s), 4.50 (1H, dd); |
| | | | | | | | 2.72 (3H, s); |
| | | | | | | | 2.7-2.55 (2H, m); |
| | | | | | | | 2.01-1.97 (2H, m); |
| | | | | | | | 1.74 (1H, t); |
| | | | | | | | 1.37 (3H, s); 1.07 (3H, |
| | | | | | | | t) |
| 73 | I | Me | H | 0 | Et | C(CH₃)₃ | m.p. = 139-140° C. |
| 74 | I | Me | H | 0 | Et | C(CH₃)₂CH₂OCH₃ | m.p. = 136-138° C. |
| 75 | I | H | H | 0 | Et | C(CH₃)₂CH₂OH | m.p. = 88-90° C. |
| 76 | I | Me | H | 0 | Et | C(CH₃)₂CH(O) | m.p. = 153-154° C. |
| 77 | I | Me | H | 0 | Et | C(CH₃)₂CH₂OH | m.p. = 103-105° C. |
| 78 | I | H | H | 0 | Et | C(CH₃)₂CH(O) | m.p. = 148-150° C. |
| 79 | I | Me | H | 0 | Et | C(CH₃)₂(CH₂)₂S(O)(NH)CH₃ | m.p. = 58-59° C. |
| 80 | I | Me | H | 0 | Et | C(CH₃)₂(CH₂)₂S(O)(NCH₂CCH)CH₃ | m.p. = 51-52° C. |
| 81 | I | Me | H | 0 | Et | C(CH₃)₂(CH₂)₂S(O)(NC₆H₄pCl)CH₃ | m.p. = 58-59° C. |

### EXAMPLE 9

### Table 162

The characterized compounds in Table 162 are of the general formula (100) where Q⁴, Q⁵ and Q⁶ are hydrogen, L is O and, Q¹, Q², Q³, m, R⁵⁰⁰ have the values given in the table.

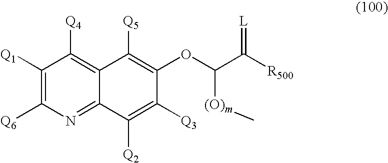

The following method was used for LC-MS analysis:

Method A: Method (Water Alliance 2795 LC) with the following HPLC gradient conditions (Solvent A: 0.1% of formic acid in water/acetonitrile (9:1) and Solvent B: 0.1% of formic acid in acetonitrile)

| Time (minutes) | A (%) | B (%) | Flow rate (ml/min) |
|---|---|---|---|
| 0 | 90 | 10 | 1.7 |
| 2.5 | 0 | 100 | 1.7 |
| 2.8 | 0 | 100 | 1.7 |
| 2.9 | 90 | 10 | 1.7 |

Type of column: Water atlantis dc18; Column length: 20 mm; Internal diameter of column: 3 mm; Particle Size: 3 micron; Temperature: 40° C.

| Cpd. | | | | | | | RT | |
|---|---|---|---|---|---|---|---|---|
| No. | Q1 | Q2 | Q3 | m | R¹ | R⁵⁰⁰ | (Min) | M + H |
| 1 | I | Me | H | 0 | Et | 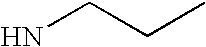 | 1.60 | 413.1 |
| 2 | I | Me | H | 0 | Et | 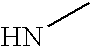 | 1.30 | 385.0 |
| 3 | I | Me | H | 0 | Et | 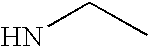 | 1.50 | 399.0 |
| 4 | I | Me | H | 0 | Et | 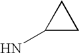 | 1.50 | 411.0 |
| 5 | I | Me | H | 0 | Et | 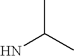 | 1.60 | 413.1 |
| 6 | I | Me | H | 0 | Et | 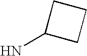 | 1.70 | 425.1 |
| 7 | I | Me | H | 0 | Et | 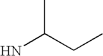 | 1.70 | 427.1 |
| 8 | I | Me | H | 0 | Et | 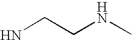 | 0.80 | 428.1 |
| 9 | I | Me | H | 0 | Et | 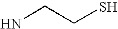 | 1.50 | 431.0 |
| 10 | I | Me | H | 0 | Et | 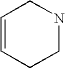 | 1.70 | 437.1 |
| 11 | I | Me | H | 0 | Et | 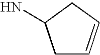 | 1.70 | 437.1 |
| 12 | I | Me | H | 0 | Et | 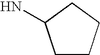 | 1.70 | 439.1 |
| 13 | I | Me | H | 0 | Et | 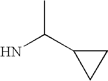 | 1.80 | 439.1 |
| 14 | I | Me | H | 0 | Et | 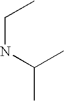 | 1.80 | 441.1 |
| 15 | I | Me | H | 0 | Et | 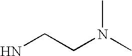 | 0.75 | 442.1 |
| 16 | I | Me | H | 0 | Et | 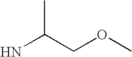 | 1.50 | 443.1 |
| 17 | I | Me | H | 0 | Et | 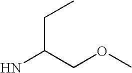 | 1.30 | 443.1 |
| 18 | I | Me | H | 0 | Et | 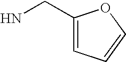 | 1.70 | 451.0 |
| 19 | I | Me | H | 0 | Et | 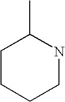 | 1.80 | 453.1 |
| 20 | I | Me | H | 0 | Et | 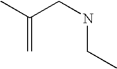 | 1.90 | 453.1 |
| 21 | I | Me | H | 0 | Et | 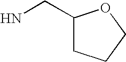 | 1.50 | 455.1 |
| 22 | I | Me | H | 0 | Et | 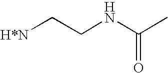 | 1.10 | 456.1 |
| 23 | I | Me | H | 0 | Et | 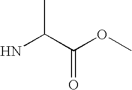 | 1.50 | 457.1 |
| 24 | I | Me | H | 0 | Et | 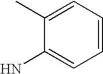 | 1.90 | 461.1 |
| 25 | I | Me | H | 0 | Et | 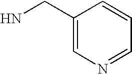 | 0.91 | 462.1 |
| 26 | I | Me | H | 0 | Et | 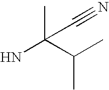 | 1.70 | 466.1 |
| 27 | I | Me | H | 0 | Et | 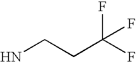 | 1.70 | 467.0 |
| 28 | I | Me | H | 0 | Et | 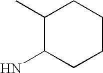 | 2.00 | 467.1 |
| 29 | I | Me | H | 0 | Et | 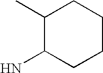 | 2.00 | 467.1 |
| 30 | I | Me | H | 0 | Et | 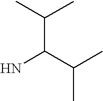 | 2.10 | 469.1 |
| 31 | I | Me | H | 0 | Et | 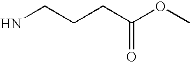 | 1.46 | 471.1 |
| 32 | I | Me | H | 0 | Et | 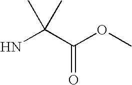 | 1.60 | 471.1 |
| 33 | I | Me | H | 0 | Et | 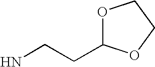 | 1.40 | 471.1 |
| 34 | I | Me | H | 0 | Et | 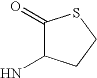 | 1.50 | 471.0 |
| 35 | I | Me | H | 0 | Et | 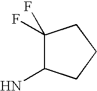 | 1.70 | 475.1 |
| 36 | I | Me | H | 0 | Et | 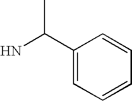 | 1.80 | 475.1 |
| 37 | I | Me | H | 0 | Et | 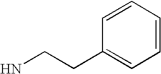 | 1.80 | 475.1 |
| 38 | I | Me | H | 0 | Et | 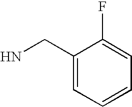 | 1.80 | 479.1 |
| 39 | I | Me | H | 0 | Et | 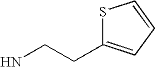 | 1.80 | 481.0 |
| 40 | I | Me | H | 0 | Et | 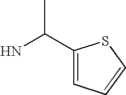 | 1.80 | 481.0 |
| 41 | I | Me | H | 0 | Et | 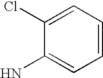 | 2.10 | 481.0 |
| 42 | I | Me | H | 0 | Et | 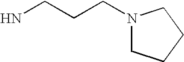 | 0.90 | 482.1 |
| 43 | I | Me | H | 0 | Et | 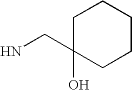 | 1.60 | 483.1 |
| 44 | I | Me | H | 0 | Et | 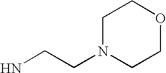 | 0.90 | 484.1 |
| 45 | I | Me | H | 0 | Et | 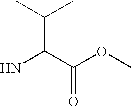 | 1.80 | 485.1 |
| 46 | I | Me | H | 0 | Et | 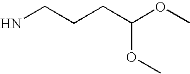 | 1.50 | 487.1 |
| 47 | I | Me | H | 0 | Et | 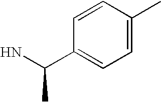 | 2.00 | 489.1 |
| 48 | I | Me | H | 0 | Et | 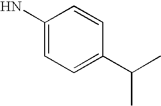 | 2.20 | 489.1 |
| 49 | I | Me | H | 0 | Et | 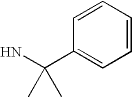 | 2.00 | 489.1 |
| 50 | I | Me | H | 0 | Et | 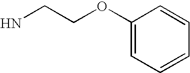 | 1.80 | 491.1 |
| 51 | I | Me | H | 0 | Et | 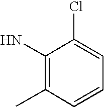 | 1.90 | 495.0 |
| 52 | I | Me | H | 0 | Et | 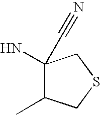 | 1.70 | 496.0 |
| 53 | I | Me | H | 0 | Et | 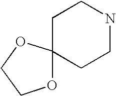 | 1.60 | 497.1 |
| 54 | I | Me | H | 0 | Et | 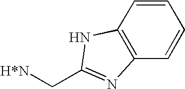 | 1.00 | 501.1 |
| 55 | I | Me | H | 0 | Et | 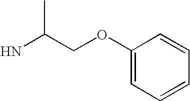 | 1.90 | 505.1 |
| 56 | I | Me | H | 0 | Et | 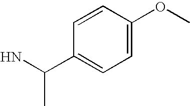 | 1.80 | 505.1 |
| 57 | I | Me | H | 0 | Et | 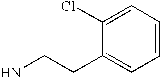 | 2.00 | 509.0 |
| 58 | I | Me | H | 0 | Et | 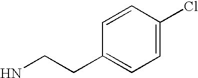 | 2.00 | 509.0 |
| 59 | I | Me | H | 0 | Et | 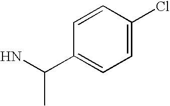 | 2.00 | 509.0 |
| 60 | I | Me | H | 0 | Et | 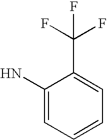 | 2.10 | 515.0 |
| 61 | I | Me | H | 0 | Et | 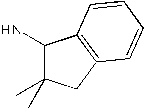 | 2.20 | 515.1 |
| 62 | I | Me | H | 0 | Et | 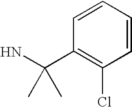 | 2.10 | 523.1 |
| 63 | I | Me | H | 0 | Et | 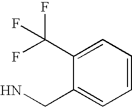 | 2.00 | 529.1 |
| 64 | I | Me | H | 0 | Et | 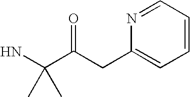 | 1.10 | 532.1 |
| 65 | I | Me | H | 0 | Et | 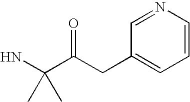 | 0.94 | 532.1 |
| 66 | I | Me | H | 0 | Et | 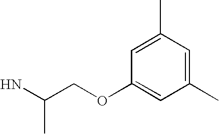 | 2.20 | 533.1 |
| 67 | I | Me | H | 0 | Et | 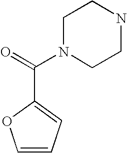 | 1.50 | 534.1 |
| 68 | I | Me | H | 0 | Et | 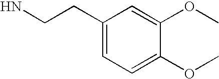 | 1.70 | 535.1 |
| 69 | I | Me | H | 0 | Et | 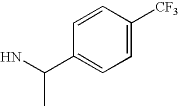 | 2.00 | 543.1 |
| 70 | I | Me | H | 0 | Et | 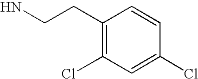 | 2.10 | 543.0 |
| 71 | I | Me | H | 0 | Et | 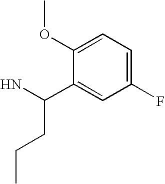 | 2.10 | 551.1 |
| 72 | I | Me | H | 0 | Et | 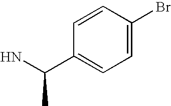 | 2.00 | 553.0 |
| 73 | I | Me | H | 0 | Et | 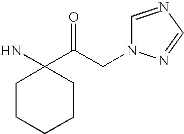 | 1.60 | 562.1 |
| 74 | I | Me | H | 0 | Et | 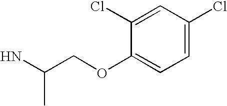 | 2.20 | 573.0 |
| 75 | I | Me | H | 0 | Et | 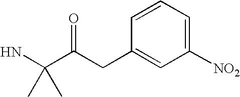 | 1.90 | 576.1 |
| 76 | I | Me | H | 0 | Et | 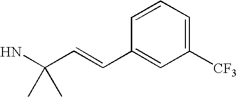 | 2.30 | 583.1 |
| 77 | I | Me | H | 0 | Et | 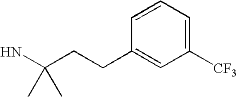 | 2.30 | 585.1 |
| 78 | I | Me | H | 0 | Et | 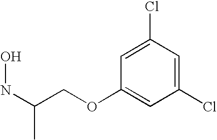 | 1.90 | 589.0 |
| 79 | I | Me | H | 0 | Et | 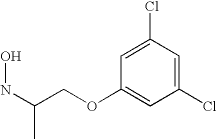 | 2.14 | 589.0 |
| 80 | I | Me | H | 0 | Et | 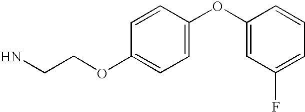 | 2.20 | 601.1 |
| 81 | I | Me | H | 0 | Et | 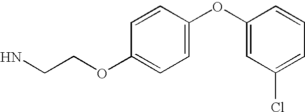 | 2.30 | 617.1 |
| 82 | Br | H | H | 1 | Me | 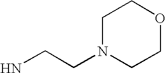 | 0.40 | 424.1 |
| 83 | Br | H | H | 1 | Me | 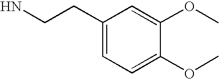 | 1.39 | 475.1 |
| 84 | Br | H | H | 1 | Me | 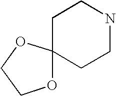 | 1.30 | 437.1 |
| 85 | Br | H | H | 1 | Me | 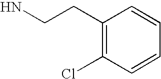 | 1.68 | 449.0 |
| 86 | Br | H | H | 1 | Me | 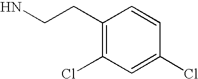 | 1.84 | 483.0 |
| 87 | Br | H | H | 1 | Me | 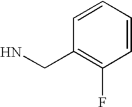 | 1.49 | 419.0 |
| 88 | Br | H | H | 1 | Me | 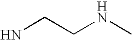 | 0.39 | 368.1 |
| 89 | Br | H | H | 1 | Me | 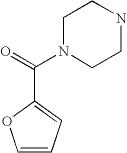 | 1.22 | 474.1 |
| 90 | Br | H | H | 1 | Me | 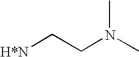 | 0.38 | 382.1 |
| 91 | Br | H | H | 1 | Me | 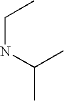 | 1.53 | 381.1 |
| 92 | Br | H | H | 1 | Me | 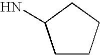 | 1.44 | 379.1 |
| 93 | Br | H | H | 1 | Me | 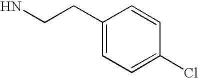 | 1.68 | 449.0 |
| 94 | Br | H | H | 1 | Me | 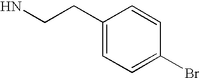 | 1.72 | 493.0 |
| 95 | Br | H | H | 1 | Me | 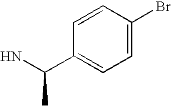 | 1.73 | 493.0 |
| 96 | Br | H | H | 1 | Me | 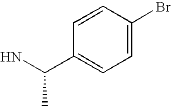 | 1.73 | 493.0 |
| 97 | Br | H | H | 1 | Me | 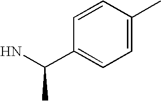 | 1.65 | 429.1 |
| 98 | Br | H | H | 1 | Me | 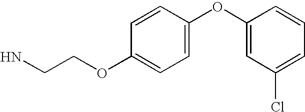 | 2.03 | 557.0 |
| 99 | Br | H | H | 1 | Me | 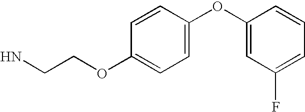 | 1.90 | 541.1 |
| 100 | Br | H | H | 1 | Me | 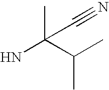 | 1.40 | 406.1 |
| 101 | Br | H | H | 1 | Me | 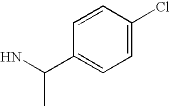 | 1.70 | 449.0 |
| 102 | Br | H | H | 1 | Me | 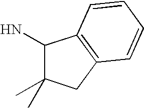 | 1.87 | 455.1 |
| 103 | Br | H | H | 1 | Me | 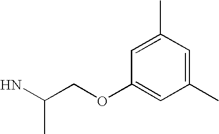 | 1.87 | 473.1 |
| 104 | Br | H | H | 1 | Me | 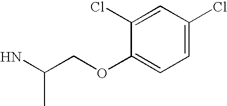 | 1.91 | 513.0 |
| 105 | Br | H | H | 1 | Me | 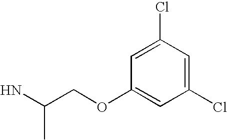 | 1.9 | 529.0 |
| 106 | Br | H | H | 1 | Me | 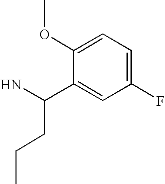 | 1.87 | 491.1 |
| 107 | Br | H | H | 1 | Me | 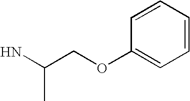 | 1.63 | 445.1 |
| 108 | Br | H | H | 1 | Me | 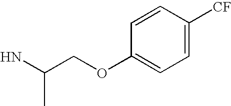 | 1.80 | 483.0 |
| 109 | Br | H | H | 1 | Me | 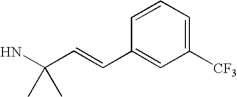 | 2.00 | 523.1 |
| 110 | Br | H | H | 1 | Me | 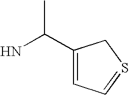 | 1.51 | 421.0 |
| 111 | Br | H | H | 1 | Me | 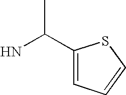 | 1.51 | 421.0 |
| 112 | Br | H | H | 1 | Me | 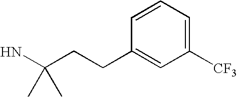 | 2.10 | 525.1 |
| 113 | Br | H | H | 1 | Me | 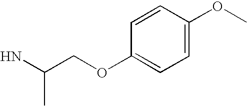 | 1.54 | 445.1 |
| 114 | Br | H | H | 1 | Me | 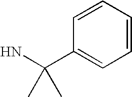 | 1.70 | 429.1 |
| 115 | Br | H | H | 1 | Me | 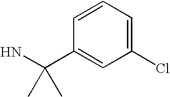 | 1.80 | 463.0 |
| 116 | Br | H | H | 1 | Me | 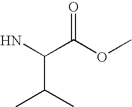 | 1.49 | 425.1 |
| 117 | Br | H | H | 1 | Me | 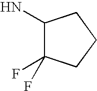 | 1.43 | 415.0 |
| 118 | Br | H | H | 1 | Me | 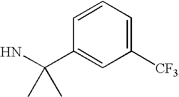 | 1.86 | 497.1 |
| 119 | Br | H | H | 1 | Me | 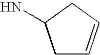 | 1.40 | 377.0 |
| 120 | Br | H | H | 1 | Me | 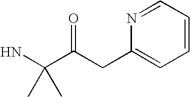 | 0.80 | 472.1 |
| 121 | Br | H | H | 1 | Me | 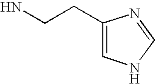 | 0.43 | 405.0 |
| 122 | Br | H | H | 1 | Me | 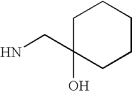 | 1.26 | 423.1 |
| 123 | Br | H | H | 1 | Me | 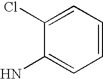 | 1.79 | 421.0 |
| 124 | Br | H | H | 1 | Me | 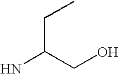 | 1.00 | 383.1 |
| 125 | Br | H | H | 1 | Me | 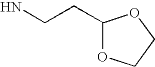 | 1.10 | 411.0 |
| 126 | Br | H | H | 1 | Me | 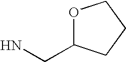 | 1.13 | 395.1 |
| 127 | Br | H | H | 1 | Me | 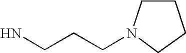 | 0.50 | 422.1 |
| 128 | Br | H | H | 1 | Me | 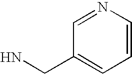 | 0.36 | 402.0 |
| 129 | Br | H | H | 1 | Me | 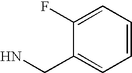 | 1.49 | 419.0 |
| 130 | Br | H | H | 1 | Me | 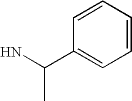 | 1.54 | 415.1 |
| 131 | Br | H | H | 1 | Me | 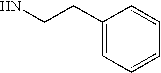 | 1.55 | 415.1 |
| 132 | Br | H | H | 1 | Me | 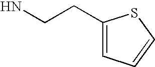 | 1.50 | 421.0 |
| 133 | Br | H | H | 1 | Me | 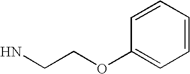 | 1.52 | 431.1 |
| 134 | Br | H | H | 1 | Me | 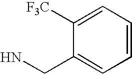 | 1.70 | 469.0 |
| 135 | Br | H | H | 1 | Me | 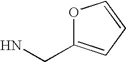 | 1.32 | 391.0 |
| 136 | Br | H | H | 1 | Me | 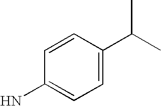 | 1.92 | 429.1 |
| 137 | Br | H | H | 1 | Me | 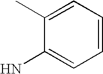 | 1.58 | 401.0 |
| 138 | Br | H | H | 1 | Me | 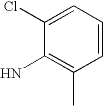 | 1.60 | 435.0 |
| 139 | I | H | H | 0 | Et | 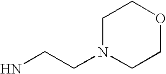 | 0.68 | 470.1 |
| 140 | I | H | H | 0 | Et | 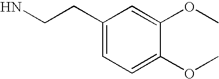 | 1.50 | 521.1 |
| 141 | I | H | H | 0 | Et | 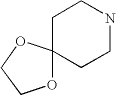 | 1.40 | 483.1 |
| 142 | I | H | H | 0 | Et | 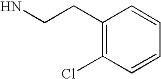 | 1.80 | 495.0 |
| 143 | I | H | H | 0 | Et | 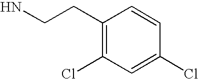 | 2.00 | 529.0 |
| 144 | I | H | H | 0 | Et | 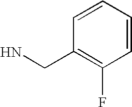 | 1.60 | 465.0 |
| 145 | I | H | H | 0 | Et | 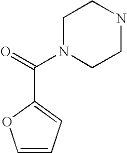 | 1.30 | 520.1 |
| 146 | I | H | H | 0 | Et | 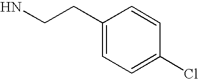 | 1.80 | 495.0 |
| 147 | I | H | H | 0 | Et | 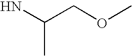 | 1.30 | 429.1 |
| 148 | I | H | H | 0 | Et | 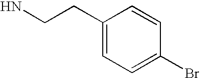 | 1.83 | 539.0 |
| 149 | I | H | H | 0 | Et | 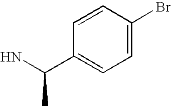 | 1.80 | 539.0 |
| 150 | I | H | H | 0 | Et | 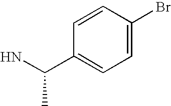 | 1.83 | 539.0 |
| 151 | I | H | H | 0 | Et | 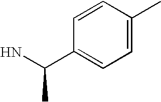 | 1.79 | 475.1 |
| 152 | I | H | H | 0 | Et | 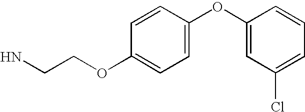 | 2.13 | 603.0 |
| 153 | I | H | H | 0 | Et | 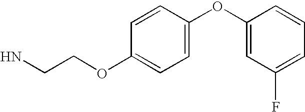 | 2.00 | 587.1 |
| 154 | I | H | H | 0 | Et | 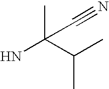 | 1.60 | 452.1 |
| 155 | I | H | H | 0 | Et | 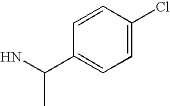 | 1.81 | 495.0 |
| 156 | I | H | H | 0 | Et | 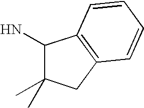 | 2.00 | 501.1 |
| 157 | I | H | H | 0 | Et | 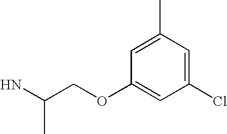 | 2.00 | 539.1 |
| 158 | I | H | H | 0 | Et | 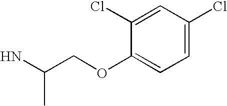 | 2.00 | 559.0 |
| 159 | I | H | H | 0 | Et | 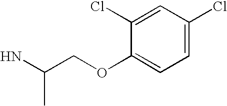 | 1.99 | 559.0 |
| 160 | I | H | H | 0 | Et | 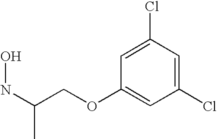 | 1.80 | 575.0 |
| 161 | I | H | H | 0 | Et | 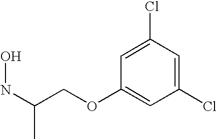 | 2.00 | 575.0 |
| 162 | I | H | H | 0 | Et | 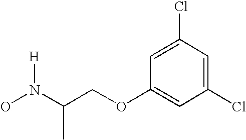 | 2.30 | 575.0 |
| 163 | I | H | H | 0 | Et | 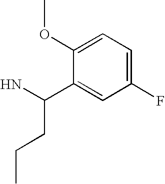 | 1.99 | 537.1 |
| 164 | I | H | H | 0 | Et | 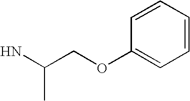 | 1.80 | 491.1 |
| 165 | I | H | H | 0 | Et | 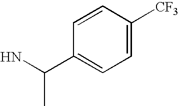 | 1.90 | 529.1 |
| 166 | I | H | H | 0 | Et | 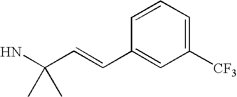 | 2.20 | 569.1 |
| 167 | I | H | H | 0 | Et | 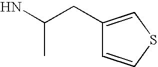 | 1.60 | 467.0 |
| 168 | I | H | H | 0 | Et | 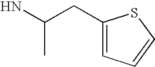 | 1.66 | 467.0 |
| 169 | I | H | H | 0 | Et | 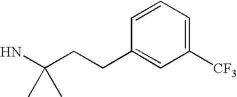 | 2.20 | 571.1 |
| 170 | I | H | H | 0 | Et | 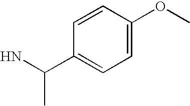 | 1.64 | 491.1 |
| 171 | I | H | H | 0 | Et | 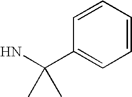 | 1.80 | 475.1 |
| 172 | I | H | H | 0 | Et | 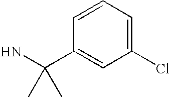 | 1.90 | 509.0 |
| 173 | I | H | H | 0 | Et | 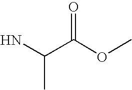 | 1.40 | 443.0 |
| 174 | I | H | H | 0 | Et | 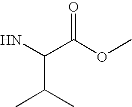 | 1.60 | 471.1 |
| 175 | I | H | H | 0 | Et | 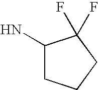 | 1.60 | 461.0 |
| 176 | I | H | H | 0 | Et | 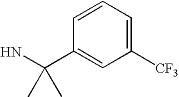 | 1.99 | 543.1 |
| 177 | I | H | H | 0 | Et | 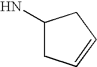 | 1.50 | 423.0 |
| 178 | I | H | H | 0 | Et | 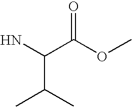 | 1.60 | 471.1 |
| 179 | I | H | H | 0 | Et | 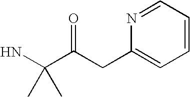 | 0.98 | 518.1 |
| 180 | I | H | H | 0 | Et | 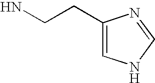 | 0.70 | 451.1 |
| 181 | I | H | H | 0 | Et | 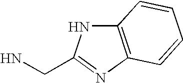 | 0.87 | 487.1 |
| 182 | I | H | H | 0 | Et | 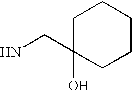 | 1.40 | 469.1 |
| 183 | I | H | H | 0 | Et | 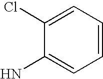 | 2.00 | 467.0 |
| 184 | I | H | H | 0 | Et | 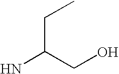 | 1.17 | 429.1 |
| 185 | I | H | H | 0 | Et | 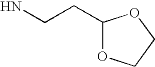 | 1.27 | 457.1 |
| 186 | I | H | H | 0 | Et | 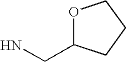 | 1.30 | 441.1 |
| 187 | I | H | H | 0 | Et | 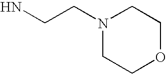 | 0.73 | 470.1 |
| 188 | I | H | H | 0 | Et | 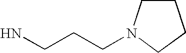 | 0.73 | 468.1 |
| 189 | I | H | H | 0 | Et | 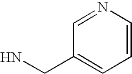 | 0.77 | 448.0 |
| 190 | I | H | H | 0 | Et | 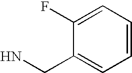 | 1.64 | 465.0 |
| 191 | I | H | H | 0 | Et | 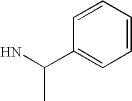 | 1.68 | 461.1 |
| 192 | I | H | H | 0 | Et | 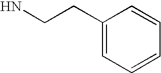 | 1.69 | 461.1 |
| 193 | I | H | H | 0 | Et | 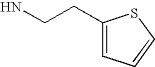 | 1.60 | 467.0 |
| 194 | I | H | H | 0 | Et | 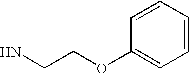 | 1.60 | 477.1 |
| 195 | I | H | H | 0 | Et | 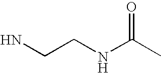 | 0.95 | 442.1 |
| 196 | I | H | H | 0 | Et | 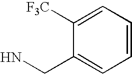 | 1.80 | 515.0 |
| 197 | I | H | H | 0 | Et | 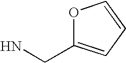 | 1.50 | 437.0 |
| 198 | I | H | H | 0 | Et | 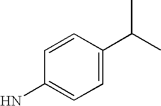 | 2.05 | 475.1 |
| 199 | I | H | H | 0 | Et | 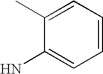 | 1.73 | 447.0 |
| 200 | I | H | H | 0 | Et | 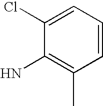 | 1.70 | 481.0 |
| 201 | Br | H | H | 0 | Et | 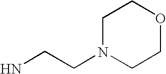 | 0.50 | 422.1 |
| 202 | Br | H | H | 0 | Et | 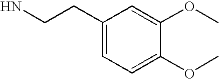 | 1.60 | 473.1 |
| 203 | Br | H | H | 0 | Et | 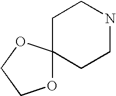 | 1.40 | 435.1 |
| 204 | Br | H | H | 0 | Et | 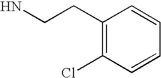 | 1.80 | 447.0 |
| 205 | Br | H | H | 0 | Et | 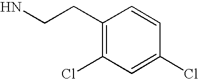 | 2.00 | 481.0 |
| 206 | Br | H | H | 0 | Et | 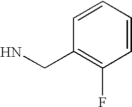 | 1.70 | 417.1 |
| 207 | Br | H | H | 0 | Et | 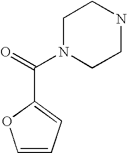 | 1.40 | 472.1 |
| 208 | Br | H | H | 0 | Et | 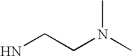 | 0.60 | 380.1 |
| 209 | Br | H | H | 0 | Et | 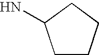 | 1.60 | 377.1 |
| 210 | Br | H | H | 0 | Et | 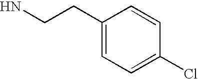 | 1.80 | 447.0 |
| 211 | Br | H | H | 0 | Et | 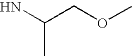 | 1.40 | 381.1 |
| 212 | Br | H | H | 0 | Et | 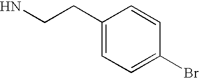 | 1.90 | 491.0 |
| 213 | Br | H | H | 0 | Et | 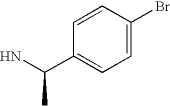 | 1.90 | 491.0 |
| 214 | Br | H | H | 0 | Et | 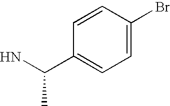 | 1.90 | 491.0 |
| 215 | Br | H | H | 0 | Et | 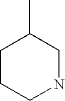 | 1.70 | 391.1 |
| 216 | Br | H | H | 0 | Et | 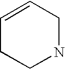 | 1.50 | 375.1 |
| 217 | Br | H | H | 0 | Et | 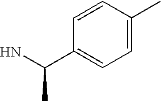 | 1.80 | 427.1 |
| 218 | Br | H | H | 0 | Et | 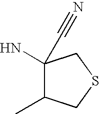 | 1.60 | 434.0 |
| 219 | Br | H | H | 0 | Et | 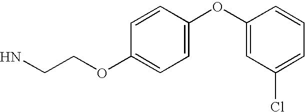 | 2.20 | 555.1 |
| 220 | Br | H | H | 0 | Et | 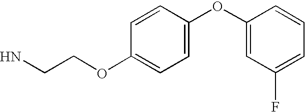 | 2.10 | 539.1 |
| 221 | Br | H | H | 0 | Et | 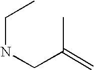 | 1.80 | 391.1 |
| 222 | Br | H | H | 0 | Et | 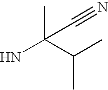 | 1.60 | 404.1 |
| 223 | Br | H | H | 0 | Et | 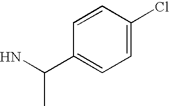 | 1.80 | 447.0 |
| 224 | Br | H | H | 0 | Et | 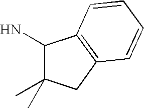 | 2.00 | 453.1 |
| 225 | Br | H | H | 0 | Et | 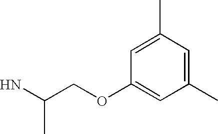 | 2.00 | 471.1 |
| 226 | Br | H | H | 0 | Et | 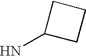 | 1.50 | 363.1 |
| 227 | Br | H | H | 0 | Et | 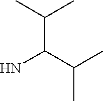 | 1.90 | 407.1 |
| 228 | Br | H | H | 0 | Et | 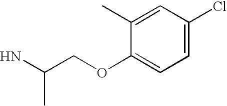 | 2.00 | 491.1 |
| 229 | Br | H | H | 0 | Et | 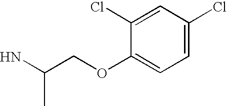 | 2.00 | 511.0 |
| 230 | Br | H | H | 0 | Et | 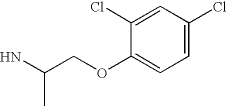 | 2.10 | 511.0 |
| 231 | Br | H | H | 0 | Et | 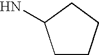 | 1.60 | 377.1 |
| 232 | Br | H | H | 0 | Et | 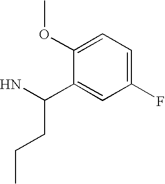 | 2.00 | 489.1 |
| 233 | Br | H | H | 0 | Et | 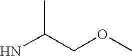 | 1.40 | 381.1 |
| 234 | Br | H | H | 0 | Et | 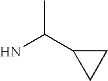 | 1.60 | 377.1 |
| 235 | Br | H | H | 0 | Et | 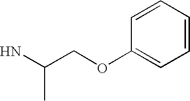 | 1.80 | 443.1 |
| 236 | Br | H | H | 0 | Et | 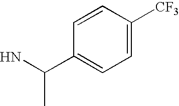 | 1.90 | 481.1 |
| 237 | Br | H | H | 0 | Et | 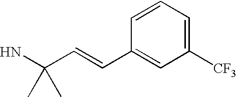 | 2.20 | 521.1 |
| 238 | Br | H | H | 0 | Et | 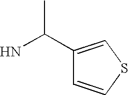 | 1.70 | 419.0 |
| 239 | Br | H | H | 0 | Et | 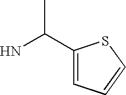 | 1.70 | 419.0 |
| 240 | Br | H | H | 0 | Et | 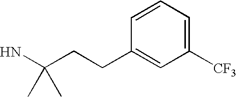 | 2.20 | 523.1 |
| 241 | Br | H | H | 0 | Et | 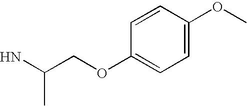 | 1.70 | 443.1 |
| 242 | Br | H | H | 0 | Et | 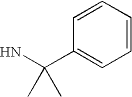 | 1.90 | 427.1 |
| 243 | Br | H | H | 0 | Et | 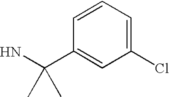 | 2.00 | 461.1 |
| 244 | Br | H | H | 0 | Et | 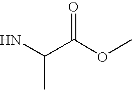 | 1.40 | 395.1 |
| 245 | Br | H | H | 0 | Et | 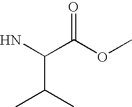 | 1.70 | 423.1 |
| 246 | Br | H | H | 0 | Et | 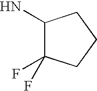 | 1.60 | 413.1 |
| 247 | Br | H | H | 0 | Et | 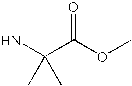 | 1.50 | 409.1 |
| 248 | Br | H | H | 0 | Et | 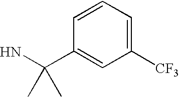 | 2.00 | 495.1 |
| 249 | Br | H | H | 0 | Et | 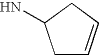 | 1.60 | 375.1 |
| 250 | Br | H | H | 0 | Et | 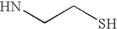 | 2.00 | 369.0 |
| 251 | Br | H | H | 0 | Et | 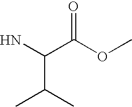 | 1.70 | 423.1 |
| 252 | Br | H | H | 0 | Et | 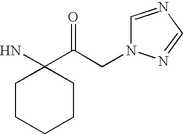 | 1.50 | 500.1 |
| 253 | Br | H | H | 0 | Et | 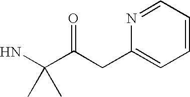 | 1.00 | 470.1 |
| 254 | Br | H | H | 0 | Et | 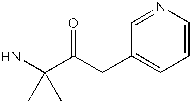 | 0.90 | 470.1 |
| 255 | Br | H | H | 0 | Et | 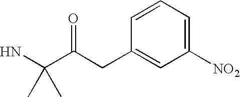 | 1.80 | 514.1 |
| 256 | Br | H | H | 0 | Et | 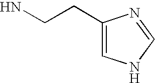 | 0.50 | 403.1 |
| 257 | Br | H | H | 0 | Et | 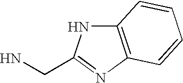 | 0.80 | 439.1 |
| 258 | Br | H | H | 0 | Et | 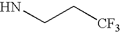 | 1.60 | 405.0 |
| 259 | Br | H | H | 0 | Et | 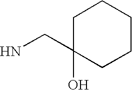 | 1.50 | 421.1 |
| 260 | Br | H | H | 0 | Et | 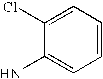 | 2.00 | 419.0 |
| 261 | Br | H | H | 0 | Et | 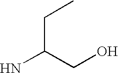 | 1.20 | 381.1 |
| 262 | Br | H | H | 0 | Et | 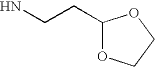 | 1.30 | 409.1 |
| 263 | Br | H | H | 0 | Et | 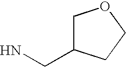 | 1.40 | 393.1 |
| 264 | Br | H | H | 0 | Et | 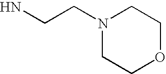 | 0.60 | 422.1 |
| 265 | Br | H | H | 0 | Et | 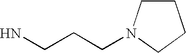 | 0.60 | 420.1 |
| 266 | Br | H | H | 0 | Et | 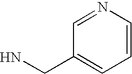 | 0.70 | 400.1 |
| 267 | Br | H | H | 0 | Et | 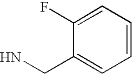 | 1.70 | 417.1 |
| 268 | Br | H | H | 0 | Et | 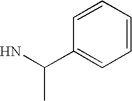 | 1.70 | 413.1 |
| 269 | Br | H | H | 0 | Et | 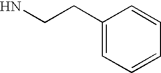 | 1.70 | 413.1 |
| 270 | Br | H | H | 0 | Et | 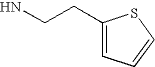 | 1.70 | 419.0 |
| 271 | Br | H | H | 0 | Et | 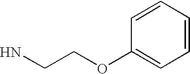 | 1.70 | 429.1 |
| 272 | Br | H | H | 0 | Et | 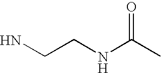 | 1.00 | 394.1 |
| 273 | Br | H | H | 0 | Et | 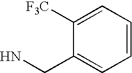 | 1.90 | 467.1 |
| 274 | Br | H | H | 0 | Et | 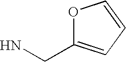 | 1.50 | 389.0 |
| 275 | Br | H | H | 0 | Et | 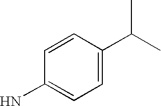 | 2.10 | 427.1 |
| 276 | Br | H | H | 0 | Et | 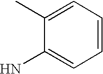 | 1.80 | 399.1 |
| 277 | Br | H | H | 0 | Et | 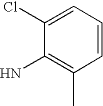 | 1.80 | 433.0 |
| 278 | Br | H | H | 0 | Et | 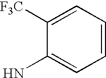 | 2.00 | 453.0 |

### EXAMPLE 10

This Example illustrates the fungicidal properties of compounds of formula (I).

The compounds were tested in a leaf disk assay, with methods described below. The test compounds were dissolved in DMSO and diluted into water to 200 ppm. In the case of the test on Pythium ultimum, they were dissolved in DMSO and diluted into water to 20 ppm.

Erysiphe graminis f.sp. tritici (wheat powdery mildew): Wheat leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Puccinia recondita f.sp. tritici (wheat brown rust): Wheat leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed nine days after inoculation as preventive fungicidal activity.

Septoria nodorum (wheat glume blotch): Wheat leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Pyrenophora teres (barley net blotch): Barley leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Pyricularia oryzae (rice blast): Rice leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Botrytis cinerea (grey mould): Bean leaf disks were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Phytophthora infestans (late blight of potato on tomato): Tomato leaf disks were placed on water agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Plasmopara viticola (downy mildew of grapevine): Grapevine leaf disks were placed on agar in a 24-well plate and sprayed a solution of the test compound. After allowing to dry completely, for between 12 and 24 hours, the leaf disks were inoculated with a spore suspension of the fungus. After appropriate incubation the activity of a compound was assessed seven days after inoculation as preventive fungicidal activity.

Septoria tritici (leaf blotch): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24 C and the inhibition of growth was determined photometrically after 72 hrs.

Fusarium culmorum (root rot): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24 C and the inhibition of growth was determined photometrically after 48 hrs.

Pythium ultimum (Damping off): Mycelial fragments of the fungus, prepared from a fresh liquid culture, were mixed into potato dextrose broth. A solution of the test compound in dimethyl sulphoxide was diluted with water to 20 ppm then placed into a 96-well microtiter plate and the nutrient broth containing the fungal spores was added. The test plate was incubated at 24° C. and the inhibition of growth was determined photometrically after 48 hours.

The following compounds (number of compound first, followed by table number in brackets) gave at least 60% control of the following fungal infection at 200 ppm:

Plasmopara viticola, compounds, 12 (95), 12 (99), 12 (156), 12 (159), 38 (154), 47 (91), 47 (99), 47 (118), 47 (156), 52 (90), 52 (118), 52 (156), 60 (154), 85 (118), 85 (158), 34 (161), 25 (161), 23 (161), 16 (161), 11 (161), 8 (161), 7 (161), 6 (161), 5 (161), 2 (161), 1 (161), 78 (161), 77 (161), 74 (161), 72 (161), 71 (161), 70 (161), 67 (161), 65 (161), 47 (161), 44 (161), 42 (161), 32 (162), 64 (162), 90 (162), 91 (162), 115 (162), 177 (162), 222 (162), 247 (162).

Phytophthora infestans, compounds, 12 (156), 12 (158), 12 (159), 47 (154), 47 (156), 52 (118), 34 (161), 25 (161), 6 (161), 2 (161), 78 (161), 77 (161), 76 (161), 75 (161), 67 (161), 50 (161), 48 (161), 44 (161), 32 (162), 122 (162), 179 (162), 189 (162).

Botrytis cinerea, compounds, 24 (161), 8 (161), 6 (161), 5 (161), 78 (161), 73 (161), 72 (161), 60 (161), 57 (161), 56 (161), 54 (161), 53 (161), 51 (161), 49 (161), 48 (161), 47 (161), 46 (161), 41 (161), 81 (161), 4 (162), 150 (162), 155 (162), 169 (162), 174 (162), 176 (162), 177 (162), 185 (162), 246 (162).

Pyrenophora teres, compounds, 77 (161), 76 (161), 67 (161), 51 (161), 47 (161).

Erysiphe graminis f.sp. tritici, 12 (91), 12 (94), 12 (95), 12 (99), 12 (118), 12 (119), 12 (154), 12 (155), 12 (156), 12 (159), 39 (90), 39 (94), 47 (90), 47 (91), 47 (94), 47 (99), 47 (118), 47 (155), 47 (156), 52 (91), 52 (94), 52 (99), 52 (118), 52 (154), 52 (155), 52 (158), 85 (118), 264 (158), 275 (158), 290 (154), 35 (161), 34 (161), 33 (161), 31 (161), 30 (161), 29 (161), 27 (161), 26 (161), 25 (161), 24 (161), 23 (161), 22 (161), 11 (161), 10 (161), 8 (161), 7 (161), 6 (161), 5 (161), 4 (161), 3 (161), 2 (161), 1 (161), 77 (161), 76 (161), 74 (161), 73 (161), 72 (161), 71 (161), 70 (161), 69 (161), 68 (161), 67 (161), 65 (161), 64 (161), 62 (161), 61 (161), 60 (161), 59 (161), 58 (161), 57 (161), 56 (161), 55 (161), 54 (161), 53 (161), 52 (161), 51 (161), 50 (161), 49 (161), 48 (161), 47 (161), 46 (161), 45 (161), 44 (161), 43 (161), 79 (161), 80 (161), 2 (162), 3 (162), 16 (162), 25 (162), 32 (162), 33 (162), 46 (162), 64 (162), 77 (162), 91 (162), 147 (162), 154 (162), 170 (162), 177 (162), 183 (162), 185 (162), 189 (162), 226 (162).

Pyricularia oryzae, compounds 12 (159), 287 (155); 26 (161), 25 (161), 24 (161), 23 (161), 8 (161), 6 (161), 77 (161), 76 (161), 67 (161), 51 (161), 47 (161), 109 (162), 110 (162), 117 (162), 138 (162), 177 (162).

Puccinia recondita f.sp. tritici, compounds 47 (94), 47 (118), 52 (118), 85 (158), 23 (161), 11 (161), 6 (161), 74 (161), 72 (161), 71 (161), 67 (161), 58 (161), 54 (161), 16 (162).

Septoria nodorum, compounds, 12 (119), 12 (159), 47 (91), 47 (94), 47 (99), 47 (118), 47 (156), 85 (118), 26 (161), 25 (161), 23 (161), 11 (161), 77 (161), 74 (161), 73 (161), 72 (161), 71 (161), 67 (161), 54 (161), 48 (161), 47 (161), 46 (161), 16 (162), 32 (162), 107 (162), 183 (162), 190 (162), 265 (162).

Septoria tritici, compounds, 12 (90), 12 (91), 12 (94), 12 (99), 12 (103), 12 (118), 12 (119), 12 (155), 12 (156), 12 (159), 38 (158), 39 (94), 39 (118), 47 (91), 47 (99), 47 (118), 47 (155), 52 (91), 52 (94), 52 (99), 52 (118), 85 (118), 85 (158), 95 (155), 181 (154), 181 (155), 189 (155), 189 (158), 190 (155), 275 (158), 290 (154), 35 (161), 33 (161), 32 (161), 31 (161), 29 (161), 27 (161), 26 (161), 25 (161), 24 (161), 23 (161), 22 (161), 11 (161), 10 (161), 9 (161), 8 (161), 6 (161), 5 (161), 3 (161), 2 (161), 1 (161), 78 (161), 77 (161), 76 (161), 74 (161), 73 (161), 72 (161), 71 (161), 70 (161), 68 (161), 67 (161), 62 (161), 60 (161), 59 (161), 58 (161), 57 (161), 56 (161), 55 (161), 54 (161), 53 (161), 52 (161), 51 (161), 50 (161), 49 (161), 48 (161), 47 (161), 46 (161), 81 (161), 1 (162), 2 (162), 3 (162), 4 (162), 5 (162), 6 (162), 7 (162), 8 (162), 9 (162), 10 (162), 11 (162), 13 (162), 16 (162), 17 (162), 18 (162), 21 (162), 23 (162), 25 (162), 26 (162), 32 (162), 33 (162), 34 (162), 36 (162), 46 (162), 49 (162), 52 (162), 54 (162), 56 (162), 59 (162), 64 (162), 65 (162), 67 (162), 87 (162), 92 (162), 101 (162), 114 (162), 115 (162), 130 (162), 147 (162), 154 (162), 155 (162), 167 (162), 171 (162), 177 (162), 183 (162), 191 (162), 197 (162), 199 (162), 226 (162), 234 (162), 268 (162).

Fusarium culmorum, compounds, 12 (91), 12 (95), 12 (103), 12 (118), 12 (119), 12 (155), 12 (159), 39 (118), 52 (158), 275 (158), 27 (161), 26 (161), 25 (161), 22 (161), 8 (161), 1 (161), 73 (161), 53 (161), 48 (161), 46 (161), 43 (161), 3 (162), 32 (162).

The following compounds (number of compound first, followed by table number in brackets) gave at least 60% control of the following fungal infection at 20 ppm:

Pythium ultimum, compounds 12 (118), 12 (158), 12 (159), 39 (118), 39 (158), 47 (118), 52 (118), 34 (161), 30 (161), 25 (161), 1 (161), 78 (161), 77 (161), 76 (161), 75 (161), 70 (161), 50 (161), 49 (161), 48 (161), 43 (161), 37 (161), 36 (161), 23 (162), 32 (162), 54 (162), 64 (162), 65 (162), 147 (162), 177 (162), 179 (162), 181 (162), 185 (162), 189 (162), 195 (162), 253 (162), 254 (162).

1. A compound of the general formula I 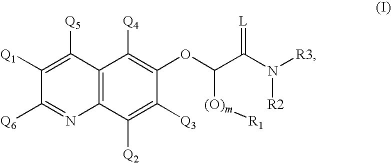
wherein
Q¹, Q², Q³, Q⁴, Q⁵ and Q⁶ independently of each other, are hydrogen halogen, cyano, nitro, azido, optionally substituted C₁₋₆ alkyl, optionally substituted C₃₋₆ cycloalkyl, optionally substituted C₃₋₆ cycloalkyl(C₁₋₄)alkyl, optionally substituted C₂₋₆ alkenyl, optionally substituted C₂₋₆ alkynyl, optionally substituted C₁₋₆ alkoxy, optionally substituted C₂₋₆ alkenyloxy, optionally substituted C₂₋₆ alkynyloxy, optionally substituted aryl, optionally substituted aryloxy, optionally substituted aryl(C₁₋₆)alkyl, optionally substituted aryl(C₁₋₆)alkoxy, optionally substituted heteroaryl, optionally substituted heteroaryloxy, optionally substituted heteroaryl(C₁₋₆)alkyl, optionally substituted heteroaryl(C₁₋₆)alkoxy, —SF₅ or —S(O)ᵤ(C₁₋₆)alkyl, wherein u is 0, 1 or 2 and the alkyl group is optionally substituted with halogen, or
Q¹, Q², Q³, Q⁴, Q⁵ and Q⁶, independently of each other, are —OSO₂(C₁₋₄)alkyl, wherein the alkyl group is optionally substituted with halogen, or
Q¹, Q², Q³, Q⁴, Q⁵ and Q⁶, independently of each other, are —CONR^{u}R^{v}, —COR^{u}, —CO₂R^{u}, —CR^{u}═NR^{v}, —NR^{u}COR^{v}, —NR^{u}CO₂R^{v}, —SO₂NR^{u}R^{v} or —NR^{u}SO₂R^{w}, wherein R^{w} is optionally substituted C₁₋₆ alkyl and R^{u} and R^{v} independently of each other, are hydrogen or C₁₋₆ alkyl optionally substituted with halogen, or, in the case of —CONR^{u}R^{v} or —SO₂NR^{u}R^{v}, R^{u}R^{v} may join to form a 5- or 6-membered carbocyclic or heterocyclic ring containing a heteroatom selected from sulfur, oxygen and NR^{o}, wherein R^{o} is hydrogen or optionally substituted C₁₋₆alkyl, or, in the case of —CR^{u}═NR^{v}, R^{v} is hydrogen, hydroxyl, or C₁₋₆alkoxy,
R¹ is C₁₋₄ alkyl, C₃₋₅ cycloalkyl, C₂₋₄ alkenyl or C₂₋₄ alkynyl in which the alkyl, alkenyl and alkynyl groups are optionally substituted on their terminal carbon atom with one, two or three halogen atoms, with a cyano group, with a C₁₋₄ alkylcarbonyl group, with a C₁₋₄ alkoxycarbonyl group or with a hydroxy group, or R₁ is alkoxyalkyl, alkylthioalkyl, alkylsulphinylalkyl or alkylsulphonylalkyl in which the total number of carbon atoms is 2 or 3, or R₁ is a straight-chain C₁₋₄ alkoxy group;
R² is hydrogen, C₁₋₈ alkyl, C₃₋₄ cycloalkyl, C₂₋₈ alkenyl, cyano(C₁₋₄)alkyl, C₁₋₄ alkoxy(C₁₋₄)-alkyl, C₁₋₄ alkoxy(C₁₋₄)alkoxy(C₁₋₄)alkyl or benzyloxy(C₁₋₄)alkyl, wherein the phenyl ring is optionally substituted with C₁₋₄ alkoxy,
R³ is —(CR^{a}R^{b})ₚ(CR^{c}R^{d})_{q}(X)ᵣ(CR^{e}R^{f})ₛR⁴, wherein
R^{a}, R^{b}, R^{c}, R^{d}, R^{e} and R^{f}, independently of each other, are hydrogen, C₁₋₄ alkyl, halogen, cyano, hydroxy, C₁₋₄ alkoxy or C₁₋₄ alkoxycarbonyl, or
R^{a}R^{b}, R^{c}R^{d} or R^{e}R^{f} may join to form a 3 to 8 membered carbocyclic or heterocyclic ring containing a heteroatom selected from sulfur, oxygen and NR^{o}, wherein R^{o} is hydrogen or optionally substituted C₁₋₆alkyl,
X is (CO), (CO)O, O(CO), O, S(O)ₜ, wherein t is 0, 1 or 2, or X is NH or N(C₁₋₆)alkyl,
p, r and s, independently of each other, are 0 or 1,
q is 0, 1 or 2,
R⁴ is optionally substituted C₁₋₆ alkyl, optionally substituted C₂₋₆ alkenyl or when at least one of p, q, r and s is 1, R⁴ is —CH₂—C≡C—R⁵, wherein
R⁵ is hydrogen, C₁₋₈ alkyl optionally substituted with halogen, hydroxy, C₁₋₆ alkoxy, C₁₋₃ alkoxy(C₁₋₃)alkoxy, cyano, C₁₋₄ alkylcarbonyloxy, aminocarbonyloxy, mono- or di(C₁₋₄)-alkylaminocarbonyloxy, tri(C₁₋₄)alkylsilyloxy or —S(O)_{g}(C₁₋₆)alkyl, wherein g is 0, 1 or 2, or
R⁵ is C₃₋₆ cycloalkyl optionally substituted with halogen, hydroxy, C₁₋₆ alkoxy, C₁₋₃ alkoxy-(C₁₋₃)alkoxy, cyano, C₁₋₄ alkylcarbonyloxy, aminocarbonyloxy, mono- or di(C₁₋₄)alkyl-aminocarbonyloxy, tri(C₁₋₄)alkylsilyloxy or —S(O)_{g}(C₁₋₆)alkyl, wherein g is 0, 1 or 2, or
R⁵ is C₃₋₆ cycloalkyl(C₁₋₄)alkyl, wherein the alkyl and/or cycloalkyl moiety is optionally substituted with halogen, hydroxy, C₁₋₆ alkoxy, C₁₋₃ alkoxy(C₁₋₃)alkoxy, cyano, C₁₋₄ alkylcarbonyloxy, aminocarbonyloxy, mono- or di(C₁₋₄)alkylaminocarbonyloxy, tri(C₁₋₄)alkylsilyloxy or —S(O)_{g}(C₁₋₆)alkyl, wherein g is 0, 1 or 2, or
R⁵ is optionally substituted aryl, optionally substituted aryl(C₁₋₄)alkyl, optionally substituted aryloxy(C₁₋₄)alkyl, optionally substituted heteroaryl or optionally substituted heteroaryl(C₁₋₄)alkyl or optionally substituted heteroaryloxy(C₁₋₄)alkyl, or
R⁴ is optionally substituted C₃₋₆ cycloalkyl, optionally substituted C₅₋₆ cycloalkenyl, optionally substituted aryl, optionally substituted heteroaryl or an optionally substituted 5- to 8-membered ring optionally containing a heteroatom selected from sulfur, oxygen or NR⁰, wherein R^{o} is hydrogen or optionally substituted C₁₋₆alkyl, or
R² and R³ may join to form a 5- or 6-membered ring optionally substituted with halogen, C₁₋₄ alkyl, mono- or di-(C₁₋₄)alkylaminocarbonyl, and optionally containing a heteroatom selected from sulphur, oxygen and NR⁰⁰, wherein R⁰⁰ is C₁₋₄ alkyl optionally substituted with halogen, C₁₋₆ alkoxy or cyano, or R⁰⁰ is phenyl optionally substituted with nitro, C₁₋₄ alkyl, halo(C₁₋₄)alkyl, C₁₋₄ alkylcarbonyl or heteroaryl, or R² and R³ may join to form an optionally substituted 6,6-membered bicycle,
L is sulfur or oxygen, and
m is 0 or 1; and
salts and N-oxides of the compounds of the formula I.

2. Compounds according to claim 1, wherein Q² is hydrogen, C₁₋₄ alkyl or halogen, Q¹, Q³, Q⁴, Q⁵ and Q⁶ are as defined in claim 1.

3. Compounds according to claim 2, wherein Q² is methyl or ethyl.

4. Compounds according to claim 1, wherein Q¹ is halogen, aryl or heteroaryl, Q² is hydrogen, C₁₋₄ alkyl or halogen and Q³, Q⁴, Q⁵ and Q⁶ are as defined in claim 1.

5. Compounds according to claim 4, wherein Q² is methyl or ethyl.

6. Compounds according to claim 1, wherein Q¹ is aryl, Q² is hydrogen, C₁₋₄ alkyl or halogen and Q³, Q⁴, Q⁵ and Q⁶ is as defined in claim 1.

7. Compounds according to claim 6, wherein Q² is methyl or ethyl.

8. Compounds according to claim 1, wherein Q¹ is heteroaryl, Q² is hydrogen, C₁₋₄ alkyl or halogen and Q³, Q⁴, Q⁵ and Q⁶ is as defined in claim 1.

9. Compounds according to claim 8, wherein Q² is methyl or ethyl.

10. Compounds according to claim 1, wherein Q¹ and Q³, independently of each other, are hydrogen or halogen and Q², Q⁴, Q⁵ and Q⁶ are hydrogen.

11. Compounds according to claim 10, wherein Q¹ and Q³, independently of each other, are fluoro, chloro, bromo or iodo.

12. Compounds according to claim 10, wherein Q¹ is chloro, bromo or iodo, and Q³ is fluoro or chloro.

13. Compounds according to claim 1, wherein Q¹ is aryl or heteroaryl, Q², Q⁴, Q⁵ and Q⁶ are hydrogen and Q³ is hydrogen or halogen.

14. Compounds according to claim 13, wherein Q¹ is thiophen-2-yl, thiophen-3-yl, halo, or halo or alkoxy substituted phenyl, or halo or alkoxy substituted pyridyl.

15. Compounds according to claim 13, wherein Q³ is hydrogen, fluoro or chloro.

16. Compounds according to claim 1, wherein Q¹, Q², Q⁴, Q⁵ and Q⁶ are hydrogen and Q³ is hydrogen, halogen or optionally substituted alkyl.

17. Compounds according to claim 16, wherein Q³ is hydrogen, fluoro or chloro.

18. Compounds according to claim 1, wherein Q¹ is halogen, Q², Q⁴, Q⁵ and Q⁶ are hydrogen and Q³ is hydrogen or optionally substituted alkyl.

19. Compounds according to claim 18, wherein Q¹ is chloro, bromo or iodo.

20. Compounds according to claim 18, wherein Q³ is methyl.

21. Compounds according to claim 1, wherein Q¹ and Q² are halogen and Q³ is hydrogen or optionally substituted alkyl and Q⁴, Q⁵ and Q⁶ are hydrogen.

22. Compounds according to claim 21, wherein Q¹ is chloro, bromo or iodo.

23. Compounds according to claim 21, wherein Q³ is methyl.

24. Compounds according to claim 1, wherein Q¹ is bromo and Q², Q³, Q⁴, Q⁵ and Q⁶, independently of each other, are hydrogen, C₁₋₄ alkyl or halogen.

25. Compounds according to claim 24, wherein Q² is halogen and Q³, Q⁴, Q⁵ and Q⁶ are hydrogen.

26. Compounds according to claim 24, wherein Q² is methyl or ethyl and Q³, Q⁴, Q⁵ and Q⁶ are hydrogen.

27. Compounds according to claim 24, wherein Q³ is fluoro or chloro and Q², Q⁴, Q⁵ and Q⁶ are hydrogen.

28. Compounds according to claim 1, wherein Q¹ is iodo and Q², Q³, Q⁴, Q⁵ and Q⁶, independently of each other, are hydrogen, C₁₋₄ alkyl or halogen.

29. Compounds according to claim 28, wherein Q² is halogen and Q³, Q⁴, Q⁵ and Q⁶ are hydrogen.

30. Compounds according to claim 28, wherein Q² is methyl or ethyl and Q³, Q⁴, Q⁵ and Q⁶ are hydrogen.

31. Compounds according to claim 28, wherein Q³ is fluoro or chloro and Q², Q⁴, Q⁵ and Q⁶ are hydrogen.

32. Compounds according to claim 1 wherein Q¹ is chloro and Q², Q³, Q⁴, Q⁵ and Q⁶, independently of each other, are hydrogen, C₁₋₄ alkyl or halogen.

33. Compounds according to claim 32, wherein Q² is halogen and Q³, Q⁴, Q⁵ and Q⁶ are hydrogen.

34. Compounds according to claim 32, wherein Q² is methyl or ethyl and Q³, Q⁴, Q⁵ and Q⁶ are hydrogen.

35. Compounds according to claim 32, wherein Q³ is fluoro or chloro and Q², Q⁴, Q⁵ and Q⁶ are hydrogen.

36. Compounds according to claim 1, wherein Q¹ is chloro, bromo or iodo.

37. Compounds according to claim 1, wherein Q¹ is fluoro.

38. Compounds according to claim 1, wherein Q³ is hydrogen or halogen.

39. Compounds according to claim 38, wherein Q³ is hydrogen, fluoro or chloro.

40. Compounds according to claim 39, wherein Q³ is fluoro.

41. Compounds according to claim 1, wherein Q¹ is bromo, Q², Q⁴, Q⁵ and Q⁶ are hydrogen and Q³ is hydrogen, fluoro or chloro.

42. Compounds according to claim 41, wherein Q³ is fluoro.

43. Compounds according to claim 41, wherein Q³ is chloro.

44. Compounds according to claim 41, wherein Q³ is hydrogen.

45. Compounds according to claim 1, wherein Q¹ is iodo, Q², Q⁴, Q⁵ and Q⁶ are hydrogen and Q³ is hydrogen, fluoro or chloro.

46. Compounds according to claim 45, wherein Q¹ is iodo, Q², Q⁴, Q⁵ and Q⁶ are hydrogen and Q³ is fluoro.

47. Compounds according to claim 45, wherein Q¹ is iodo, Q², Q⁴, Q⁵ and Q⁶ are hydrogen and Q³ is chloro.

48. Compounds according to claim 45, wherein Q³ is hydrogen.

49. Compounds according to claim 1, wherein Q¹ is hydrogen, halogen, optionally substituted C₂₋₄ alkenyl, optionally substituted C₂₋₄ alkynyl, optionally substituted aryl or optionally substituted heteroaryl.

50. Compounds according to claim 1, wherein R¹ is C₁₋₄ alkyl.

51. Compounds according to claim 50, wherein R¹ is methyl or ethyl.

52. Compounds according to claim 51, wherein R¹ is methyl.

53. Compounds according to claim 51, wherein R¹ is ethyl.

54. Compounds according to claim 1, wherein R¹ is methyl or ethyl, Q¹ is hydrogen or halogen, Q² is hydrogen, C₁₋₄ alkyl or halogen and Q³ is hydrogen or halogen.

55. Compounds according to claim 54, wherein Q¹ is chloro, bromo or iodo, Q² is hydrogen, methyl, ethyl, chloro or bromo, and Q³ is fluoro or bromo.

56. Compounds according to claim 1, wherein R² is hydrogen or methyl.

57. Compounds according to claim 56, wherein R² is hydrogen.

58. Compounds according to claim 1, wherein R¹ is methyl or ethyl, R² is hydrogen, Q¹ is hydrogen or halogen, Q² is hydrogen, C₁₋₄ alkyl or halogen and Q³ is hydrogen or halogen.

59. Compounds according to claim 58, wherein Q¹ is chloro, bromo or iodo, Q² is hydrogen, methyl, ethyl, chloro or bromo, and Q³ is hydrogen, fluoro or bromo.

60. Compounds according to claim 1, wherein R³ is tert-butyl, 1-halo-2-methylprop-2-yl, 1,1-dihalo-2-methylprop-2-yl, 1,1,1-trihalo-2-methylprop-2-yl, 1-alkoxy-2-methylprop-2-yl, 1-alkenyloxy-2-methylprop-2-yl, 1-alkynyloxy-2-methylprop-2-yl, 1-cyano-2-methyl-prop-2-yl, 1-alkoxyalkoxy-2-methyl-prop-2-yl, 1-halo-3-methylbut-3-yl, 1-alkoxy-3-methylbut-3-yl, 1-alkenyloxy-3-methylbut-3-yl, 1-alkynyloxy-3-methylbut-3-yl, 1-cyano-3-methylbut-3-yl, 2-cyanoprop-2-yl, 2-methoxycarbonylprop-2-yl, 2-(C₁₋₂)alkoxycarbonylprop-2-yl or 2-methylaminocarbonylprop-2-yl, 1-alkylthio-2-methylprop-2-yl, 2-cyano-1-alkoxyprop-2-yl, 2-cyano-1-haloprop-2-yl, 1-alkoxy-prop-2-yl, 1-halo-prop-2-yl, 1-cyanoalkyl-3-methylbut-3-yl, 1-haloalkyl-3-methylbut-3-yl, and R¹, R², Q¹, Q² and Q³ are as defined in claim 1.

61. Compounds according to claim 60, wherein R¹ is methyl or ethyl, R² is hydrogen, Q¹ is hydrogen or halogen, Q² is hydrogen, C₁₋₄ alkyl or halogen and Q³ is hydrogen or halogen.

62. Compounds according to claim 61, wherein Q¹ is chloro, bromo or iodo, Q² is hydrogen, methyl, ethyl, chloro or bromo, and Q³ is hydrogen, fluoro, chloro or bromo.

63. Compounds according to claim 60, wherein R³ is tert-butyl, 1-halo-2-methylprop-2-yl, 1-methoxy-2-methylprop-2-yl, 1-ethoxy-2-methylprop-2-yl, 1-allyloxy-2-methylprop-2-yl, 1-(prop-2-ynyloxy)-2-methylprop-2-yl, 2-cyano-1-methoxyprop-2-yl, 2-cyano-1-haloprop-2-yl, 2-cyano-1-ethoxyprop-2-yl, 2-cyano-1-(prop-2-ynyloxy)-prop-2-yl, and R¹, R², Q¹, Q² and Q³ are as defined in claim 1.

64. Compounds according to claims 1, wherein R⁴ is C₁₋₆ alkyl optionally substituted with C₁₋₄alkoxy-(C₁₋₄)alkoxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with benzyloxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with C₂₋₆ alkenyloxy, C₂₋₆ alkynyloxy or —S(O)ₓ(C₁₋₆)alkyl, wherein x is 0, 1 or 2 and the alkyl group is optionally substituted with halo, mono- or di-(C₁₋₆)alkylamino, R⁴ is —CH₂—C≡C—R⁵, wherein R⁵ is hydrogen, C₁₋₈ alkyl optionally substituted with halogen, hydroxy, C₁₋₆ alkoxy, C₁₋₃alkoxy(C₁₋₃)alkoxy, cyano and R¹, R², Q¹, Q² and Q³ are as defined in claim 1.

65. Compounds according to claim 64, wherein R¹ is methyl or ethyl, R² is hydrogen, Q¹ is hydrogen or halogen, Q² is hydrogen, C₁₋₄ alkyl or halogen and Q³ is hydrogen or halogen.

66. Compounds according to claim 65, wherein Q¹ is chloro, bromo or iodo, Q² is hydrogen, methyl, ethyl, chloro or bromo, and Q³ is hydrogen, fluoro, chloro or bromo.

67. Compounds according to claim 1, wherein the optionally substituted aryl and optionally substituted heteroaryl rings or moieties of the R₅ values are optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)alkenyloxy, C₂₋₆ alkynyloxy, halo(C₂₋₆)alkynyloxy, aryl, aryloxy, aryl(C₁₋₆)alkyl, aryl(C₁₋₆)alkoxy, heteroaryl, heteroaryloxy, heteroaryl(C₁₋₆)alkyl, heteroaryl(C₁₋₆)alkoxy, —SF₅, —S(O)_{g}(C₁₋₄)alkyl wherein g is 0, 1 or 2 and the alkyl is optionally substituted with halo, or R⁵ is optionally substituted with —OSO₂(C₁₋₄)alkyl, wherein the alkyl group is optionally substituted with halo, or R⁵ is optionally substituted with —CONR^{g}R^{h}, —COR^{g}, —CO₂R^{g}, —R^{gg}═NR^{h}, —NR^{g}R^{h}, —NR^{g}COR^{h}, —NR^{g}CO₂R^{h}, —SO₂NR^{g}R^{h} or —NR^{g}SO₂Rⁱ, wherein Rⁱ is C₁₋₆ alkyl optionally substituted with halogen, R^{gg} is (C₁₋₆)alkylene, and R^{g} and R^{h}, independently of each other, are hydrogen or C₁₋₆ alkyl optionally substituted with halogen, or, in the case of —CONR^{g}R^{h} or —SO₂NR^{g}R^{h}, R^{g}R^{h} may join to form a 5- or 6-membered carbocyclic or heterocyclic ring containing a heteroatom selected from sulphur, oxygen or NR⁰, wherein R⁰ is hydrogen or optionally substituted C₁₋₆alkyl and R¹, R², Q¹, Q² and Q³ are as defined in claim 1.

68. Compounds according to claim 67, wherein R¹ is methyl or ethyl, R² is hydrogen, Q¹ is hydrogen or halogen, Q² is hydrogen, C₁₋₄ alkyl or halogen and Q³ is hydrogen or halogen.

69. Compounds according to claim 67, wherein Q¹ is chloro, bromo or iodo, Q² is hydrogen, methyl, ethyl, chloro or bromo, and Q³ is hydrogen, fluoro, chloro or bromo.

70. Compounds according to claim 1, wherein the optionally substituted aryl, optionally substituted heteroaryl or optionally substituted 5- to 8-membered ring R⁴ is optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)alkenyloxy, C₂₋₆ alkynyloxy, halo(C₂₋₆)alkynyloxy, —SF₅, —S(O)ₓ(C₁₋₆)alkyl, wherein x is 0, 1 or 2 and the alkyl group is optionally substituted with halo, or R⁴ is optionally substituted with —OSO₂(C₁₋₄)alkyl, wherein the alkyl group is optionally substituted with halogen, —CONR^{x}R^{y}, —CON(OR^{x})R^{y}, —COR^{x}, —CO₂R^{x}, —CR^{x}═NR^{y}, —NR^{x}R^{y}, —NR^{x}COR^{y}, —NR^{x}CO₂R^{y}, —SO₂NR^{x}R^{y} or —NR^{x}SO₂R^{z}, wherein R^{z} is C₁₋₈ alkyl optionally substituted with halogen and R^{x} and R^{y}, independently of each other, are hydrogen or C₁₋₆ alkyl optionally substituted with halogen and R¹, R², Q¹, Q² and Q³ are as defined in claim 1.

71. Compounds according to claim 70, wherein R¹ is methyl or ethyl, R² is hydrogen, Q¹ is hydrogen or halogen, Q² is hydrogen, C₁₋₄ alkyl or halogen and Q³ is hydrogen or halogen.

72. Compounds according to claim 71, wherein Q¹ is chloro, bromo or iodo, Q² is hydrogen, methyl, ethyl, chloro or bromo, and Q³ is hydrogen, fluoro, chloro or bromo.

73. Compounds according to claim 1, wherein L is oxygen.

74. Compounds according to claim 1, wherein m is 0.

75. Compounds according to claim 1, wherein m is 1.

76. A process for preparing a compound according to claim 1 as described herein.

77. A fungicidal composition comprising a fungicidally effective amount of a compound according to claim 1 and a suitable carrier or diluent therefor.

78. A method of combating or controlling phytopathogenic fungi which comprises applying a fungicidally effective amount of a compound according to claim 1 to a plant, to a seed of a plant, to the locus of the plant or seed or to soil or any other plant growth medium.
